(19) United States
(12) Patent Application Publication
(10) Pub. No.: US 2010/0291575 A1
(43) Pub. Date: Nov. 18, 2010

(54) Detection of Changes in Cell Populations and Mixed Cell Populations

(75) Inventors: Steven Shamah, Acton, MA (US); Lance G. Laing, Belmont, MA (US); Alexander Yuzhakov, West Roxbury, MA (US); Rick Wagner, Cambridge, MA (US); Marla Abodeely, Cambridge, MA (US); Bennett Rockney, Westford, MA (US); Stephen C. Schulz, Lee, NH (US); Zinkal Padalia, North Andover, MA (US); Michael Getman, Ashland, MA (US); Eric Sandberg, DuPont, WA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: SRU Biosystems, Inc.

(21) Appl. No.: 12/781,513

(22) Filed: May 17, 2010

Related U.S. Application Data
(60) Provisional application No. 61/178,787, filed on May 15, 2009.
(60) Provisional application No. 61/257,345, filed on Nov. 2, 2009.
(60) Provisional application No. 61/296,099, filed on Jan. 19, 2010.
(60) Provisional application No. 61/323,070, filed on Apr. 12, 2010.
(60) Provisional application No. 61/315,144, filed on Mar. 18, 2010.

(51) Int. Cl. C12Q 1/68; G01N 33/567; C12Q 1/02

(52) U.S. Cl. 435/6; 435/7.2; 435/7.21; 435/29

(57) ABSTRACT

The invention provides methods of label-free detection of changes in cell populations and mixed cell populations.

### PRIORITY

This application claims the benefit of the following provisional applications: U.S. Ser. No. 61/178,787, filed May 15, 2009, U.S. Ser. No. 61/257,345, filed Nov. 2, 2009, U.S. Ser. No. 61/296,099, filed Jan. 19, 2010, U.S. Ser. No. 61/315,144, filed Mar. 18, 2010, and U.S. Ser. No. 61/323,070, filed Apr. 12, 2010, all of which are incorporated by reference in their entirety.

### BACKGROUND OF THE INVENTION

Cell analysis, in particular, stem cell analysis, primary cell analysis, and mixed cell population analysis, is currently limited in the field due to the lack of tools available to accurately measure real time biological processes, such as adhesion, cell migration and chemotaxis, invasion into basement membranes or tissues, differentiation, differentiation mediated by cellular adhesion, differentiation mediated by tertiary environments (3-D cell culture), and differentiation mediated by co-culture with different cell types, in particular when cell numbers are scarce.

Disclosed herein are methods that solve each of these problems using label-free detection in real time using live cells, including stem cells, primary cells, and mixed populations of cells.

Additionally, preparation of biological samples for analysis can be time consuming and complicated. The separation and manipulation of living cells is an initial step for many biological and medical analyses, including isolation and detection of cancer cells, concentration of cells from dilute suspensions, separation of cells according to specific properties, and isolation and positioning of individual cells for analyses.

Flow cytometry and fluorescence-activated cell sorters (FACS) are widely used for cell sorting and cell analyses. However, these methods are expensive, require detectable labels, can damage the cells leaving them unusable for further analysis, and require relatively large sample volumes. Furthermore, the devices are difficult to sterilize, mechanically complicated, and can only be operated and maintained by trained personnel. Therefore, inexpensive devices that can rapidly and efficiently sort, enumerate, detect and analyze live cells, including mixed populations of live cells and low cell number assays, are needed for biological science research and medical diagnosis.

### SUMMARY OF THE INVENTION

One embodiment of the invention provides a method for detecting differential responses of two or more types of cells in one vessel to stimuli or a test reagent, wherein the two or more types of cells do not comprise detectable labels. The method comprises applying the two or more types of cells to the one vessel, wherein the vessel comprises a colorimetric resonant reflectance biosensor surface, a grating-based waveguide biosensor surface, or a dielectric film stack biosensor surface, wherein the biosensor surface has one or more specific binding substances immobilized to its surface and wherein the one or more specific binding substances can bind one or more of the two or more types of cells. The two or more types of cells are allowed to bind to the one or more specific binding substances. The differential responses of the two or more cell types are detected. The differential responses can be different times of the two or more types of cells to attach to the one or more specific binding substances; different cell attachment morphologies displayed by the two or more types of cells to the one or more specific binding substances; and/or different strengths of attachment of the two or more cell types to the one or more specific binding substances.

The method can further comprise exposing the two or more cell types to one or more test reagents or stimuli and detecting the differential responses of the two or more cell types to the one or more test reagents or stimuli. The differential responses can be different strengths of response of the two or more cell types to the one or more test reagents or stimuli; different cell morphologies displayed by the two or more types of cells in response to one or more test reagents or stimuli; different cell responses of the two or more cell types to the one or more test reagents or stimuli over time; and/or different response kinetics of the two or more cell types over time.

The method can further comprise exposing the two or more cell types to a first test reagent or first stimuli; detecting the responses of the two or more cell types to the first test reagent or first stimuli; exposing the two or more cell types to a second test reagent or second stimuli, wherein the response of one of the cell types in the two or more cell types to the second test reagent or second stimuli is known; detecting the responses of the two or more cell types to the second test reagent or second stimuli; identifying on the biosensor the one of the cell types in the two or more cell types that have a known response to the second test reagent or second stimuli; and detecting the differential response of the two or more types of cells. The one or more test reagents or stimuli can be expressed by one or more cells of the two or more types of cells present on the biosensor surface.

Another embodiment of the invention comprises a method of detecting the presence of a first cell type in a mixed population of cells, wherein the cells in the mixed population of cells do not comprise detectable labels. The method comprises applying the mixed population of cells to one vessel, wherein the vessel comprises a colorimetric resonant reflectance biosensor surface, a grating-based waveguide biosensor surface, or a dielectric film stack biosensor surface, wherein the biosensor surface has one or more specific binding substances immobilized to its surface. The mixed population of cells is allowed to bind to the one or more specific binding substances, wherein the first cell type has a differential response from the other cells of the mixed population of cells to binding to the one or more specific binding substances. Differential responses of the mixed population of cells are detected, wherein the presence of the first type of cells is detected by their differential response. The differential response can be a different time of the first cell type to attach to the one or more specific binding substances; a different cell attachment morphology displayed by the first type of cells to the one or more specific binding substances; a different strength of attachment of the first type of cells to the one or more specific binding substance; and/or a different response of the first type of cells over time. The percentage of the first type of cells in the mixed population of cells can be determined.

Yet another embodiment of the invention provides a method of detecting the presence of a first cell type in a mixed population of cells, wherein none of the cells in the mixed population of cells comprise detectable labels. The method comprises applying the mixed population of cells to one vessel, wherein the vessel comprises a colorimetric resonant reflectance biosensor surface, a grating-based waveguide biosensor surface, or a dielectric film stack biosensor surface, wherein the biosensor surface has one or more specific binding substances immobilized to its surface. The mixed population of cells is allowed to bind to the one or more specific binding substances. The mixed population of cells to is exposed to one or more test regents or stimuli, wherein the first cell type has a differential response to the one or more test reagents or stimuli as compared to the other cells in the mixed population of cells. The differential response of the first cell type to the one or more test reagents or stimuli is detected, wherein if the differential response is detected, then the first cell type is present in the mixture of cells. The differential response is a different strength of response of the first cell type to the one or more test reagents or stimuli; a different cell morphology displayed by the first cell type in response to one or more test reagents or stimuli; a different cell response of the first cell type to the one or more test reagents or stimuli over time; and/or a different response kinetic of the first type of cells over time. The percentage of the first type of cells in the mixed population of cells can be determined. The one or more test reagents or stimuli can be expressed by one or more cells of the mixed population of cells present on the biosensor surface.

Still another embodiment of the invention provides a method of detection of responses of a first population of cells to one or more test reagents or stimuli. The method comprises immobilizing one or more extracellular matrix ligands to a surface of a colorimetric resonant reflectance biosensor, a grating-based waveguide biosensor, or a dielectric film stack biosensor, wherein the first population of cells have cell surface receptors specific for the one or more extracellular matrix ligands; and adding the first population of cells to the biosensor. Alternatively, the first population of cells can be mixed with one or more extracellular matrix ligands, wherein the first population of cells has cell surface receptors specific for the one or more extracellular matrix ligands; and added to a surface of the colorimetric resonant reflectance biosensor, the grating-based waveguide biosensor, or the dielectric film stack biosensor. A gel, gel-like substance, or a second population of cells is added to the biosensor surface. The one or more test reagents or stimuli are added to the gel or gel-like substance, or the second population of cells. Responses of the first population of cells to the one or more test reagents or stimuli are detected. The one or more test reagents or stimuli can be a chemotactic agent or a third population of cells that produce test reagents or stimuli. The second population of cells can be a population of epithelial cells or a population of endothelial cells. The first population of cells can be a population of stem cells. No detection labels can be used. The method can further comprising detecting the responses of the second population of cells. The responses of the first population of cells or second population of cells to the one or more stimuli can be detected by monitoring the peak wavelength value over one or more time periods or by monitoring the change in effective refractive index over one or more time periods. The responses of the first population of cells or second population of cells can be detected in real time.

Even another embodiment of the invention provides a method of detection of responses of a first population cells to a one or more test reagents or stimuli. The method comprises adding one or more test reagents or stimuli to a surface of a colorimetric resonant reflectance biosensor, a grating-based waveguide biosensor, or dielectric film stack biosensor; adding basement membrane matrix, alginate gel, collagen gel, agarose gel, synthetic hydrogel, or a second population of cells to the biosensor surface; mixing the first population of cells with one or more extracellular matrix ligands, wherein the first population of cells have cell surface receptors specific for the one or more extracellular matrix ligands; and adding the first population of cells to the biosensor; detecting the responses of the first population cells to the one or more test reagents or stimuli. The one or more test reagents or stimuli can be a chemotactic agent or a third population of cells that produce test reagents or stimuli. The second population of cells can be a population of epithelial cells or a population of endothelial cells. The first population of cells can be a population of stem cells. No detection labels can be used. The method can further comprise detecting the responses of the second population of cells. The responses of the first population of cells or second population of cells to the one or more stimuli can be detected by monitoring the peak wavelength value over one or more time periods or by monitoring the change in effective refractive index over one or more time periods. The responses of the first population of cells or second population of cells can be detected in real time.

Another embodiment of the invention provides a method of detection of differentiation of a first population of cells. The method comprises adding the first population of cells to a surface of a colorimetric resonant reflectance biosensor or a dielectric film stack biosensor, wherein the biosensor has two or more surface sectors, wherein each surface sector has a grating that with a different resonance value than the other surface sectors; detecting two of more peak wavelength values from each of the two or more surface sectors; and detecting differentiation of the first population of cells on the biosensor surface. The differentiation can be detected in real time. The one or more test reagents or stimuli can be applied to the biosensor before the detection of two or more peak wavelength values from each of the two or more surface sectors. The one or more peak wavelength values can be detected before the one or more test reagents or stimuli are applied to the biosensor. The one or more test reagents or stimuli can be a chemotactic agent or a third population of cells that produce test reagents or stimuli. The first population of cells can be a population of stem cells. No detection labels can be used.

Even another embodiment of the invention provides a method of biological expression profiling to identify biological response signatures specific for a particular population of stem cells. The method comprises immobilizing one or more extracellular matrix ligands to two or more surfaces of a colorimetric resonant reflectance biosensor, a grating-based waveguide biosensor, or a dielectric film stack biosensor, wherein the population of stem cells have cell surface receptors specific for the one or more extracellular matrix ligands; and adding the population of stem cells to the two or more locations of the biosensor. Alternatively, the population of stem cells can be mixed with one or more extracellular matrix ligands, wherein the stem cells have cell surface receptors specific for the one or more extracellular matrix ligands; and added to two or more surfaces of a colorimetric resonant reflectance biosensor, a grating-based waveguide biosensor or a dielectric film stack biosensor. The two or more surfaces of the biosensor are exposed to two or more test reagents or stimuli. The responses of the stem cells to the test reagents or stimuli are detected at each of the two or more surfaces of the biosensor. The biological response signatures specific for a particular population of the stem cells to two or more test reagents or stimuli are identified. Detecting responses of the stem cells can be done in real time.

Still another embodiment of the invention provides a method for screening a candidate compound for its ability to modulate cell differentiation. The method comprises adding one or more types of cells to a surface of a colorimetric resonant reflectance biosensor, a grating-based waveguide biosensor, or a dielectric film stack biosensor; inducing the one or more types of cells to differentiate; and detecting a change in cell differentiation in the presence or absence of the candidate compound by comparing the peak wavelength values or effective changes in refractive index in the presence or absence of the candidate compound. A change in cell differentiation activity in the presence of the compound relative to cell differentiation activity in the absence of the candidate compound indicates an ability of the candidate compound to modulate cell differentiation. The change in cell differentiation activity can be an increase in cell differentiation activity, decrease in cell differentiation activity, inhibition of cell differentiation activity, increase or decrease in stem cell self-renewal, and/or a change in the type of differentiated cell. The change in cell differentiation activity can be an increase or decrease in collagen production. The change in cell differentiation activity can be an increase or decrease in mineralized nodule formation. The one or more types of cells can be stem cells. The one or more types of cells can be mesenchymal stem cells. The change in cell differention activity can be detected by detecting a change in cell size, cell shape, cell membrane potential, cell metabolic activity, or cell responsiveness to signals. The candidate compound can be an inhibitory nucleic acid molecule.

Yet another embodiment of the invention provides a method for screening a candidate compound for its ability to modulate cell differentiation. The method comprises adding one or more types of cells to a surface of a colorimetric resonant reflectance biosensor, a grating-based waveguide biosensor, or a or a dielectric film stack biosensor; inducing the one or more types of cells to differentiate; and detecting the production of one or more cell products of differentiation in the presence or absence of the candidate compound by comparing the peak wavelength values or effective refractive index in the presence or absence of the candidate compound. A change in one or more cell products of differentiation in the presence of the candidate compound relative to one or more cell products of differentiation in the absence of the candidate compound indicates an ability of the candidate to modulate cell differentiation. The product of cell differentiation can be collagen or mineralization nodules. The one or more types of cells can be stem cells. The one or more types of cells can be mesenchymal stem cells. The candidate compound can be an inhibitory nucleic acid molecule.

Another embodiment of the invention provides a colorimetric resonant reflectance biosensor grating surface, a grating-based waveguide biosensor grating surface, or a or a dielectric film stack biosensor grating surface comprising: one or more specific binding substances immobilized to or associated with the biosensor grating surface; and a layer of a gel or gel-like substance over the one or more specific binding substances. The biosensor grating surface can form an internal surface of a liquid containing vessel. The liquid containing vessel can be a microtiter plate or a microfluidic channel.

Even another embodiment of the invention provides a kit comprising one or more colorimetric resonant reflectance biosensor grating surfaces, one or more grating-based waveguide biosensor grating surfaces, or a dielectric film stack biosensor grating surfaces and one or more containers of gel or gel-like substances. The kit can further comprise a container of one or more specific binding substances. The one or more colorimetric resonant reflectance biosensor grating surfaces, grating-based waveguide biosensor grating surfaces, or a dielectric film stack biosensor grating surfaces can comprise one or more specific binding substances immobilized to or associated with the biosensor grating surface.

Still another embodiment of the invention provides an improved method for detecting reactions between a specific binding substance and a binding partner on a colorimetric resonant reflectance biosensor grating surface, grating-based waveguide biosensor grating surface, or a dielectric film stack biosensor grating surface. The method comprises applying one or more specific binding substances to the biosensor grating surface such that the one or more specific binding substances become immobilized to or associated with the biosensor grating surface and applying a gel or gel like substance to the biosensor surface.

Yet another embodiment of the invention provides a method of sorting two or more cell types from a mixed population of cells and detecting the response of the sorted cells to stimuli, incubation, or a test reagent, wherein the sorting and the detection occur on one biosensor surface. The method comprises applying a mixed population of cells to one colorimetric resonant reflectance biosensor surface, one grating-based waveguide biosensor surface, or one dielectric film stack biosensor surface wherein the one biosensor surface has two or more types of specific binding substances immobilized to its one surface, and wherein the two or more specific binding substances can potentially bind one or more cell types in the mixed population of cells; washing the unbound cells from the one surface of the biosensor, such that one or more cell types are bound to and sorted on the surface of the biosensor; exposing the one or more bound cell types to stimuli, incubation, or a test reagent; and detecting the response of the one or more bound cell types to the stimuli, incubation, or the test reagent. The two or more specific binding substances can comprise a combination of one or more extracellular matrix proteins and one or more other specific binding substances. The one biosensor surface can be the bottom of a microtiter well. The two or more cell types and test reagent do not comprise detectable labels.

Another embodiment of the invention provides a method of sorting one or more cell types from a mixed population of cells and detecting an intracellular analyte from the one or more cell types on one biosensor surface. The method comprises applying a mixed population of cells to one colorimetric resonant reflectance biosensor surface, one grating-based waveguide biosensor surface, or one dielectric film stack biosensor surface wherein the one biosensor surface has two or more specific binding substances immobilized to its one surface, wherein the two or more specific binding substances comprise (i) first specific binding substances that specifically bind one or more cell types in the mixed population of cells and (ii) second specific binding substances that specifically bind one or more intracellular analytes from the one or more cell types; washing the unbound cells from the surface of the biosensor, such that the one or more cell types are bound to and sorted on the surface of the biosensor; lysing or permeabilizing the one or more bound cell types; washing any unbound analytes from the surface of the biosensor; and detecting the intracellular analytes immobilized to the surface of the biosensor. The first specific binding substances can comprise one or more extracellular matrix proteins. The cells can be incubated for a period of time, or exposed to stimuli, or exposed to a test reagent prior to lysing or permeabilizing of the one or more bound cell types. The one biosensor surface can be the bottom of a microtiter well. The mixed population of cells and the two or more specific binding substances do not comprise detectable labels.

Even another embodiment of the invention provides a method of sorting one or more cell types from a mixed population of cells and detecting an analyte from the one or more cell types on one biosensor surface. The method comprises applying a mixed population of cells to one colorimetric resonant reflectance biosensor surface, one grating-based waveguide biosensor surface, or one dielectric film stack biosensor surface, wherein the one biosensor surface has two or more specific binding substances immobilized to its one surface, wherein the two or more specific binding substances comprise (i) first specific binding substances that specifically bind one or more cell types in the mixed population of cells and (ii) second specific binding substances that specifically bind one or more analytes from the one or more cell types; washing the unbound cells from the surface of the biosensor, such that the one or more cell types are bound to and sorted on the surface of the biosensor; applying a test reagent to the cells, or incubating the cells, or subjecting the cells to stimuli or a combination thereof; and detecting the analytes immobilized to the surface of the biosensor. The first specific binding substances can be one or more extracellular matrix proteins. The one biosensor surface can be the bottom of a microtiter well. The mixed population of cells and the two or more specific binding substances do not comprise detectable labels.

### BRIEF DESCRIPTION OF THE DRAWINGS

FIG. 1 shows the signature response for SH-SY5Y cells to muscarinic, P2Y, and beta-arrestin ligands on a colorimetric resonant reflectance biosensor microwell plate.

FIG. 2 shows the reaction of mP-M5 and mP-M4 cells to 3 ligands: acetylcholine, carbachol, and pilocarpine when the cells are on colorimetric resonant reflectance biosensors comprising PBS/ovalbumin, fibronectin, collagen or laminin.

FIG. 3A shows the signal generated by M5 cells attaching to a colorimetric resonant reflectance biosensor. FIG. 3B shows a scan that was completed 30 minutes after the cells attached to the biosensor.

FIG. 4A shows a phase contrast image of cells from the top side of the cells (side opposite of the cell attachment to the colorimetric resonant reflectance biosensor), while the FIG. 4B shows the attachment signal of the same cells from the bottom side of the cells (the side of the cell that is bound to the biosensor).

FIG. 5A shows the attachment response of M5 cells to a colorimetric resonant reflectance biosensor. FIG. 5B shows the response of the M5 cells to the addition of carbachol.

FIG. 6 shows a mixed population of M4 cells and RBL parental cells that were added to a colorimetric resonant reflectance biosensor. M4 cells have more receptors for carbachol than the RBL cells. 10 μM of carbachol was then added to the cells. The middle panel shows a 3:1 ratio of M4 cells to RBL cells 30 minutes after the carbachol is added to the cells. The right panel shows a 1:3 ratio of M4 cells to RBL cells 30 minutes after the carbachol is added. The middle panel of FIG. 6 shows more signal than the right panel because more M4 cells are present than RBL cells, each M4 cell having more receptors for carbachol.

FIG. 7 shows the rat MSC cell attachment to colorimetric resonant reflectance biosensors comprising either ovalbumin, fibronectin, laminin or collagen.

FIG. 8 shows rat MSC cells shortly after adding the cells to the colorimetric resonant reflectance biosensor (FIG. 8A) and after 16 hours on the biosensor (FIG. 8B).

FIG. 9 shows movement of rat MSC cells over 30 hours on the colorimetric resonant reflectance biosensor surface. The arrow on the left (pointing to a dark spot) demonstrates where the cell was shortly after it attached to the biosensor surface and the arrow on the right (pointing to a light spot) demonstrates where the cell was 30 hours after attachment to the biosensor surface.

FIG. 10 shows the response of THP-1 cells (FIG. 10A) and OEM cells (FIG. 10B) to different concentrations of SDF-1a using a colorimetric resonant reflectance biosensor microwell plates and a BIND® READER.

FIG. 11A shows the response of MSC cells to SDF-1α on colorimetric resonant reflectance biosensor microwell plate. FIG. 11B shows the response of MSC cells (7,000 cells in a 384 well microplate) to SDF-1α and inhibitors (CXCR4 blocking antibodies).

FIG. 12 shows rat MSC cells on a biosensor coated with fibronectin. Cell attachment was detected on a colorimetric resonant reflectance biosensor at 3 hours and 16 hours (left panels). The attachment signal was zeroed out and the cells were stimulated with SDF-1α or were not stimulated (right panels). Movement of the cells can be seen in the right panels of FIG. 12. The darker spots are where the cells were prior to detection and the lighter spots are where the cells are when the reaction was detected. Where no stimulus was added to the cells, some movement of the cells can be seen; however, where SDF-1α was added to the cells movement of the cells is seen along with a spreading out of the cells on the biosensor.

FIG. 13A-B shows an enlargement of the right panels of FIG. 12. An enhanced signal can be seen on the cell edges where movement and/or cell adhesion is occurring. The enhanced signal correlates with the leading edge of the cells as they move across the biosensor as evidenced by time lapse imaging.

FIG. 14 demonstrates the reading from the BIND® READER (FIG. 14A) and the BIND® SCANNER (FIG. 14B). An approximately 7 to 10 fold improvement in signal to noise is observed.

FIG. 15 shows a schematic diagram of a lift-off assay.

FIG. 16 shows MSC cells lifting up off the biosensor in the presence of MATRIGEL™ basement membrane matrix as compared to control wells. The MSC attachment signal can be readily identified with the MATRIGEL™ coating. The MSCs display a tendency to lift up off the sensor as compared to control wells. This is evidenced by a negative PWV shift displayed as black in FIG. 16.

FIG. 17 shows rat MSCs that were induced to differentiate into osteoblasts on a biosensor coated with collagen. By day 14 the cells were mineralizing and producing bone.

FIG. 18 shows rat MSCs that were induced to differentiate into osteoblasts on a biosensor coated with collagen. By day 14 the cells were mineralizing and producing bone. Alizarin red dye was used to confirm that the cells were indeed producing bone. The images were baselined from the previous day.

FIG. 19A shows a close up of the day 17 panel from FIG. 18. The white area is mineralization of the osteoblasts. FIG. 19B shows a phase contrast micrograph of the same portion of cells. The phase contrast micrograph does not show the differentiation of the cells.

FIG. 20 shows rat MSCs (Invitrogen) seeded in 384-well colorimetric resonant reflectance biosensors at 100 cells/well and treated with osteoblast differentiation media. Daily images were acquired on the BIND® SCANNER and baselined to the Day 0 cell attachment signal. A gradual and robust PWV shift (˜25 nM) was detected as bone-like minerals are deposited on the sensor surface, as indicated by alizarin red staining of parallel wells (FIG. 20A). An inhibitor of glycogen synthase kinase 3 (GSK3R) expedites MSC-osteoblast differentiation. FIG. 20B demonstrates the detection of the expedited differentiation caused by GSK3R. FIG. 20C demonstrates that the BIND® SCANNER is more sensitive than alizarin red staining in detecting mineralization.

FIG. 21 shows differentiating MSCs on BIND™ biosensors. Collagen formation is shown to precede mineralization, which is consistent with normal bone formation.

FIG. 22 shows rat MSCs cultured in osteoblast differentiation media with or without GSK3β inhibitor for 1 to 19 days. BIND™ images were collected daily and baselined to previous day measurements, thus providing information on the rate of mineralization (FIG. 22A). FIG. 22B shows the quantitation of PWV shifts as measured on BIND® SCANNER (+/− standard deviation, n=12 wells).

FIG. 23 shows antibody blocking of MSC migration and also shows a very bright oblong of positive PWV shift in the center of the well representing the interaction of PDGF-BB antibody with the PDGF-BB spotted on the biosensor. In FIG. 23, “chemokine X” is PDGF-BB; “chemokine X nAb” is neutralizing antibody specific for PDGF-BB.

FIG. 24 shows human MSCs seeded on a 384-well colorimetric resonant reflectance biosensor plate. The cells were treated with an osteoblast differentiation cocktail. PWVs were measured daily. Representative wells from untreated cells (Ctrl) and osteoblast-differentiated (OS-Diff) cells are shown.

FIG. 25 shows detection of accelerated osteoblast differentiation in label-free assays on the BIND® SCANNER when siRNA molecules specific for GSK3β and ADK were transfected into human MSCs just prior to differentiation. Sample wells at day 12 for several treatment conditions are shown.

FIG. 26 quantifies the results shown in FIG. 25.

FIG. 27 shows RBL and M5/RBL cells mixed in a 1:1 ratio and plated in colorimetric resonant reflectance biosensor wells. The cells were allowed to attach to the biosensor and the attachment reaction was detected on a BIND® SCANNER. The results are shown in FIG. 27A and FIG. 27B. The reaction of the cells to the acetylcholine is shown in FIG. 27C and FIG. 27D.

### DETAILED DESCRIPTION OF THE INVENTION

As used herein, the singular forms “a,” “an”, and “the” include plural referents unless the context clearly dictates otherwise.

### Biosensors

Biosensors of the invention can be colorimetric resonant reflectance biosensors. See e.g., Cunningham et al., “Colorimetric resonant reflection as a direct biochemical assay technique,” Sensors and Actuators B, Volume 81, p. 316-328, Jan. 5, 2002; U.S. Pat. Publ. No. 2004/0091397; U.S. Pat. No. 7,094,595; U.S. Pat. No. 7,264,973. Colorimetric resonant biosensors are not surface plasmon resonant (SPR) biosensors. SPR biosensors have a thin metal layer, such as silver, gold, copper, aluminum, sodium, and indium. The metal must have conduction band electrons capable of resonating with light at a suitable wavelength. A SPR biosensor surface exposed to light must be pure metal. Oxides, sulfides and other films interfere with SPR. Colorimetric resonant biosensors do not have a metal layer, rather they have a dielectric coating of high refractive index material, such as zinc sulfide, titanium dioxide, tantalum oxide, and silicon nitride.

Biosensors of the invention can also be dielectric film stack biosensors (see e.g., U.S. Pat. No. 6,320,991), diffraction grating biosensors (see e.g., U.S. Pat. No. 5,955,378; 6,100,991) and diffraction anomaly biosensors (see e.g., U.S. Pat. No. 5,925,878; RE37,473). Dielectric film stack biosensors comprise a stack of dielectric layers formed on a substrate having a grooved surface or grating surface (see e.g., U.S. Pat. No. 6,320,991). The biosensor receives light and, for at least one angle of incidence, a portion of the light propagates within the dielectric layers. The parameters of a sample medium are determined by detecting shifts in optical anomalies, i.e., shifts in a resonance peak or notch. Shifts in optical anomalies can be detected as either a shift in a resonance angle or a shift in resonance wavelength.

Other biosensors that can be used with the methods of the invention include grating-based waveguide biosensors, which are described in, e.g., U.S. Pat. No. 5,738,825. A grating-based waveguide biosensor comprises a waveguiding film and a diffraction grating that incouples an incident light field into the waveguiding film to generate a diffracted light field. A change in the effective refractive index of the waveguiding film is detected. Devices where the wave must be transported a significant distance within the device, such as grating-based waveguide biosensors, lack the spatial resolution of colorimetric resonant reflection biosensors.

A colorimetric resonant reflectance biosensor allows biochemical interactions to be measured on the biosensor's surface without the use of fluorescent tags, colorimetric labels or any other type of detection tag or detection label. Dielectric film stack biosensors work very similarly to colorimetric resonant reflectance biosensors. A biosensor surface contains an optical structure that, when illuminated with collimated and/or white light, is designed to reflect or transmit only a narrow band of wavelengths (“a resonant grating effect”). For reflection the narrow wavelength band is described as a wavelength “peak.” For transmission the narrow wavelength band is described as a wavelength “dip.” The “peak wavelength value” (PWV) changes when materials, such as biological materials, are deposited or removed from the biosensor surface. Wavelength dips can also be detected. A readout instrument is used to illuminate distinct locations on a biosensor surface with collimated and/or white light, and to collect reflected light. The collected light is gathered into a wavelength spectrometer for determination of a PWV.

A biosensor can be incorporated into standard disposable laboratory items such as microtiter plates by bonding the structure (biosensor side up) into the bottom of a bottomless microtiter plate cartridge. Incorporation of a biosensor into common laboratory format cartridges is desirable for compatibility with existing microtiter plate handling equipment such as mixers, incubators, and liquid dispensing equipment. Biosensors can also be incorporated into, e.g., microfluidic, macrofluidic, or microarray devices (see, e.g., U.S. Pat. No. 7,033,819, U.S. Pat. No. 7,033,821). Biosensors can be used with well-know methodology in the art (see, e.g., Methods of Molecular Biology edited by Jun-Lin Guan, Vol. 294, Humana Press, Totowa, N.J.) to monitor cell behavioral changes or the lack of these changes upon exposure to one or more extracellular reagents.

Colorimetric resonant reflectance biosensors comprise subwavelength structured surfaces (SWS) and are an unconventional type of diffractive optic that can mimic the effect of thin-film coatings. (Peng & Morris, “Resonant scattering from two-dimensional gratings,” J. Opt. Soc. Am. A, Vol. 13, No. 5, p. 993, May 1996; Magnusson, & Wang, “New principle for optical filters,” Appl. Phys. Lett., 61, No. 9, p. 1022, August, 1992; Peng & Morris, “Experimental demonstration of resonant anomalies in diffraction from two-dimensional gratings,” Optics Letters, Vol. 21, No. 8, p. 549, April, 1996). A SWS structure contains a one-dimensional, two-dimensional, or three dimensional grating in which the grating period is small compared to the wavelength of incident light so that no diffractive orders other than the reflected and transmitted zeroth orders are allowed to propagate. Propagation of guided modes in the lateral direction is not supported. Rather, the guided mode resonant effect occurs over a highly localized region of approximately 3 microns from the point that any photon enters the biosensor structure.

The reflected or transmitted light of a colorimetric resonant reflectance biosensor can be modulated by the addition of molecules such as ligands, specific binding substances, cells, or binding partners or both to the upper surface of the biosensor. The added molecules increase the optical path length of incident radiation through the structure, and thus modify the wavelength at which maximum reflectance or transmittance will occur.

In one embodiment, a colorimetric resonant reflectance biosensor, when illuminated with white and/or collimated light, is designed to reflect a single wavelength or a narrow band (e.g., about 1-10 nm) of wavelengths (a “resonant grating effect”). When mass is deposited on the surface of the biosensor, the reflected wavelength is shifted due to the change of the optical path of light that is shown on the biosensor.

A detection system consists of, for example, a light source that illuminates a small spot of a biosensor at normal incidence through, for example, a fiber optic probe, and a spectrometer that collects the reflected light through, for example, a second fiber optic probe also at normal incidence. Because no physical contact occurs between the excitation/detection system and the biosensor surface, no special coupling prisms are required and the biosensor can be easily adapted to any commonly used assay platform including, for example, microtiter plates. A single spectrometer reading can be performed in several milliseconds, thus it is possible to quickly measure a large number of molecular interactions taking place in parallel upon a biosensor surface, and to monitor reaction kinetics in real time.

A colorimetric resonant reflectance biosensor comprises, e.g., an optical grating comprised of a high refractive index material, a substrate layer that supports the grating, and optionally one or more specific binding substances or linkers immobilized on the surface of the grating opposite of the substrate layer. The high refractive index material has a higher refractive index than a substrate layer. See, e.g., U.S. Pat. No. 7,094,595; U.S. Pat. No. 7,070,987. Optionally, a cover layer covers the grating surface. An optical grating is coated with a high refractive index dielectric film which can be comprised of a material that includes, for example, zinc sulfide, titanium dioxide, tantalum oxide, silicon nitride, and silicon dioxide. A cross-sectional profile of a grating with optical features can comprise any periodically repeating function, for example, a “square-wave.” An optical grating can also comprise a repeating pattern of shapes selected from the group consisting of lines (one-dimensional), squares, circles, ellipses, triangles, trapezoids, sinusoidal waves, ovals, rectangles, and hexagons. A colorimetric resonant reflectance biosensor of the invention can also comprise an optical grating comprised of, for example, plastic or epoxy, which is coated with a high refractive index material.

Linear gratings (i.e., one dimensional gratings) have resonant characteristics where the illuminating light polarization is oriented perpendicular to the grating period. A colorimetric resonant reflection biosensor can also comprise, for example, a two-dimensional grating, e.g., a hexagonal array of holes or squares. Other shapes can be used as well. A linear grating has the same pitch (i.e. distance between regions of high and low refractive index), period, layer thicknesses, and material properties as a hexagonal array grating. However, light must be polarized perpendicular to the grating lines in order to be resonantly coupled into the optical structure. Therefore, a polarizing filter oriented with its polarization axis perpendicular to the linear grating must be inserted between the illumination source and the biosensor surface. Because only a small portion of the illuminating light source is correctly polarized, a longer integration time is required to collect an equivalent amount of resonantly reflected light compared to a hexagonal grating.

An optical grating can also comprise, for example, a “stepped” profile, in which high refractive index regions of a single, fixed height are embedded within a lower refractive index cover layer. The alternating regions of high and low refractive index provide an optical waveguide parallel to the top surface of the biosensor.

A colorimetric resonant reflectance biosensor of the invention can further comprise a cover layer on the surface of an optical grating opposite of a substrate layer. Where a cover layer is present, the one or more specific binding substances are immobilized on the surface of the cover layer opposite of the grating. Preferably, a cover layer comprises a material that has a lower refractive index than a material that comprises the grating. A cover layer can be comprised of, for example, glass (including spin-on glass (SOG)), epoxy, or plastic.

For example, various polymers that meet the refractive index requirement of a biosensor can be used for a cover layer. SOG can be used due to its favorable refractive index, ease of handling, and readiness of being activated with specific binding substances using the wealth of glass surface activation techniques. When the flatness of the biosensor surface is not an issue for a particular system setup, a grating structure of SiN/glass can directly be used as the sensing surface, the activation of which can be done using the same means as on a glass surface.

Resonant reflection can also be obtained without a planarizing cover layer over an optical grating. For example, a biosensor can contain only a substrate coated with a structured thin film layer of high refractive index material. Without the use of a planarizing cover layer, the surrounding medium (such as air or water) fills the grating. Therefore, specific binding substances are immobilized to the biosensor on all surfaces of an optical grating exposed to the specific binding substances, rather than only on an upper surface.

In general, a colorimetric resonant reflectance biosensor can be illuminated with white and/or collimated light that will contain light of every polarization angle. The orientation of the polarization angle with respect to repeating features in a biosensor grating will determine the resonance wavelength. For example, a “linear grating” (i.e., a one-dimensional grating) biosensor consisting of a set of repeating lines and spaces will have two optical polarizations that can generate separate resonant reflections. Light that is polarized perpendicularly to the lines is called “s-polarized,” while light that is polarized parallel to the lines is called “p-polarized.” Both the s and p components of incident light exist simultaneously in an unfiltered illumination beam, and each generates a separate resonant signal. A biosensor can generally be designed to optimize the properties of only one polarization (the s-polarization), and the non-optimized polarization is easily removed by a polarizing filter.

In order to remove the polarization dependence, so that every polarization angle generates the same resonant reflection spectra, an alternate biosensor structure can be used that consists of a set of concentric rings. In this structure, the difference between the inside diameter and the outside diameter of each concentric ring is equal to about one-half of a grating period. Each successive ring has an inside diameter that is about one grating period greater than the inside diameter of the previous ring. The concentric ring pattern extends to cover a single sensor location—such as an array spot or a microtiter plate well. Each separate microarray spot or microtiter plate well has a separate concentric ring pattern centered within it. All polarization directions of such a structure have the same cross-sectional profile. The concentric ring structure must be illuminated precisely on-center to preserve polarization independence. The grating period of a concentric ring structure is less than the wavelength of the resonantly reflected light. The grating period is about 0.01 micron to about 1 micron. The grating depth is about 0.01 to about 1 micron.

In another embodiment, an array of holes or posts are arranged to closely approximate the concentric circle structure described above without requiring the illumination beam to be centered upon any particular location of the grid. Such an array pattern is automatically generated by the optical interference of three laser beams incident on a surface from three directions at equal angles. In this pattern, the holes (or posts) are centered upon the corners of an array of closely packed hexagons. The holes or posts also occur in the center of each hexagon. Such a hexagonal grid of holes or posts has three polarization directions that “see” the same cross-sectional profile. The hexagonal grid structure, therefore, provides equivalent resonant reflection spectra using light of any polarization angle. Thus, no polarizing filter is required to remove unwanted reflected signal components. The period of the holes or posts can be about 0.01 microns to about 1 micron and the depth or height can be about 0.01 microns to about 1 micron.

A detection system can comprise a colorimetric resonant reflectance biosensor a light source that directs light to the colorimetric resonant reflectance biosensor, and a detector that detects light reflected from the biosensor. In one embodiment, it is possible to simplify the readout instrumentation by the application of a filter so that only positive results over a determined threshold trigger a detection.

By measuring the shift in resonant wavelength at each distinct location of a colorimetric resonant reflectance biosensor of the invention, it is possible to determine which distinct locations have, e.g., biological material deposited on them. The extent of the shift can be used to determine, e.g., the amount of binding partners in a test sample and the chemical affinity between one or more specific binding substances and the binding partners of the test sample.

A colorimetric resonant reflectance biosensor can be illuminated twice. The first measurement determines the reflectance spectra of one or more distinct locations of a biosensor with, e.g., before cells are added to the biosensor. The second measurement determines the reflectance spectra after, e.g., one or more cells are applied to a biosensor. The difference in peak wavelength between these two measurements is a measurement of the presence, amount, or status of cells on the biosensor. This method of illumination can control for small imperfections in a surface of a biosensor that can result in regions with slight variations in the peak resonant wavelength. This method can also control for varying concentrations or density of cell matter on a biosensor. A colorimetric resonant reflectance biosensor can also be illuminated greater than two times and the PWV determined and recorded. For example, the biosensor can be illuminated 1, 2, 4, 5, or 10 times a second, or 1, 2, 3, 4, 5, 10, 20, or 30 times a minute, or every 1, 5, 10, 20 or 60 minutes, or 1, 2, 3, 4, 5, 10 or more times a day.

### Detection Systems

A detection system can comprise a biosensor a light source that directs light to the biosensor, and a detector that detects light reflected from the biosensor. In one embodiment, it is possible to simplify the readout instrumentation by the application of a filter so that only positive results over a determined threshold trigger a detection.

A light source can illuminate a colorimetric resonant reflectance biosensor from its top surface, i.e., the surface to which one or more specific binding substances are immobilized or from its bottom surface. By measuring the shift in resonant wavelength at each distinct location of a biosensor of the invention, it is possible to determine which distinct locations have binding partners bound to them. The extent of the shift can be used to determine the amount of binding partners in a test sample and the chemical affinity between one or more specific binding substances and the binding partners of the test sample.

One type of detection system for illuminating the biosensor surface and for collecting the reflected light is a probe containing, for example, six illuminating optical fibers that are connected to a light source, and a single collecting optical fiber connected to a spectrometer. The number of fibers is not critical, any number of illuminating or collecting fibers are possible. The fibers are arranged in a bundle so that the collecting fiber is in the center of the bundle, and is surrounded by the six illuminating fibers. The tip of the fiber bundle is connected to a collimating lens that focuses the illumination onto the surface of the biosensor.

In this probe arrangement, the illuminating and collecting fibers are side-by-side. Therefore, when the collimating lens is correctly adjusted to focus light onto the biosensor surface, one observes six clearly defined circular regions of illumination, and a central dark region. Because the biosensor does not scatter light, but rather reflects a collimated beam, no light is incident upon the collecting fiber, and no resonant signal is observed. Only by defocusing the collimating lens until the six illumination regions overlap into the central region is any light reflected into the collecting fiber. Because only defocused, slightly uncollimated light can produce a signal, the biosensor is not illuminated with a single angle of incidence, but with a range of incident angles. The range of incident angles results in a mixture of resonant wavelengths. Thus, wider resonant peaks are measured than might otherwise be possible.

Therefore, it is desirable for the illuminating and collecting fiber probes to spatially share the same optical path. Several methods can be used to co-locate the illuminating and collecting optical paths. For example, a single illuminating fiber, which is connected at its first end to a light source that directs light at the biosensor, and a single collecting fiber, which is connected at its first end to a detector that detects light reflected from the biosensor, can each be connected at their second ends to a third fiber probe that can act as both an illuminator and a collector. The third fiber probe is oriented at a normal angle of incidence to the biosensor and supports counter-propagating illuminating and reflecting optical signals.

Another method of detection involves the use of a beam splitter that enables a single illuminating fiber, which is connected to a light source, to be oriented at a 90 degree angle to a collecting fiber, which is connected to a detector. Light is directed through the illuminating fiber probe into the beam splitter, which directs light at the biosensor. The reflected light is directed back into the beam splitter, which directs light into the collecting fiber probe. A beam splitter allows the illuminating light and the reflected light to share a common optical path between the beam splitter and the biosensor, so perfectly collimated light can be used without defocusing.

### Surface of Biosensor

A ligand or specific binding substance is a molecule that binds to another molecule. Ligand and specific binding substance are analogous terms. A ligand or specific binding substance can be, for example, a nucleic acid, peptide, extracellular matrix ligand (see Table 1), protein solutions, peptide solutions, single or double stranded DNA solutions, RNA solutions, RNA-DNA hybrid solutions, solutions containing compounds from a combinatorial chemical library, antigen, polyclonal antibody, monoclonal antibody, single chain antibody (scFv), F(ab) fragment, F(ab′)₂ fragment, Fv fragment, small organic molecule, cell, virus, bacteria, polymer or biological sample. A biological sample can be for example, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, or prostatic fluid. The polymer is selected from the group of long chain molecules with multiple active sites per molecule consisting of hydrogel, dextran, poly-amino acids and derivatives thereof, including poly-lysine (comprising poly-l-lysine and poly-d-lysine), poly-phe-lysine and poly-glu-lysine. In one embodiment of the invention, ligands are extracellular matrix protein ligands.

Binding partners are, for example, added to a biosensor surface comprising specific binding substances, ligands or cells to determine, e.g., if the binding partners bind to the specific binding substances, ligands or cells or change the specific binding substances, ligands or cells in any manner (e.g., cause a cell to differentiate or de-differentiate). Binding partners can be, e.g., a nucleic acid, peptide, extracellular matrix ligand (see Table 1), protein solutions, peptide solutions, single or double stranded DNA solutions, RNA solutions, RNA-DNA hybrid solutions, solutions containing compounds from a combinatorial chemical library, antigen, polyclonal antibody, monoclonal antibody, single chain antibody (scFv), F(ab) fragment, F(ab′)₂ fragment, Fv fragment, small organic molecule, cell, virus, bacteria, polymer or biological sample. A biological sample can be for example, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, or prostatic fluid. The polymer is selected from the group of long chain molecules with multiple active sites per molecule consisting of hydrogel, dextran, poly-amino acids and derivatives thereof, including poly-lysine (comprising poly-l-lysine and poly-d-lysine), poly-phe-lysine and poly-glu-lysine.

Immobilization of one or more ligands onto a biosensor is performed so that a ligand will not be washed away by any rinsing procedures, and so that the binding of the ligand to binding partners in a test sample is unimpeded by the biosensor surface. One or more ligands can be attached to a biosensor surface by physical adsorption (i.e., without the use of chemical linkers) or by chemical binding (i.e., with the use of chemical linkers) as well as electrochemical binding, electrostatic binding, hydrophobic binding and hydrophilic binding. Chemical binding can generate stronger attachment of ligands on a biosensor surface and provide defined orientation and conformation of the surface-bound molecules. In one embodiment of the invention a ligand or specific binding substance can become associated with a biosensor surface such that it is not immobilized but remains associated with the biosensor surface due to gravity or a gel or gel-like substance that is added over the ligand or specific binding substance.

A ligand or specific binding substance can also be specifically bound to a biosensor surface via a specific binding substance such as a nucleic acid, peptide, protein solution, peptide solution, solutions containing compounds from a combinatorial chemical library, antigen, polyclonal antibody, monoclonal antibody, single chain antibody (scFv), F(ab) fragment, F(ab′)₂ fragment, Fv fragment, small organic molecule, virus, polymer or biological sample, wherein the specific binding substance is immobilized to the surface of the biosensor.

Furthermore, ligands or specific binding substances can be arranged in an array of one or more distinct locations on the biosensor surface, wherein the surface can reside within one or more wells of a multiwell plate and comprising one or more surfaces of the multiwell plate or microarray. The array of ligands comprises one or more ligands on the biosensor surface within a microwell plate such that a surface contains one or more distinct locations, each with a different ligand. For example, an array can comprise 1, 10, 100, 1,000, 10,000 or 100,000 or greater distinct locations. Thus, each well of a multiwell plate or microarray can have within it an array of one or more distinct locations separate from the other wells of the multiwell plate, which allows multiple different samples to be processed on one multiwell plate. The array or arrays within any one well can be the same or different than the array or arrays found in any other microtiter wells of the same microtiter plate. Additionally, an array of the invention can comprise one or more specific binding substances in any type of regular or irregular pattern. For example distinct locations can define an array of spots of one or more binding substances. An array spot can be about 10, 20, 30, 40, 50, 100, 200, 300, 400, or 500 microns in diameter.

A specific binding substance specifically binds to a binding partner (i.e., a cell or molecule on the cell) that is added to the surface of a biosensor of the invention such that the cell becomes immobilized to the biosensor. A specific binding substance specifically binds to its binding partner, but does not substantially bind other binding partners added to the surface of a biosensor. For example, where the specific binding substance is an antibody and its binding partner is a particular antigen, the antibody specifically binds to the particular antigen, but does not substantially bind other antigens. A binding partner can be, for example, a cell or any molecule present on or within cell such as a nucleic acid, a recombinant nucleic acid, a protein, a recombinant protein, an extracellular matrix protein receptor, a lipid, or a carbohydrate. In one embodiment of the invention a binding partner is a receptor that can bind a specific binding substance immobilized on the biosensor, wherein the receptor is on the surface of a cell.

While microtiter plates are the most common format used for biochemical assays, microarrays are increasingly seen as a means for maximizing the number of biochemical interactions that can be measured at one time while minimizing the volume of precious reagents. By application of specific binding substances with a microarray spotter onto one biosensor surface of the invention, specific binding substance densities of 10,000 specific binding substances/in² can be obtained. By focusing an illumination beam to interrogate a single microarray location, a biosensor can be used as a label-free microarray readout system.

Immobilization of a ligand to a biosensor surface can be also be affected via binding to, for example, the following functional linkers: a nickel group, an amine group, an aldehyde group, an acid group, an alkane group, an alkene group, an alkyne group, an aromatic group, an alcohol group, an ether group, a ketone group, an ester group, an amide group, an amino acid group, a nitro group, a nitrile group, a carbohydrate group, a thiol group, an organic phosphate group, a lipid group, a phospholipid group or a steroid group. Furthermore, a ligand can be immobilized on the surface of a biosensor via physical adsorption, chemical binding, electrochemical binding, electrostatic binding, hydrophobic binding or hydrophilic binding, and immunocapture methods.

In one embodiment of the invention a biosensor can be coated with a linker such as, e.g., a nickel group, an amine group, an aldehyde group, an acid group, an alkane group, an alkene group, an alkyne group, an aromatic group, an alcohol group, an ether group, a ketone group, an ester group, an amide group, an amino acid group, a nitro group, a nitrile group, a carbohydrate group, a thiol group, an organic phosphate group, a lipid group, a phospholipid group or a steroid group. For example, an amine surface can be used to attach several types of linker molecules while an aldehyde surface can be used to bind proteins directly, without an additional linker. A nickel surface can be used to bind molecules that have an incorporated histidine (“his”) tag. Detection of “his-tagged” molecules with a nickel-activated surface is well known in the art (Whitesides, Anal. Chem. 68, 490, (1996)).

Linkers, ligands, and specific binding substances can be immobilized on the surface of a biosensor such that each well has the same linker, ligands, and/or specific binding substances immobilized therein. Alternatively, each well can contain a different combination of linkers, ligands, and/or specific binding substances.

A ligand or specific binding substance can specifically or non-specifically bind to a linker immobilized on the surface of a biosensor. Alternatively, the surface of the biosensor can have no linker and a ligand or specific binding substance can bind to the biosensor surface non-specifically.

Immobilization of one or more specific binding substances or linkers onto a biosensor is performed so that a specific binding substance or linker will not be washed away by rinsing procedures, and so that its binding to ligand in a test sample is unimpeded by the biosensor surface. Several different types of surface chemistry strategies have been implemented for covalent attachment of specific binding substances to, for example, glass for use in various types of microarrays and biosensors. These same methods can be readily adapted to a biosensor of the invention. Surface preparation of a biosensor so that it contains the correct functional groups for binding one or more specific binding substances is an integral part of the biosensor manufacturing process.

One or more specific ligands or specific binding substances can be attached to a biosensor surface by physical adsorption (i.e., without the use of chemical linkers) or by chemical binding (i.e., with the use of chemical linkers) as well as electrochemical binding, electrostatic binding, hydrophobic binding and hydrophilic binding. Chemical binding can generate stronger attachment of ligands on a biosensor surface and provide defined orientation and conformation of the surface-bound molecules.

Immobilization of ligands to plastic, epoxy, or high refractive index material can be performed essentially as described for immobilization to glass. However, the acid wash step can be eliminated where such a treatment would damage the material to which the specific binding substances are immobilized.

Cells such as primary cells or stem cells can be immobilized to the biosensor by one or more ligands or ligands. In one embodiment of the invention, cells are immobilized to the biosensor through a reaction with extracellular matrix ligands. Integrins are cell surface receptors that interact with the extracellular matrix (ECM) and mediate intracellular signals. Integrins are responsible for cytoskeletal organization, cellular motility, regulation of the cell cycle, regulation of cellular of integrin affinity, attachment of cells to viruses, attachment of cells to other cells or ECM. Integrins are also responsible for signal transduction, a process whereby the cell transforms one kind of signal or stimulus into another intracellularly and intercellularly. Integrins can transduce information from the ECM to the cell and information from the cell to other cells (e.g., via integrins on the other cells) or to the ECM. A list of integrins and their ECM ligands can be found in Takada et al. Genome Biology 8:215 (2007) as shown in Table 1.

TABLE 1

| Integrin | ECM ligand |
|---|---|
| α₁β₁ | Laminin, collagen |
| α₂β₁ | Laminin, collagen, thrombospondin, E- |
| | cadherin, tenascin |
| α₃β₁ | Laminin, thrombospondin, uPAR |
| α₄β₁ | Thrombospondin, MadCAM-1, VCAM-1, |
| | fibronectin, osteopontin, ADAM, ICAM-4 |
| α₅β₁ | Fibronectin, osteopontin, fibrillin, |
| | thrombospondin, ADAM, COMP, L1 |
| α₆β₁ | Laminin, thrombospondin, ADAM, Cyr61 |
| α₇β₁ | Laminin |
| α₈β₁ | Tenascin, fibronectin, osteopontin, |
| | vitronectin, LAP-TGF- β, nephronectin, |
| α₉β₁ | Tenascin, VCAM-1, osteopontin, uPAR, |
| | plasmin, angiostatin, ADAM, VEGF-C, |
| | VEGF-D |
| α₁₀β₁ | Laminin, collegen |
| α₁₁β₁ | Collagen |
| αvβ₁ | LAP-TGF- β, fibronectin, osteopontin, |
| | L1 |
| αLβ₂ | ICAM, ICAM-4 |
| αMβ₂ | ICAM, iC3b, factor X, fibrinogen, ICAM- |
| | 4, heparin |
| αXβ₂ | ICAM, iC3b, fibrinogen, ICAM-4, |
| | heparin, collagen |
| αDβ₂ | ICAM, VCAM-1, fibrinogen, fibronectin, |
| | vitronectin, Cyr61, plasminogen |
| α_{IIb}β₃ | Fibrinogen, thrombospondin, fibronectin, |
| | vitronectin, vWF, Cyr61, ICAM-4, L1, |
| | CD40 ligand |
| α_{V}β₃ | Fibrinogen, vitronectin, vWF, |
| | thrombospondin, fibrillin, tenascin, |
| | PECAM-1, fibronectin, osteopontin, |
| | BSP, MFG-E8, ADAM-15, COMP, |
| | Cyr61, ICAM-4, MMP, FGF-2, uPA, |
| | uPAR. L1, angiostatin, plasmin, |
| | cardiotoxin, LAP-TGF- β, Del-1 |
| α6β₄ | Laminin |
| α_{V}β₅ | Osteopontin, BSP, vitronectin, CCN3 |
| | [35], LAP-TGF- β |
| α_{V}β₆ | LAP-TGF- β, fibronectin, osteopontin, |
| | ADAM |
| α₄β₇ | MAdCAM-1, VCAM-1, fibronectin, |
| | osteopontin |
| αEβ₇ | E-cadherin |
| αvβ₈ | LAP-TGF- β |

Abbreviations: ADAM, a disintegrin metalloprotease; BSP, bone sialic protein; CCN3, an extracellular matrix protein; COMP, cartilage oligomeric matrix protein, Cyr61, cysteine-rich protein 61; L1, CD171; LAP-TGF- β latency-associated peptide; iC3b, inactivated complement component 3; PECAM-1, platelet and endothelial cell adhesion molecule 1; uPA, urokinase; uPAR, urokinase receptor; VEGF, vascular endothelial growth factor; vWF, von Willebrand Factor.

Other cell surface receptors that interact with the ECM include focal adhesion proteins. Focal adhesion proteins form large complexes that connect the cytoskeleton of a cell to the ECM. Focal adhesion proteins include, for example, talin, α-actinin, filamin, vinculin, focal adhesion kinase, paxilin, parvin, actopaxin, nexilin, fimbrin, G-actin, vimentin, syntenin, and many others.

Yet other cell surface receptors can include, but are not limited to those that can interact with the ECM include cluster of differentiation (CD) molecules. CD molecules act in a variety of ways and often act as receptors or ligands for the cell. Other cell surface receptors that interact with ECM include cadherins, adherins, and selectins.

The ECM has many functions including providing support and anchorage for cells, segregation of tissue from one another, and regulation of intracellular communications. The ECM is composed of fibrous proteins and glycosaminoglycans. Glycosaminoglycans are carbohydrate polymers that are usually attached to the ECM proteins to form proteoglycans (including, e.g., heparin sulfate proteoglycans, chondroitin sulfate proteoglycans, karatin sulfate proteoglycans). A glycosaminoglycan that is not found as a proteoglycan is hyaluronic acid. ECM proteins include, for example, collagen (including fibrillar, facit, short chain, basement membrane and other forms of collagen), fibronectin, elastin, and laminin (see Table 1 for additional examples of ECM proteins). ECM ligands useful herein include ECM proteins, glycosaminoglycans, proteoglycans, and hyaluronic acid.

“Specifically binds,” “specifically bind” or “specific for” means that a cell surface receptor, e.g., an integrin or focal adhesion protein, etc., binds to a cognate extracellular matrix ligand, with greater affinity than to other, non-specific molecules. A non-specific molecule does not substantially bind to the cell receptor. For example, the integrin α4/β1 specifically binds the ECM ligand fibronectin, but does not specifically bind the non-specific ECM ligands collagen or laminin. In one embodiment of the invention, specific binding of a cell surface receptor to an extracellular matrix ligand, wherein the extracellular matrix ligand is immobilized to a surface, e.g., a biosensor surface, will immobilize the cell to the extracellular matrix ligand and therefore to the surface such that the cell is not washed from the surface by normal cell assay washing procedures.

By specifically immobilizing cells to a biosensor surface through binding of cell surface receptors, such as integrins, to ECM ligands, antibodies, cognate binding proteins, or peptide mimetics that are immobilized to the biosensor, the binding of the cells to the biosensor and the cells' response to stimuli is dramatically altered as compared to cells that are non-specifically immobilized (i.e., immobilization of all cells in general instead of immobilizing certain cells through specific binding reactions, e.g., the binding of cell surface receptor to an antibody that specifically binds the cell surface receptor) to a biosensor surface. That is, detection of response of cells to stimuli is greatly enhanced or augmented where cells are immobilized to a biosensor via ECM ligand binding. In one embodiment of the invention, the cells can be in a serum-free medium when they are added to the biosensor surface. A serum-free medium contains about 10, 5, 4, 3, 2, 1, 0.5% or less serum. A serum-free medium can comprise about 0% serum or about 0% to about 1% serum. In one embodiment of the invention cells are added to a biosensor surface where one or more types of ECM ligands have been immobilized to the biosensor surface. In another embodiment of the invention, cells are combined with one or more types of ECM ligands and then added to the surface of a biosensor.

In one embodiment of the invention, an ECM ligand is purified. A purified ECM ligand is an ECM ligand preparation that is substantially free of cellular material, other types of ECM ligands, chemical precursors, chemicals used in preparation of the ECM ligand, or combinations thereof. An ECM ligand preparation that is substantially free of other types of ECM ligands, cellular material, culture medium, chemical precursors, chemicals used in preparation of the ECM ligand, etc., has less than about 30%, 20%, 10%, 5%, 1% or more of other ECM ligands, culture medium, chemical precursors, and/or other chemicals used in preparation. Therefore, a purified ECM ligand is about 70%, 80%, 90%, 95%, 99% or more pure. A purified ECM ligand does not include unpurified or semi-purified preparations or mixtures of ECM ligands that are less than 70% pure, e.g., fetal bovine serum. In one embodiment of the invention, ECM ligands are not purified and comprise a mixture of ECM proteins and non-ECM proteins. Examples of non-purified ECM ligand preparations include fetal bovine serum, bovine serum albumin, and ovalbumin.

For example, cells expressing α4/β1 integrin receptors, which are known to bind to fibronectin ligands, but not to collagen or laminin ligands, generate a PWV shift on fibronectin coated wells that is about 8 to 10 times greater than the PWV shift observed on collagen or laminin surfaces. PWV shifts for cells expressing α4/β1 integrin receptors on biosensor surfaces having collagen or laminin immobilized to them resembles background cell attachment signal observed on BSA-coated control wells.

In one embodiment of the invention detection of cell binding to ECM ligands is increased by about 2, 3, 4, 5, 6, 7, 8, 9, 10, 15, 20 or more times (or any range between 2 and 20 times) when the ECM ligand is specific for a cell surface receptor, e.g., an integrin or focal adhesion protein, present on the surface of the cells. In another embodiment of the invention detection of cellular responses to stimuli is increased by about 2, 3, 4, 5, 6, 7, 8, 9, 10, 15, 20 or more times (or any range between 2 and 20 times) when the cell is immobilized to the biosensor surface by an ECM ligand that is specific for a cell surface receptor, e.g., an integrin.

Once cells are attached to the biosensor through ligands, ECM, or other means one or more ligands can be added to the cells to determine the reaction of the cell to the one or more ligands.

### Liquid-Containing Vessels

A biosensor can comprise an inner surface, for example, a bottom surface of a liquid-containing vessel. A liquid-containing vessel can be, for example, a microtiter plate well, a test tube, a petri dish, microarray slide, microscope slide, a biosensor surface, or a microfluidic channel. One embodiment of this invention is a biosensor that is incorporated into any type of microtiter plate. For example, a biosensor can be incorporated into the bottom surface of a microtiter plate by assembling the walls of the reaction vessels over the biosensor surface, so that each reaction “spot” can be exposed to a distinct test sample. Therefore, each individual microtiter plate well can act as a separate reaction vessel. Separate chemical reactions can, therefore, occur within each individual vessel, such as adjacent wells without intermixing reaction fluids and chemically distinct test solutions can be applied to individual vessels.

Several methods for attaching a biosensor or grating of the invention to the bottom surface of bottomless microtiter plates can be used, including, for example, adhesive attachment, ultrasonic welding, and laser welding.

The most common assay formats for pharmaceutical high-throughput screening laboratories, molecular biology research laboratories, and diagnostic assay laboratories are microtiter plates. The plates are standard-sized plastic cartridges that can contain about 2, 6, 8, 24, 48, 96, 384, 1536, 3456, 9600 or more individual reaction vessels arranged in a grid. Due to the standard mechanical configuration of these plates, liquid dispensing, robotic plate handling, and detection systems are designed to work with this common format. A biosensor of the invention can be incorporated into the bottom surface of a standard microtiter plate. Because the biosensor surface can be fabricated in large areas, and because the readout system does not make physical contact with the biosensor surface, an arbitrary number of individual biosensor areas can be defined that are only limited by the focus resolution of the illumination optics and the x-y stage that scans the illumination/detection probe across the biosensor surface.

### Method of Using Biosensors

Biosensors of the invention can be used to study one or a number of specific binding substance/ligand and binding partner interactions in parallel. Binding of one or more specific binding substances or ligands to their respective binding partners can be detected, without the use of labels, by applying one or more binding partners (e.g., cells bearing receptors or antigens or other molecules that bind to specific binding substances) to a biosensor surface that has one or more specific binding substances immobilized to its surface at individual distinct locations. In one embodiment of the invention, one or more specific binding substances are one or more extracellular matrix protein ligands and the one or more binding partners are receptors on cells, wherein the receptors (e.g., an integrin) are specific for extracellular matrix protein ligands. A biosensor is illuminated with light and a maxima in reflected wavelength, or a minima in transmitted wavelength of light is detected from the biosensor for each distinct location. Signals are detected from a grating-based waveguide biosensor and are compared to each other or to controls in a manner similar to that for colorimetric resonant reflectance biosensors. All assays or methods described herein can be performed on colorimetric resonant reflectance biosensors, diffraction anomaly biosensors, diffraction grating biosensors, dielectric stack biosensors, and grating-based waveguide biosensors. If one or more specific binding substances have bound to their respective binding partners on a colorimetric resonant reflectance biosensor, then the reflected wavelength of light is shifted at that distinct location as compared to a situation where one or more specific binding substances have not bound to their respective binding partners. Where a biosensor is coated with an array of one or more distinct locations containing the one or more specific binding substances, then a maxima in reflected wavelength or minima in transmitted wavelength of light is detected from each distinct location of the biosensor. Where one or more specific binding substances have bound to their respective binding partners on a grating based biosensor a change in effective refractive index occurs.

In one embodiment of the invention, a variety of specific binding substances, for example, specific binding substances specific for cell receptors or cell antigens, specific for proteins expressed, down-regulated, or up-regulated on a cell surface when the cell is infected with one or more viruses (see, Liang et al., Proc. Natl. Acad. Sci. USA (2005) 102:5838), or specific for proteins expressed by a cell that are associated with apoptosis (e.g., the up-regulation of p53, TNF-α, TNF-β, Fas ligand; the down-regulation of growth factors for neurons and IL-2), can be immobilized in an array format onto a biosensor of the invention. The biosensor is then contacted with a test sample of interest comprising binding partners, such as cells bearing ECM ligand receptors, e.g., integrins or focal adhesion proteins. Only the cells that specifically bind to the specific binding substances are immobilized on the biosensor surface. In one embodiment of the invention, cells that are bound through ECM ligands can respond to stimuli unlike unbound cells. The use of a detectable label, such as an enzyme label, a radioactive label, or a fluorescent label, is not required to detect the response of the cells to stimuli, test reagents, or incubation time. For high-throughput applications, biosensors can be arranged in an array of arrays, wherein several biosensors comprising an array of specific binding substances are arranged in an array. Such an array of arrays can be, for example, dipped into microtiter plate to perform many assays at one time. In another embodiment, a biosensor can occur on the tip of a fiber probe for in vivo detection of biochemical substance. Alternatively, cells can be mixed with ECM ligands or be derived as a mixture of cells and ECM and then added to a biosensor surface.

The cells added to the biosensor can be prokaryotic cells, such as bacteria or archaea or eukaryotic cells such as animal, fungi, plant, and protist cells. Cells can be mammalian cells such as human cells. Any amount of cells can be added to a biosensor of the invention. For example, about 1, 2, 3, 4, 5, 10, 15, 50, 100, 150, 200, 300, 500, 1,000, 10,000, 100,000 or more cells (or any range or value between about 1 and 100,000; for example from about 50 to about 100, about 50 to about 200, about 50 to about 500, about 50 to about 1,000) can be used in an assay of the invention.

One embodiment of the invention allows the direct detection of cell changes, such as changes in cell growth patterns, up- or down-regulation or expression of an analyte, such as a cell surface receptor, by a cell (e.g., increase or decrease in cell receptor or analyte expression or changes over time in cell receptor or analyte expression in response to certain stimuli (e.g., an increase in expression of a cell receptor when the cell is immobilized and incubated on a biosensor surface followed by a decrease in cell receptor expression when stimuli is added to the cell)), cell death patterns, changes in cell differentiation, changes in cell movement, changes in cell size or volume, or changes in cell adhesion, as they occur in real time with a colorimetric resonant reflectance biosensor or grating based waveguide biosensor and without the need to incorporate or without interference from radiometric, colorimetric, or fluorescent labels (although labels may be used if desired). Changes in cell behavior and morphology can be detected as the cell is perturbed. The cellular changes can then be detected in real time using a high speed, high resolution instrument, such as the BIND® READER (i.e., a colorimetric resonant reflectance biosensor system), and corresponding algorithms to quantify data. See, e.g., U.S. Pat. Nos. 7,422,891; 7,327,454, 7,301,628, 7,292,336; 7,170,599; 7,158,230; 7,142,296; 7,118,710. By combining this methodology, instrumentation and computational analysis, cellular behavior can be expediently monitored in real time (i.e., expediently and conveniently observing and quantifying cell reactions during the instant the cell is responding to stimulus or test reagent and over time while the cell is responding to the stimulus or test reagent), in a label free manner. A label-free manner means that the cells do not have labels (e.g., a fluorescent label, a radioactive label, an enzymatic label, affinants for labels, a magnetic label, a chemiluminescent label, a luminescent label, a bioluminescent label, a chemical label etc.) that are attached or associated with the cells and that are used to detect cells or changes to the cells. Detectable labels (e.g., a fluorescent label, a radioactive label, an enzymatic label, affinants for labels, a magnetic label, a chemiluminescent label, a luminescent label, a bioluminescent label, a chemical label etc.) are attached or associated with cells and are used to detect cells or changes in cells. Real time monitoring occurs when multiple readings (e.g., every about 0.001, 0.01, 0.1, 1.0, 5, 10, 20, 30, 40, 50, or 60 seconds, every about 1, 2, 3, 4, 5, 10, 20, 30, 45, or 60 minutes, every about 1, 2, 6, 12, or 24 hours) are taken from the biosensor surface over the entire course of the assay (e.g., about 1, 2, 3, 4, 5, 10, 20, 30, 45, or 60 minutes or about 1, 2, 3, 4, 5, 10, 12, 24, or 48 hours, or about 1, 2, 3, 4, 5, 10, 20 or 30 days, depending on the type of assay).

Colorimetric resonant reflectance biosensors, such as SRU Biosystems, Inc. BIND™ technology (Woburn, Mass.) have the capability of measuring changes to a surface with respect to mass attachment from nanoscale biological systems. The applications and the methods, in which colorimetric resonant reflectance biosensors have been previously implemented, have changed as the resolution of the instruments has improved. Previously, measurement of the quantity of cells attached to the colorimetric resonant reflectance biosensor surface was the primary goal. While looking at some poorer resolution images of cells, however, it was noted that cells gave differential signals with respect to the number of pixels occupied, intensity of signal/pixel, change in PWV of each pixel, etc. While trying to reduce the variability of these data, it became clear that the variability lay within the individual cells and their differential morphological responses to stimuli. To further investigate these cellular events, a higher resolution version of a BIND® READER (i.e., a colorimetric resonant reflectance biosensor system), the BIND® SCANNER (a high resolution colorimetric resonant reflectance biosensor system) was constructed. See, e.g., U.S. Pat. Nos. 7,301,628; 7,298,477; 7,148,964; 7,023,544.

A BIND® SCANNER (i.e., a high resolution colorimetric resonant reflectance biosensor system) has a high resolution lens. The lens has a resolution of about 2, 3, 3.75, 4, 5, 6, 7, 8, 9, 10, 12, 15, 20, 50, 100, 200, 500, 1,000, or 2,000 micrometers (or any range between about 2 and about 2,000 micrometers, for example: 2-5, 2-3.75, 2-10, 2-15, 8-12, 2-20, 2-50, 2-100, 2-200 or 2-300 micrometers). Additionally, methodologies were developed for analyzing cell changes in real time at better resolution. The advantage of the BIND® SCANNER's high resolution is that it allows the analysis of wavelength shifts at different pixel locations within a single well or vessel. A whole biosensor microtiter well can be read by the scanner or only a small portion of the well or surface.

Methods of the invention can be used to detect cell changes including changes in cell growth patterns or expression of cell receptors or analytes. Briefly, cells can be immobilized on a colorimetric resonant reflectance optical biosensor; a PWV is detected; the cells are subjected to a test reagent, an incubation, or stimuli; a PWV is detected; and the initial PWV and the subsequent PWV can be compared, wherein the difference between the initial PWV in relation to the subsequent PWV indicates a change in cell growth pattern or other cell changes. Optionally, changes in PWV can also be determined and recorded at several time points during the course of the assay and compared.

The change in cell growth pattern can be selected from the group consisting of cell morphology, cell adhesion, cell migration, cell proliferation and cell death. One type of prokaryotic or eukaryotic cells or two or more types of eukaryotic or prokaryotic cells can be immobilized on the biosensor.

The methods of the invention provide unique opportunities to detect changes in cells, such as primary cells and stem cells, including, e.g., chemotaxis assays, low cell number assays, differentiation assays, migration assays, attachment assays, cell invasion assays, adhesion assays, biological profiling of differentiated states of cells.

Biosensor systems of the invention are also capable of detecting and quantifying the amount of a binding partner from a sample that is bound to one or more distinct locations defining an array by measuring the shift in reflected wavelength of light. For example, the wavelength shift at one or more distinct locations can be compared to positive and negative controls at other distinct locations to determine the amount of a specific binding substance that is bound. Importantly, numerous such one or more distinct locations can be arranged on the biosensor surface, and the biosensor can comprise an internal surface of a vessel such as an about 2, 6, 8, 24, 48, 96, 384, 1536, 3456, 9600 or more well-microtiter plate. As an example, where 96 biosensors are attached to a holding fixture and each biosensor comprises about 100 distinct locations, about 9600 biochemical assays can be performed simultaneously.

### Methods of Sorting, Analyzing and Quantifying Cells

Methods of the invention provide methods of sorting 1, 2, 3, 4, 5, 6, 7, 8, 9, 10, 15, 20 or more cell types from a mixed population of cells and detecting the response of the sorted cells to stimuli, incubation, or test reagents, wherein the sorting and the detection occur on one biosensor surface. A mixed population of cells is applied to one colorimetric resonant reflectance biosensor surface or other biosensor surface, wherein the biosensor has one or more specific binding substances (e.g., an antibody or ECM ligand) immobilized to its one surface, wherein the one or more specific binding substances can potentially bind one or more cell types in the mixed population of cells. Optionally, unbound cells are washed from the surface of the biosensor, such that one or more cell types are bound to and sorted on the surface of the biosensor. The one or more bound cell types are exposed to stimuli, test reagents, incubations or combinations thereof. The response of the one or more bound cell types to the stimuli is detected by detecting a PWV shift or change in effective refractive index. The PWVs and effective refractive indices can be compared over time, compared in real time, or can be compared to negative or positive controls. Therefore, one surface of a biosensor can be used to sort, detect, quantify and/or analyze the response of one or more cell types in a mixed population to stimuli, test reagents, incubations or combinations thereof.

Sorting of cells can be the immobilization of less than all cell types of a mixed population sample onto a biosensor surface, wherein the non-immobilized cells of the sample are optionally washed away. Sorting cells can also refer to the immobilization of one cell type to one distinct location on a biosensor while one or more other cell types are immobilized to other distinct locations on the biosensor surface. Non-immobilized cells can optionally be washed away or can remain on the biosensor surface.

The methods of the invention also provide methods of sorting one, two, or more cell types from a mixed population of cells and detecting an intracellular analyte of the cells or other analyte produced by the one or more cell types on one biosensor surface. In one embodiment of the invention a mixed population of cells is applied to one colorimetric resonant reflectance biosensor surface or one grating-based waveguide biosensor surface. The one biosensor surface can have two or more specific binding substances immobilized to its one surface, wherein the two or more specific binding substances comprise (i) first specific binding substances that specifically bind one or more cell types in the mixed population of cells and (ii) second specific binding substances that specifically bind one or more intracellular analytes from the one or more cell types. The first and second specific binding substances can be different or the same. Optionally, the unbound cells can be washed from the surface of the biosensor, such that the one or more cell types are bound to and sorted on the surface of the biosensor. The one or more bound cell types are lysed or permeabilized with, e.g., biological detergents, TWEEN®, TRITON®, NP40, Brij, octyl-beta-thioglucopyranoside, digitonin, formaldehyde, paraformaldehyde, high concentrations of salt, or combinations thereof. Alternatively, the cells can be incubated for a period time or exposed to stimuli and then optionally incubated prior to lysis. After lysis, permeablization, incubation, exposure to stimuli (or any combination thereof) any unbound analytes can optionally be washed from the surface of the biosensor. The intracellular analytes immobilized to the surface of the biosensor are detected by detecting a PWV shift or change in effective refractive index at each distinct location of the biosensor. The PWVs and effective refractive indices can be compared over time or can be compared to negative or positive controls. Therefore, a mixed population cell sample can be used to sort, detect, quantify, and/or analyze an intracellular component of one or more specific types of cells within the mixed population cell sample. Intracellular analytes or other analytes can be, e.g., proteins, RNA, DNA, carbohydrates, lipids, cell receptors, or any other molecule that would be present on or within a cell or produced by a cell.

In another embodiment of the invention, one, two, or more cell types can be sorted from a mixed population of cells and an analyte from the one or more cell types can be detected using only one biosensor surface. A mixed population of cells is applied to one colorimetric resonant reflectance biosensor surface or one grating-based waveguide biosensor surface. The one biosensor surface has two or more specific binding substances immobilized to its one surface, wherein the two or more specific binding substances comprise (i) first specific binding substances that specifically bind one or more cell types in the mixed population of cells and (ii) second specific binding substances that specifically bind one or more analytes from the one or more cell types. The unbound cells are optionally washed from the surface of the biosensor, such that the one or more cell types are bound to and sorted on the surface of the biosensor. The cells are contacted with a test reagent, or are incubated, or subjected to stimuli or a combination thereof. The analytes immobilized to the surface of the biosensor are detected. The analytes immobilized to the surface of the biosensor are detected by detecting a PWV shift or change in effective refractive index. The PWVs and effective refractive indices can be compared over time or can be compared to negative or positive controls. Analytes can be, e.g., e.g., proteins, RNA, DNA, carbohydrates, lipids, or any other molecule that can be produced by a cell in response to an incubation, test reagents, or exposure to stimuli.

Where one or more specific binding substances that specifically bind one or more cell types and one or more specific binding substances that specifically bind one or more intracellular analytes or other analyte from the one or more cell types are immobilized to a surface of a biosensor the specific binding substances that specifically bind one or more cell types can be in one distinct location on the one biosensor surface and the one or more specific binding substances that specifically bind one or more intracellular analytes or other analyte from the one or more cell types can be present in a second distinct location. Each one or more specific binding substances that specifically bind one or more cell types and one or more specific binding substances that specifically bind one or more intracellular analytes or other analyte from the one or more cell types can be present at its own distinct location on the one biosensor surface. Alternatively, the different types of specific binding substances can be present or mixed together at one distinct location on the one biosensor surface. One biosensor surface and one distinct location on a biosensor surface can comprise 1, 2, 3, 4, 5, 6, 7, 8, 9, 10, 20, 30 or more specific binding substance types.

One biosensor surface can comprise 1, 2, 3, 4, 5, 6, 7, 8, 9, 10, 20, 30, 40, 50, 100, 500, 1,000 or more distinct locations. Each distinct location can have 1, 2, 3, 4, 5, 6, 7, 8, 9, 10, 20, 30, 40, 50, 100 or more specific binding substances immobilized thereon. For example, one biosensor surface can have two distinct locations. At the first distinct location one specific binding substance type can be immobilized. At the second distinct location two specific binding substances of different types from the other specific binding substances can be immobilized.

The methods of the invention can also be used to sort two or more types of cells (e.g., 2, 3, 4, 5, 10, 15, 20 or more types of cells) from a mixed population of cells into two or more distinct locations on one biosensor surface. For example, a mixed population of cells containing, e.g., greater than 2, 3, 4, 5, 10, 15, 20 or 30 cell types can be added to one biosensor surface having two or more types of specific binding substances (e.g., about 2, 3, 4, 5, 10, 15, 20 or more) immobilized in two or more distinct locations. The two or more specific binding substances can bind to and immobilize two or more types of cells from the mixed population of cells. Therefore, cells will be sorted into two or more distinct locations on one surface of a biosensor. Unbound cells from the mixed population of cells can be washed away. The cells can then be stimulated, subjected to test reagents, lysed, or permeabilized. Detection, enumeration and analysis can performed at each step of the assay.

One embodiment of the invention provides methods to quantify the number or amount of binding partners, e.g., cell receptors or cell surface antigens, on cells that specifically bind to specific binding substances that are immobilized on the one biosensor surface. A mixed population of cells is applied to one colorimetric resonant reflectance biosensor surface or one grating-based waveguide biosensor surface. The one biosensor surface has one or more specific binding substances immobilized to its one surface, wherein the one or more specific binding substances specifically bind one or more binding partners, e.g., a cell receptor or other protein or analyte on the cell surface, on a cell in the mixed population of cells. The unbound cells are optionally washed from the surface of the biosensor, such that the one or more cell types are bound to and sorted on the surface of the biosensor. The cells are optionally contacted with a test reagent, or are incubated, or subjected to stimuli or a combination thereof. The amount of cells or cell receptors bound to the surface of the biosensor is analyzed by detecting a PWV shift or change in effective refractive index. The PWVs and effective refractive indices can be compared over time or can be compared to negative or positive controls. The amount of binding partners on the cells can be determined by comparisons to, e.g., control values. Control values can be derived from cells comprising known numbers of cell receptors or cell surface antigens.

For all assays described herein PWVs and effective refractive index readings can be taken before each wash or addition to the biosensor surface, during each addition to the biosensor surface, after each wash or addition to the biosensor surface, before or after each incubation period, or a combination thereof. PWVs or effective refractive index readings can also be taken continuously over the course of the assay in real time.

A mixed population of cells or “two or more cells” comprises about 2, 3, 4, 5, 10, 15, 20, 30 or more different types of cells. A mixed population of cells (or “two or more cells”) can comprise any mixture of different types of cells, e.g., a mixture of red blood cells, leukocytes, and platelets; a mixture of different types of bacteria; a mixture of different types of cells present in a biological sample; a mixture of stem cells; and a mixture of differentiated cell types. Stem cell populations can be considered to be a mixed population of cells because the cells in a stem cell population are often present at different stages of differentiation. A mixed population of cells can be, e.g., lung aspirate, sputum, saliva, blood, plasma, tissue, feces, urine, bone marrow, lymph nodes, environmental samples, food samples. The mixed population of cells can be partially purified, unpurified, concentrated, unconcentrated, or undiluted. Samples, such as tissue samples or fecal samples, can be broken up and suspended in buffer prior to use. A mixed population of cells can be biopsy material that would be expected to comprise about 2, 3, 4, 5, 10, 15, 20, 30 or more types of cells. A biopsy can include tissue collected by a fine needle aspiration, core needle biopsy, vacuum assisted biopsy, open surgical biopsy, skin biopsy (e.g., shave, punch, incisional, excisional, or curettage). A biopsy can collect, e.g., bone marrow, endometrial, skin, lymph node, liver, lung, gastrointestinal tract, kidney, transplanted organ, or testicular tissue. In general, a mixed population of cells contains two or more cell types that potentially bind to specific binding substances immobilized to the surface of a biosensor. That is, out of the mixed population of cells only a subset of the cells (i.e., one or more cell types) will become immobilized to the surface of the biosensor by binding to the one or more specific binding substances immobilized to the surface of the biosensor. The cells in the mixed population of cells that do not bind to the specific binding substances can be optionally washed away from the surface of the biosensor or left on the surface of the biosensor. One cell type can be a class of cell types, e.g., all lymphocytes, or one particular cell type, e.g. one specific type of lymphocyte, e.g., T-cells, or one specific type of T-cell, e.g., CD8⁺ T cells.

The growth of explants taken directly from a living organism (e.g. biopsy material) is known as primary cell culture. A primary cell culture can consist of a mixed population of cell types. The time and processes needed to sort and purify primary cells from these mixed populations of cells can negatively impact the outcome of assays. In addition, the numbers of cells extracted from these methods are usually limiting, making assays that can be enabled with very few numbers of cells/well highly attractive for use with primary cultured cells. Methods are needed to determine the state, activity, and receptivity of specific subsets of primary cell populations without lengthy isolation procedures that perturb the outcome of assays in undesirable ways. Primary cells can be sorted, detected quantified and/or analyzed using the methods of the invention without deleteriously affecting the cells and the outcome of the assays. Primary cell cultures include, but not limited to, T Cells, B cells, stem cells, NK cells, monocytes, dendritic dells, endothelial cells, tumor cells, leucocytes, astrocytes, cardiomyocytes, hepatocytes; neurons. Assays that are typically run using primary cells include stimulation and functional tests such as GPCR assays, RTK assays, on channel assays, siRNA assays, viral infection assays, internal target response assays, toxicity assays, proliferation assays. Other assays test for the presence, absence, or modulation of specific cell type(s), the presence, absence, modulation of a cell surface protein(s), and further testing of the sorted cell type for response to stimulus. In one case a test might involve the purposeful mixing of cells (as cells cause changes in other cells' presence) and then sorting the purposeful mixture back into individual cell type components for further testing of the change(s) induced in the presence of the other cells. For example, a healthy cell line can be mixed with the same type of cell that is unhealthy to look for transference of disease character. Another example in a clinical setting might include the testing of patient cells for response to a pharmaceutical prior to prescription. Another clinical setting test might involve on-site real-time sorting, quantification, and testing of patient cells for cancer markers.

The one biosensor surface can be one portion on the surface of one biosensor that is contacted with the mixed population of cells (e.g., a microfluidic channel, a well, one distinct portion of a surface). Where the biosensor is incorporated into a microwell plate, each well is one biosensor surface. Each well within the microtiter plate can have different specific binding substances or different combinations of specific binding substances immobilized thereon, thereby making a panel of specific binding substances or combinations of specific binding substances that can be probed with one or more different cells and one or more different types of stimuli, incubations or test reagents.

Compounds or analytes that can stimulate cells include, e.g., hormones, growth factors, pharmaceuticals, test pharmaceuticals, differentiation factors, morphogens, cytokines, chemokines, insulin, EGF, ATP, UTP, carbanoylcholine, acetylcholine, epinephrine, muscarine, compounds that induce osmolarity changes, compounds that induce membrane depolarization, small molecule test compounds, viruses, antibodies, proteins, polypeptides, antigens, polyclonal antibodies, monoclonal antibodies, single chain antibodies (scFv), F(ab) fragments, F(ab′)₂ fragments, RNA, DNA, siRNA, Fv fragments, small organic molecules, cells, bacteria, and biological samples, e.g., blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, and prostatic fluid, and any other molecule or compound that can potentially affect a cell. Other stimuli can include, e.g., change in temperature, pH, pressure and changes in other environmental factors.

Stimuli include stimuli that “activate” or “prime” a cell. Stimuli activate or prime a cell by altering the cell's biochemical and functional activities. Cell activation can be associated with rapid induction of the expression of a number of new genes, including those encoding transcription factors, oncogenes, cytokines, early response genes, cell surface molecules, adhesion molecules, and other genes. For example, when macrophages or monocytes are activated by stimuli they can exhibit reduced motility, expression of new surface antigens, synthesis of plasminogen activator, enhanced cytotoxicity against tumor cells, increased production and release of cytokines, increased synthesis of prostaglandins/leukotrienes, increased production of reactive oxygen intermediates and other changes. Cells that have been activated can, e.g, express, down-regulate, or up-regulate production of a protein or other analyte. For example, in endothelial cells P-selectin, a cell adhesion molecule, moves from an internal cell location to the endothelial cell surface when endothelial cells are activated by, e.g., histamine or thrombin during inflammation. Different activation states of cells can be identified and classified by the phase-specific expression of novel antigens on the surfaces of activated cells, which can be determined using the methods of the invention.

Depending on the nature of the stimuli, cells can be primed only for selected functions and may not attain the full spectrum of functional capacities. Activation and priming processes can also be reversed in that some stimuli are capable of deactivating pre-activated cells, e.g., macrophage deactivation factor.

In one embodiment of the invention, specific binding substances that bind or potentially bind analytes or proteins that are expressed, up-regulated, or down-regulated when a cell is activated or primed are immobilized in the surface of a biosensor. A mixed population of cells (or purified cell population) is activated or primed (i.e., exposed to one or more stimuli) and added to the surface of a biosensor). Alternatively, the mixed population of cells can be added to the surface of the biosensor and then activated or primed. Cells that bind to the specific binding substances on the biosensor surface will become immobilized to the cell surface. Optionally, unbound cells can be washed from the surface of the biosensor. Cells can therefore be sorted, detected, quantified, and analyzed. Optionally, additional stimuli may be added to the cells and their response detected.

In another embodiment of the invention, cells can be activated or primed and then tested for inhibition of cell activation by adding stimuli that may inhibit cell activation, such as antagonists, antibodies, or drugs. For example, cells (a mixed population or purified population) can be activated or primed and then added to a biosensor surface having specific binding substances that bind or potentially bind analytes or proteins that are expressed, up-regulated, or down-regulated when a cell is activated or primed are immobilized to its surface. Optionally, the cells can be added to the surface of the biosensor and then primed or activated. One or more stimuli that may inhibit cell activation or cell priming is added to the biosensor surface and the response of the cells to the stimuli can be detected. In this manner, cells can be sorted, detected, quantified and analyzed on one biosensor surface.

The methods of the invention can be used for tissue typing, wherein the tissues, blood, or blood products of a donor and receipeint are tested prior to transplantation or transfusion. Any tissues or blood products can be subjected to tissue typing including, for example, embryos. Methods of the invention can be used to perform tissue typing by establishing the phenotype at, e.g., the HLA-A, HLA-B, HLA-C, HLA-DP, HLA-DQ, and HLA-DR loci and can be used to determine the percent reactive antibody assay. Methods of the invention can also be used in cross-matching to determine compatibility of a donated unit of blood with its intended recipient. In one example, the donor's whole blood is added to the surface of a biosensor with immobilized specific binding substances that bind white blood cells. Non-binding cells from the whole blood sample are washed away. The recipient's serum (e.g., stimuli) is added to the biosensor and a reaction is detected. If the donor's white blood cells are damaged, then a positive cross-match is the result and a transfusion is not indicated.

The sorting, enumeration, detection and analyses of cells by methods of the invention, wherein the specific binding substances are specific antibodies or ligands that bind to specific antigens or receptors on the cells have applications in, e.g., transplantation, hematology, tumor immunology and chemotherapy, genetics and sperm sorting for sex preselection, identification of cell surface-displayed protein variants with desired properties from yeast display libraries and bacterial display libraries.

Methods of the invention can be also be used to examine the volume and morphological complexity of cells, perform cell cycle analyses, examine cell kinetics such as cell proliferation, perform chromosome analysis and sorting, examine cell protein expression and localization, examine protein modifications (e.g., phospho-proteins), examine the expression of transgenic products in vivo, (e.g., green fluorescent protein or cell surface antigens such as CD markers; examine the production of intracellular antigens (e.g., cytokines, secondary mediators); examine expression of nuclear antigens; examine enzymatic activity; monitor pH, intracellular ionized calcium, magnesium, and membrane potential; examine membrane fluidity; examine apoptosis; examine cell viability; monitor electropermeabilization of cells; examine oxidative burst; characterize multidrug resistance (MDR) in cancer cells; examine glutathione production, and combinations thereof. In one example, cells are immobilized to a biosensor and are treated with compounds that stimulate G-protein coupled receptors, e.g., carbachol, which is stimulatory, and atropine, which is a competitive antagonist, effect the muscarinic acetylcholine receptor (mAChR). These effects are detectable using this invention.

In other examples, methods of the invention can be used to perform immunophenotyping, i.e., identification and quantification of cellular antigens with monoclonal antibodies. Immunophenotyping is used to diagnose and classify acute leukemias, chronic lymphoproliferative diseases, and malignant lymphomas. Treatment strategy for these diseases often depends on the diagnosis and classification of the disease. Acute leukemias are classified into two subclasses: the lymphoblastic (ALL) type and the myeloid (AML) type. ALL is further subdivided into three subtypes and ALM is further divided into eight subtypes. Many different antibodies that specifically bind cellular antigens are used for immunophenotypic analysis of hematological malignancies. Cellular antigens can include, e.g., CD1, CD2, CD3, CD4, CD5, CD6, CD7, CD8, CD10, CD11b, CD11c, CD13, CD14, CD15, CD16, CD19, CD20, CD23, CD25, CD30, CD34, CD41, CD42b, CD43, CD45, CD56, CD57, CD61, CD79a, CD103, CD117, HLA-DR, glycophorin A, TdT, and myeloperoxidase. A cell sample, e.g., a blood sample, spinal fluid, or bone marrow can be added to a biosensor surface that has immobilized antibodies that specifically bind one or more cellular antigens such that cells bearing the cellular antigens can be sorted, detected, enumerated cells and analyzed to diagnose or provide a prognosis for acute leukemias.

In some examples, a set of antibodies comprising, e.g., antibodies that specifically bind to CD19, CD20, and CD22 can be used to determine B-cell clonality, while antibodies that specifically bind to CD2, CD3, CD4 and/or CD7 can be used to T-cell clonality using mixed cell population samples. Additional antibodies would be used to diagnose a specific lymphoproliferative disorder. Antibodies specific for CD45 are useful to differentiate hematological malignancies from other neoplasms and to help detect blast cells. In another example, a weak reaction with surface immunoglobulin, a positive result with CD5, CD23, and CD43, and a negative result with CD10, CD11c, CD103, and cylin D, indicates chronic lymphocytic leukemia. In another example, multiple myeloma is caused by B cell neoplasia that results in dysregulated production and clonal expansion of malignant plasma cells that express CD138. The detection and measurement of CD138⁺ plasma cells in the bone marrow or blood can be used to diagnose and determine treatment for multiple myeloma.

Methods of the invention can also be used to diagnose minimal residual disease, with is the existence of malignant cells in a patient after remission, wherein the malignant cells are present at levels that are below the limit of detection by conventional morphological techniques. The malignant cells may cause patient relapse. Methods of the invention provide sensitive (detection limit of at least 10⁻³ cells) specific diagnosis of MRD. For example, detection of cells expressing CD10, TdT or CD34 in cerebrospinal fluid indicates MRD; and expression of TdT, cytoplasmic CD3, CD1a or CD4/CD8 in bone marrow cells indicates MRD.

Methods of the invention can be used to diagnose HIV infection to provide a prognosis by sorting, detecting, and/or enumerating cells that express CD4, CD8, and CD38 or a combination thereof. Methods of the invention can also be used to diagnose and provide prognosis information for immunodeficiency diseases, allergic disorders, and leukocyte adhesion disorders.

Methods of the invention can be used to monitor multiple drug resistance by analyzing and measuring the expression of cell surface and intracellular markers of multiple drug resistance. The efficacy of cancer chemotherapy can be monitored using the methods of the invention. Furthermore, where antibodies are used to treat cancer (e.g., antibodies specific for CD20, CD33, CD25, CD45 or CD52) methods of the invention can be used to verify binding of the antibodies and to monitor the efficacy of tumor cell eradication.

Methods of the invention can also be used for reticulocyte enumeration, reticulocyte maturation index determination, immature reticulocyte fraction determination, platelet function analysis, platelet surface receptor quantitation and distribution analysis, platelet-associated IgG quantitation assays, reticulated platelet assays, fibrinogen receptor occupancy studies, detection of activated platelet surface markers, cytoplasmic calcium ion measurements, platelet microparticle assays, cell function analysis, tyrosine phosphorylation assays using antiphosphotyrosine antibodies, calcium flux analysis using Ca2+ indicators, oxidative metabolism assays, and cellular proliferation assays.

Methods of the invention can also be used to sort, detect, quantify, and analyze bacteria, fungi, parasites and viruses in biological, environmental or food samples. If the microorganisms are intracellular, the cells can be permeabilized or lysed. Advantageously, the microorganisms do not need to be cultivatable.

### Methods of Screening Two or More Cell Types on a Single Biosensor Surface

Prior to the instant invention, most cell-based assays allowed screening of a single target in a single cell line, or multiple targets or parameters in a single cell line (high content screening). Technology for assaying multiple cell lines by tagging individual cell lines simultaneously has been described by Besko et al. (Journal of Biomolecular Screening, Vol. 9, No. 3, 173-185 (2004)). This technology, however, requires detectably labeling each individual cell line so that they can be distinguished from each other.

One barrier to the adoption of label-free cell-based assays by high throughput screening groups is cost. If multiple targets can be screened simultaneously, the per well cost for screening is divided by the number of targets being screened. For instance, if three targets are screened simultaneously, the per well cost of screening is one third of what it would be if only one target at a time was screened. In addition, high throughput screening with primary cultured cells is highly desirable yet the cost of isolating or purchasing primary cultured preparations can be prohibitive. If fewer primary cultured cells/well can be utilized in screening assays that still enable robust assay readouts, the per well cost of screening with these limiting cell types will be decreased.

The instant invention provides methods for screening, assaying, and quantifying multiple cell lines simultaneously in a completely label-free manner on a single biosensor surface, such as a single well of a microplate. There is no need to detectably label each cell line for identification following the screening/assaying activity.

Screening multiple cell lines with some detection devices is limited by the amount of signal dilution that can be tolerated from adding multiple cells lines. For instance, assaying two cell lines in the same well using certain detection devices will give half the signal as assaying one single cell line. Also, screening multiple cell lines with some detection devices is confounded by the assay readout which does not distinguish responses from individual cell types but rather provides a readout consisting of an average of the signals from all of the individual cell types combined. This problem of signal dilution is circumvented by using the BIND® SCANNER to detect signals (however, screening multiple cell lines in one vessel can also be detected using the BIND® READER, see FIGS. 28-30). Because individual cells responding to stimulus can be identified and counted, more cell lines can be simultaneously assayed without concern for signal dilution and the responses of individual cell subpopulations can be measured.

One functional advantage of the invention is that by screening multiple cells against the same test reagent in a single well, the assay has a built-in test of test reagent specificity. If the same test reagent is found to inhibit the activation of multiple cell lines expressing different receptors, then the test reagent is likely promiscuous or cytotoxic. Currently, this can be done by screening one test reagent in multiple wells containing different cells. The instant invention allows the user to screen one test reagent against mixed cell populations (such as cardiomyocytes and hepatocytes) in a single well. The per well cost of screening is effectively divided by the number of targets being screened simultaneously.

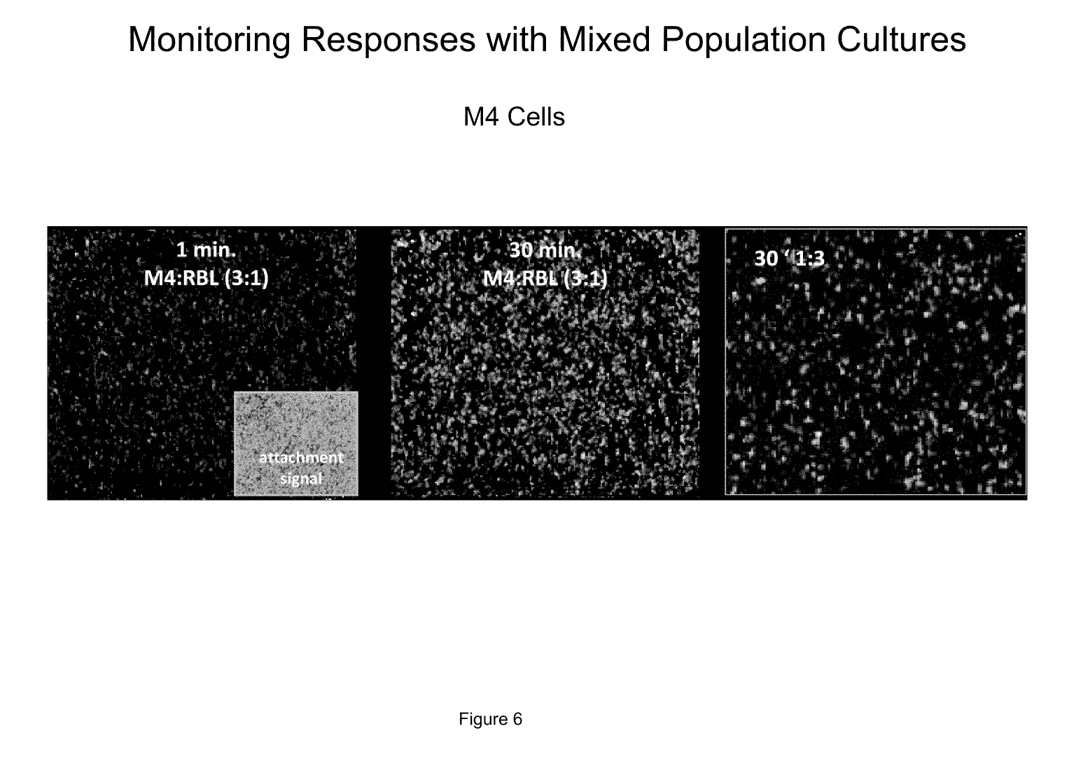

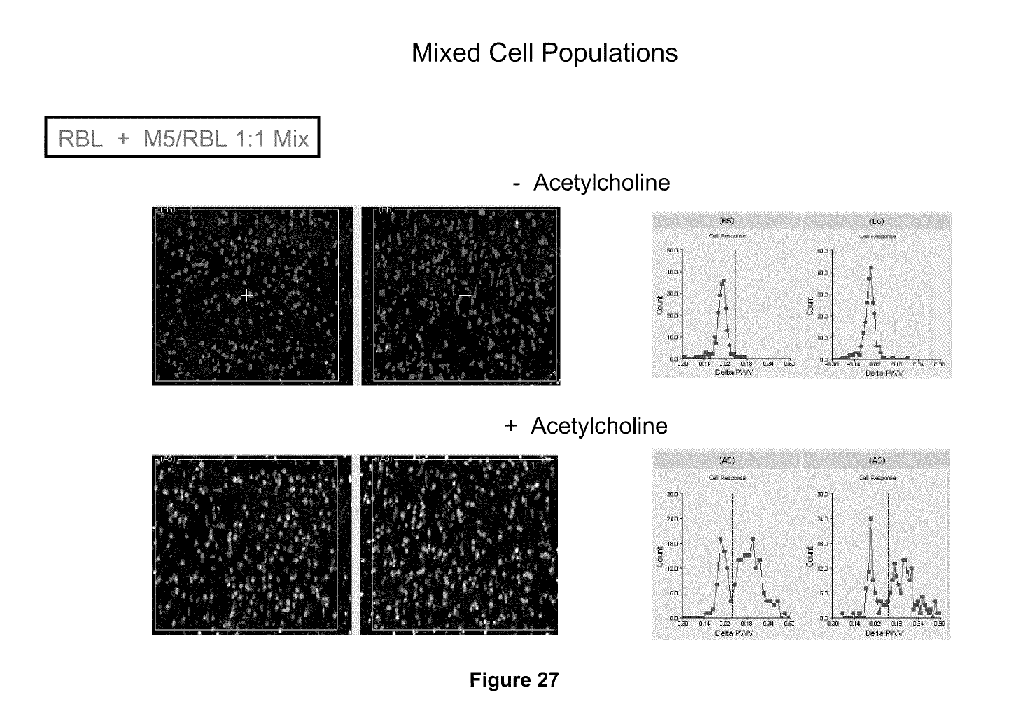

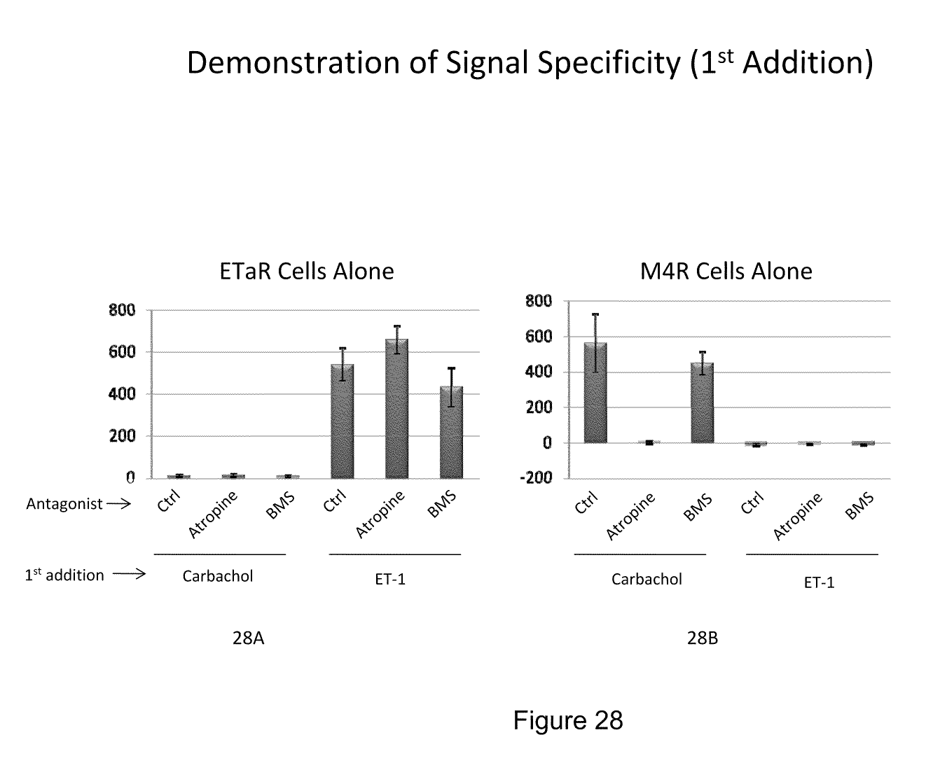

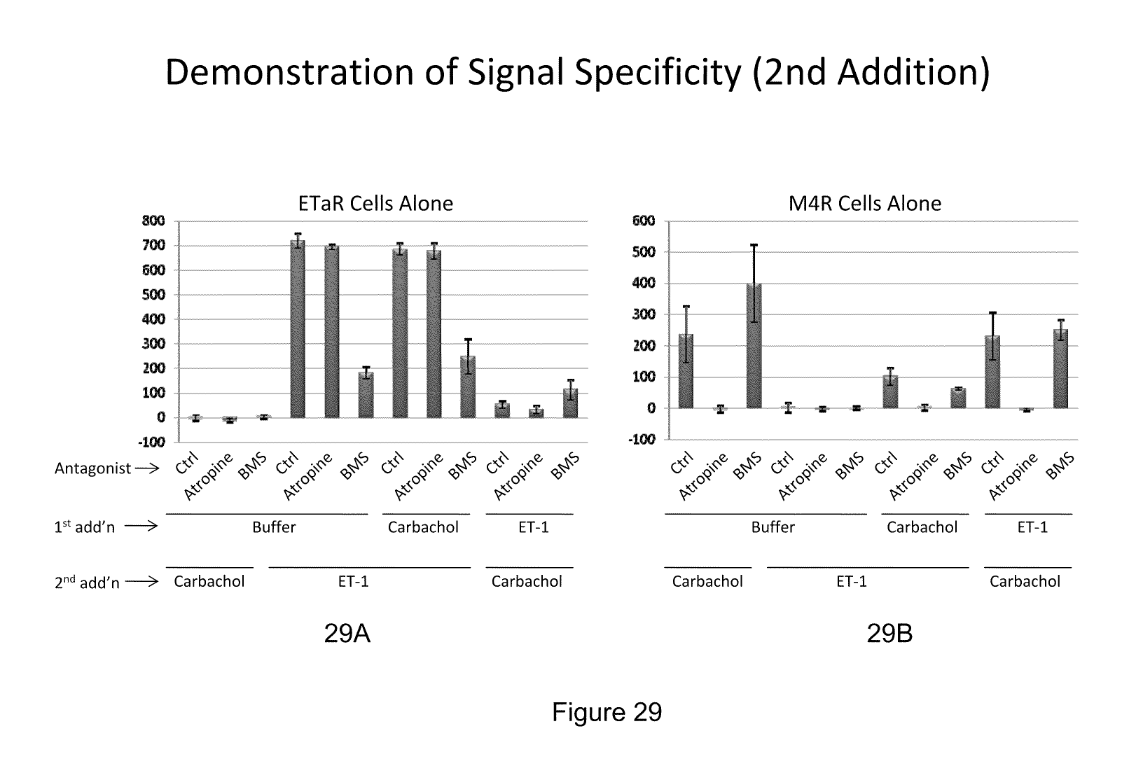

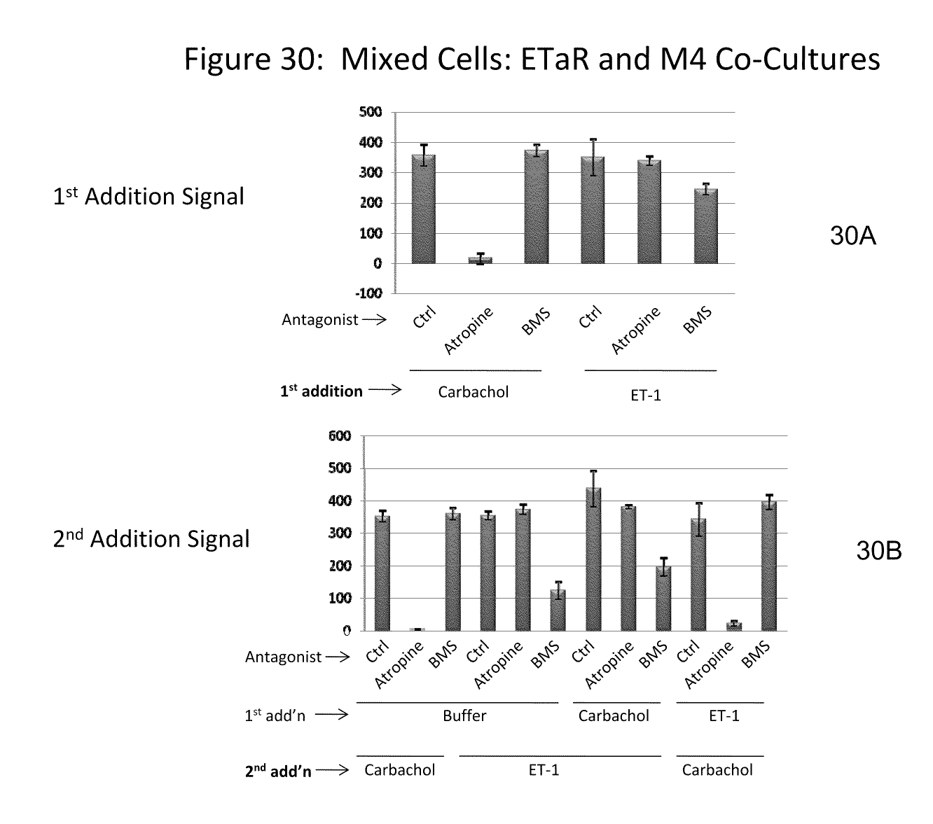

The response of 2, 3, 4, 5, 6, 7, 8, 9, 10, 20, 30, 40, 50, 100 or more types of cells in one vessel to stimuli, a test reagent, or an incubation step can be detected using methods of the invention wherein the cells do not comprise detectable labels. The methods comprise applying the two or more types of cells to a vessel, wherein an internal surface of the vessel comprises a colorimetric resonant reflectance biosensor surface or a grating-based waveguide biosensor surface, wherein the biosensor surface has one or more specific binding substances or ligands immobilized to its surface and wherein the one or more specific binding substances or ligands can bind one or more of the two or more types of cells. Cells that do not bind to the specific binding substances or ligands can optionally be washed away although a wash step is not necessary. The two or more cells types can be exposed to stimuli, a test reagent, or an incubation step. The response of the two or more cell types to the stimuli or test reagent can be detected by a BIND® SCANNER (high resolution colorimetric resonant reflectance biosensor system), see, e.g. FIGS. 6 and 27. The response of each cell type to the test reagent, stimuli or incubation can be individually detected and analyzed by examining the signal from each individual cell on the biosensor surface.

The response of the two or more cell types to the stimuli or test reagent can be also detected by a BIND® READER (colorimetric resonant reflectance biosensor system). For example, Endothelin receptor expressing cells (ETaR) and M4 muscarinic receptor expressing cells (M4R) were plated on CA2 cellular matrix-coated colorimetric resonant reflectance biosensor plates in starvation media. The cells were pre-treated with antagonists (either atropine to inhibit M4 or BMS to inhibit ETaR) for 30 min. The cells were then treated with either 10 uM carbachol or 50 nM ET-1. Endpoint responses detected on a BIND™ Reader are shown in FIG. 28. FIG. 28A shows ETaR cells plated alone. FIG. 28B shows M4R cells plated alone. Data are referenced to buffer controls. Mean+/−SD of four replicates shown. ETaR cells respond to ET-1, but not to carbachol, showing specificity of the response. The concentration of BMS used was not high enough to completely inhibit the ET-1 response. M4R cells respond to carbachol, but not to ET-1, showing specificity of the response. Atropine completely inhibited the carbachol response.

The ETaR cells and M4R cells were then treated with a second ligand. For instance, ETaR cells that were treated with carbachol previously were now treated with ET-1. Endpoint responses were detected on a BIND™ Reader are shown in FIG. 29. FIG. 29A shows ETaR cells plated alone. FIG. 29B shows M4R cells plated alone.

The results show that ETaR cells respond to ET-1, even after carbachol stimulation and that M4R cells respond to carbachol, even after ET-1 stimulation. BMS was more effective at blocking ET-1 signal with longer incubation time. There is some carbachol signal from the first addition showing through in the M4 cells upon ET-1 stimulation. Likewise, there is ET-1 signal from the first addition showing through in the ET-1 cells after carbachol stimulation. Therefore, it is advantageous to allow previous signal to completely saturate before the second addition.

FIG. 30 shows both ETaR cells and M4R cells cultured in the same wells with various additions of atropine, BMS, carbachol and ET-1 as indicated in FIGS. 30A-B. The Figures demonstrate that two types of cells can be plated in the same well, and that individual activation of each cell type can be separately detected. Therefore, complex mixtures of cells from, for example, native tissue can be differentiated by, for example, ligand response or receptor expression. The presence or absence of specific cell types in the mixture of cells can be therefore be determined.

### Differential Response to Ligands, Stimuli or Incubation

Each type of cell in a mixed population of cells can have a different response and therefore PWV reading to a stimulus, test reagent or incubation step. Distinct cell types can display PWV signals on the biosensor that are distinct from each other based on the PWV signal averaged across the pixels that define the cells' response to a stimuli, test reagent, or incubation step. That is, one cell type on the biosensor can react strongly to the stimuli, test reagent or incubation step and display a higher PWV than a second cell type on the biosensor that reacts weakly to the stimuli, test reagent or incubation step and display a lower PWV. A BIND® SCANNER or BIND® READER acquisition is performed to obtain PWV images of the biosensor surface. The initial cell attachment images are analyzed to find individual cells, make morphological measurements on each cell, and classify cells into two or more sub-populations. The cell attachment images are processed to remove local background variation and sharpen edges. Images are “thresholded” to identify PWV values that are sufficiently above background. Contiguous collections of suprathreshold pixels are labeled as individual cells. For each cell that is segmented from the cell attachment image, morphological metrics are computed. For assays where the cell types in a mixed population can be categorized based upon cell size, the area of each cell is determined. For assays where cell types can be differentiated based on shape-based characteristics, metrics such as circularity are provided. Using one or more morphological metrics relevant for the assay, cells are classified into sub-populations wherein one population exhibits the desired morphological characteristics. For each well, a binary image (“mask”) that labels cells from the designated morphological sub-population is carried forward in the data analysis workflow. This mask is applied to images from a subsequent acquisition where a test reagent, stimuli or incubation is added to/performed on the mixed cell population; the mask allows the cell response to the test reagent, stimuli or incubation to be quantified from only those cells in the morphological sub-population.

### Differential Response to Secondary Ligand or Stimuli

Cells of interest within a mixed population can be also be differentiated based upon their response to a secondary ligand. Distinct cell populations in a vessel can respond differentially to test reagents or stimuli yielding PWV shifts that can be used as signatures to identify these subpopulations. For example, one might be interested in measuring the response of neurons in a primary cultured preparation to capsaicin, a pain stimulus. In the cell preparation multiple cell types (neurons, oligodendrocytes, astrocytes) might be present that all respond to capsaicin, yet the interest is in measuring the responses in neuronal cells. Of the cells in the vessel, only neurons will respond (PWV shift) to nerve growth factor (NGF). Thus, the delta PWV response to capsaicin can be measured for all of the cells in the vessel, followed with a second stimulation with NGF to determine which of the cells in the vessel are neurons.

To measure the response of a mixed population of cells to a primary ligand or test reagent in combination with a secondary known ligand or test reagent a BIND® SCANNER acquisition is performed to obtain PWV images of the microplate wells in which cells from the culture have been attached to the biosensor surface. These cell attachment images are analyzed to segment all individual cells. A BIND® SCANNER acquisition is performed following addition of a library of ligands, one per well in a biosensor microplate. It is unclear at this stage whether the sub-population of cells of interest has responded to the primary ligand. A secondary ligand is then administered that is known to stimulate with specificity the cell type of interest, and the final BIND® SCANNER acquisition is performed and analyzed. The cell attachment images are processed to remove local background variation and sharpen edges. Images are “thresholded” to identify PWV values that are sufficiently above background. Contiguous collections of suprathreshold pixels are labeled as individual cells. Using the cell definition mask segmented from the cell attachment image, the cells in the BIND® SCANNER images obtained after addition of the secondary ligand are processed. For each cell, its PWV response to the secondary ligand is calculated. Cells are classified into sub-populations. The sub-population of cells that have the largest responses to the secondary ligand is retained. For each well, the binary mask identifies the sub-population of cells that has been differentiated based upon the secondary ligand is then applied to the data from the primary ligand. The mask from the secondary ligand addition allows the cell response to the primary ligand to be quantified from only those cells in the sub-population of interest.

### Differential Response of Cells Based on Attachment Signal

Cells can be differentiated based on their attachment signal. When cells attach or bind to the surface of the biosensor they display an attachment signal, that is, an increase in PWV at the pixels where the cells attach. Distinct cell types can display PWV attachment signals on the biosensor that are distinct from each other based on the strength of the signal averaged across the pixels that define the cell attachment signal. That is, one cell type on the biosensor can bind strongly to the biosensor and display a higher PWV and consequently higher cell attachment signal than a second cell type on the biosensor that binds weakly to the biosensor and display a lower PWV and consequently lower cell attachment signal. A BIND® SCANNER acquisition is performed to obtain PWV images of the biosensor surface to which cells from the culture have attached. These cell attachment images are analyzed to find individual cells, determine the strength of the attachment signal of each cell, and classify cells into two or more sub-populations, as described below. The cell attachment images are processed to remove local background variation and sharpen edges. Images are “thresholded” to identify PWV values that are sufficiently above background. Contiguous collections of suprathreshold pixels are labeled as individual cells. For each cell that is segmented from the cell attachment image, its mean PWV value is calculated. The PWV value is proportionate to the strength of the cell's attachment (the amount of mass from the cell bound to the biosensor surface). Cells are classified into sub-populations wherein one population exhibits the cell attachment signal of interest. For each well, a binary image (“mask”) that labels cells from the designated cell attachment sub-population is carried forward in the data analysis workflow. This mask is applied to images from a subsequent acquisition where a test reagent or stimulus is added to the mixed cell population in a well; the mask allows the cell response to the test reagent or stimulus to be quantified from only those cells in the well that are in the cell attachment sub-population.

Distinct cell types can display PWV attachment signals on biosensors that are distinct from each other based on the surface area of cell attachment signals as defined by contiguous pixels exceeding a predefined PWV threshold. That is, one cell type on the biosensor can bind to the biosensor such that each cell covers an average biosensor surface area that is significantly larger or smaller than a second cell type on the biosensor. Distinct cell types can also display PWV attachment signals on biosensors that are distinct from each other based on the overall shape of the cell attachment signals as defined by contiguous pixels exceeding a predefined PWV threshold. For example, two cell types that attach to optical biosensors yielding attachment signals of similar surface area might still be further distinguished from each other based on a pyramidal versus oblong cell morphology. A BIND® SCANNER acquisition is performed to obtain PWV images of the biosensor surface. These cell attachment images are analyzed to find individual cells, make morphological measurements on each cell, and classify cells into two or more sub-populations. The cell attachment images are processed to remove local background variation and sharpen edges. Images are “thresholded” to identify PWV values that are sufficiently above background. Contiguous collections of suprathreshold pixels are labeled as individual cells. For each cell that is segmented from the cell attachment image, morphological metrics are computed. For assays where the cell types in a mixed population can be categorized based upon cell size, the area of each cell is determined. For assays where cell types can be differentiated based on shape-based characteristics, metrics such as circularity are provided. Using one or more morphological metrics relevant for the assay, cells are classified into sub-populations wherein one population exhibits the desired morphological characteristics. For each well, a binary image (“mask”) that labels cells from the designated morphological sub-population is carried forward in the data analysis workflow. This mask is applied to images from a subsequent acquisition where a test reagent or stimuli is added to the mixed cell population; the mask allows the cell response to the test reagent or stimuli to be quantified from only those cells in the morphological sub-population.

Distinct cell types can display PWV attachment signals on biosensors that are distinct from each other based on their reaction over time as they attach to the sensor surface. For example, a first cell type can be defined by the population of cells in a heterogeneous mix that attaches to the biosensors rapidly (e.g. within the first 20 minutes following cell addition), whereas a second cell type can display a slower attachment signal (e.g. saturating closer to an hour after cell addition). Therefore, cells of interest within a mixed population can be differentiated based upon their response over time. A BIND® SCANNER acquisition is performed to obtain PWV images of the biosensor where cells from the culture have been attached to the biosensor surface. These cell attachment images are analyzed to segment all individual cells. After test reagents or stimuli are added to the biosensor, a BIND® SCANNER acquisition is performed in which the microplate is read repeatedly for some duration. The cell attachment images are processed to remove local background variation and sharpen edges. Images are “thresholded” to identify PWV values that are sufficiently above background. Contiguous collections of suprathreshold pixels are labeled as individual cells. Using the cell definition mask segmented from the cell attachment image, the cells in the BIND® SCANNER images obtained after addition of the ligand are processed. For each cell type, its PWV response is measured in each of the BIND® SCANNER time course images to generate a time course profile for the cell. Metrics that characterize each time course are generated, such as the time to maximal response and the range (maximum−minimum) over which the response changes. Cells are classified into sub-populations wherein one population exhibits the time course profile of interest. For each well, a binary image (“mask”) that labels cells from the designated time course profile sub-population is used to quantify the cell response to the test reagent or stimuli from only those cells in the well that are in the designated sub-population.

### Differential Response Kinetics Over Time

Distinct cell populations in a vessel can display delta PWV responses to a particular stimulus that are distinguishable from other cells based on the kinetics of the response over time. For example, a neuronal response to capsaicin might be characterized by a rapid positive PWV shift that plateaus whereas the astrocyte and oligodendrocyte responses to the same stimulus may be characterized by a transient positive PWV shift that rapidly returns to baseline. Any change in response kinetics over time can be used differentiate between different cell types on a biosensor surface or to identify a cell type on the cell surface when the response of a cell type to a test reagent, stimuli or incubation is known.

Therefore, the invention provides methods for detecting differential responses of two or more types of cells in one vessel to stimuli or a test reagent, wherein the two or more types of cells do not comprise detectable labels. The methods comprise applying the two or more types of cells to the one vessel, wherein the vessel comprises a colorimetric resonant reflectance biosensor surface, a grating-based waveguide biosensor surface, or a dielectric film stack biosensor surface, wherein the biosensor surface has one or more specific binding substances immobilized to its surface and wherein the one or more specific binding substances can bind one or more of the two or more types of cells. The two or more types of cells are allowed to bind to the one or more specific binding substances and the differential responses of the two or more cell types are detected. The differential responses can be, for example, different times of the two or more types of cells to attach to the one or more specific binding substances, different cell attachment morphologies displayed by the two or more types of cells to the one or more specific binding substances, and different strengths of attachment of the two or more cell types to the one or more specific binding substances. The method can further comprise exposing the two or more cell types to one or more test reagents or stimuli and detecting the differential responses of the two or more cell types. The differential responses can be different strengths of response of the two or more cell types to the one or more test reagents or stimuli, different cell morphologies displayed by the two or more types of cells in response to one or more test reagents or stimuli, different cell responses of the two or more cell types to the one or more test reagents or stimuli over time, or different response kinetics of the two or more cell types over time.

The method can further comprise exposing the two or more cell types to a first test reagent or first stimuli and detecting the responses of the two or more cell types to the first test reagent or first stimuli. The two or more cell types are then exposed to a second test reagent or second stimuli, wherein the response of one of the cell types in the two or more cell types to the second test reagent or second stimuli is known. Alternatively, the response of the one of the cell types to the first test reagent is known and the response to the second test reagent is unknown. The responses of the two or more cell types to the second test reagent or second stimuli are detected. The one of the cell types in the two or more cell types that have a known response to the second test reagent or second stimuli are identified and the differential response of the two or more types of cells are detected. The one or more test reagents or stimuli can be expressed by one or more cells of the two or more types of cells present on the biosensor surface.

The invention also provides methods of detecting the presence of a first cell type in a mixed population of cells, wherein none of the cells in the mixed population of cells comprise detectable labels. The methods comprise applying the mixed population of cells to one vessel, wherein the vessel comprises a colorimetric resonant reflectance biosensor surface, a grating-based waveguide biosensor surface, or a dielectric film stack biosensor surface, wherein the biosensor surface has one or more specific binding substances immobilized to its surface. The mixed population of cells is allowed to bind to the one or more specific binding substances, wherein the first cell type has a differential response from the other cells of the mixed population of cells to binding to the one or more specific binding substances. Differential responses of the mixed population of cells are detected, wherein the presence of the first type of cells is detected by their differential response. The percentage of the first type of cells in the mixed population of cells can be determined.

The invention also provides a method of detecting the presence of a first cell type in a mixed population of cells, wherein none of the cells in the mixed population of cells comprise detectable labels. The method comprises applying the mixed population of cells to one vessel, wherein the vessel comprises a colorimetric resonant reflectance biosensor surface, a grating-based waveguide biosensor surface, or a dielectric film stack biosensor surface, wherein the biosensor surface has one or more specific binding substances immobilized to its surface. The mixed population of cells is allowed to bind to the one or more specific binding substances. The mixed population of cells is exposed to one or more test regents or stimuli, wherein the first cell type has a differential response to the one or more test reagents or stimuli as compared to the other cells in the mixed population of cells. The differential response of the first cell type to the one or more test reagents or stimuli is detected. If the differential response is detected, then the first cell type is present in the mixture of cells. The percentage of the first type of cells in the mixed population of cells can be determined. The one or more test reagents or stimuli can be expressed by one or more cells of the mixed population of cells present on the biosensor surface.

These methods can be useful in many real world applications. For example, assaying complex mixtures of cells from native tissue or any mixed cell population can be completed with the methods of the invention. An individual type of cell population within a mixed population can be differentiated by their response to ligand, cytotoxic agent, or any other stimulus, then the cell type or target type presence or absence in the mixture can be determined. These methods can be used, for example, to identify cancer cells by their response to stimulation, when healthy tissue does not respond; to identifying cancer stem cells in a tumor by their response to stimulation when non-stem cells do not respond; to detect the presence of specific circulating cell types in blood and/or serum samples; and to determine the presence or absence of specific cell biomarkers or cell proteins.

Methods of the invention allow for quantification of the amount of each cell type within a mixed population of cells. These methods can be used to, for example, identify the percentage of cancer cells in a mixed population by their response to stimulation, when healthy tissue does not respond; identify the percentage of cancer stem cells in a tumor by their response to stimulation when non-stem cells do not respond; identify what percentage of a stem cell population has differentiated into intermediate progenitor cells; identify what population of terminally differentiated cells have de-differentiated back into stem cell-like populations such as induced pluripotent stem cell populations; detect the presence of specific circulating cell types in blood and/or serum samples; determine the purity of an isolated cell population; and determine the percentage presence or absence of specific cell biomarkers or cell proteins.

Methods of the invention can be used to determine interactions between cells. The treatment of a mixture of cells producing materials that affect neighboring cell types can be exposed to compounds that, for example, abrogate the production activity or compounds that check that the response to the produced material is disrupted. For example, these methods could be used in later stage pre-clinical trials where in vivo like cell systems are required for complex analyses of a test drug compound effect. For example, human cortical neurons are encouraged to form network structures or axonal bundles in the presence of certain cell types such as Schwann cells. This encouragement is owing primarily to materials that the Schwann cells make and put into the environment around them. Additionally, cancer metastasis that is encouraged by chemicals produced by neighboring cells can be detected.

Methods of the invention can be used to determine the presence or absence of a given cell type within a mixed population, but additionally, one could determine the selectivity or sensitivity to external stimuli of each cell type in a mixed population if the different cell types within the population are known or can be distinguished. Potential applications include identifying agents that selectively kill or otherwise affect a fraction of the population, including but not limited to unwanted cells (cancerous, infected, etc.), specific cells, cells in a population containing healthy, normal, activated, transformed, or unhealthy cells.

Methods of the invention can be used to perform highly parallel testing of sample reagents and cell lines, for example testing multiple antagonists/agonists simultaneously against multiple cell lines. Multiple antagonists can be tested in parallel by adding mixtures of antagonists to biosensor wells. Any wells showing positive hits can be deconvoluted in a second step i.e. by testing individual cell lines against individual compounds from the mixture. Similarly, multiple agonists could be tested to discover new agonists to a given receptor or to deorphan an orphan receptor.

### Analysis of Stem Cell and Other Cells

One mode of cell analysis, including stem cell analysis, incorporates label-free detection utilizing the BIND® READER or BIND® SCANNER together with BIND microplate biosensors. In this method, the microplate biosensors are coated with extracellular matrix material or other specific binding substances and subsequently incubated with stem cells. The stem cells adhere to the extracellular matrix or specific binding substances and test compounds or stimuli are added. Morphological and adhesion changes are monitored using the BIND® READER or BIND® SCANNER. In some cases it may be preferable to use the BIND® SCANNER, a high-resolution label-free detection instrument capable of single cell analysis. Stem cell populations, by their nature, are not homogeneous populations of cells. Furthermore, they may not differentiate homogenously. Therefore, the BIND® SCANNER can measure and distinguish these mixed populations of cells.

Cells, such as stem cells, can attach to the biosensor of the invention and spread out. The attachment of the cells to the biosensor can be monitored in real time. The methods of the invention can be used to detect morphological changes in single cells or populations of cells. For example, scanning electron micrographs demonstrate of the effect of ATP on HEK cells expressing a rat P2X7 receptor. Control cells show typical morphology of HEK cells with a rough surface and both fine filopodia and sheet-like lamellipodia, while cells exposed to ATP for 2 min show a smooth surface and numerous large (1 m) blebs and small (0.5 m) microvesicles. The methods of the invention can detect these and other morphological changes without the use of labels or micrography.

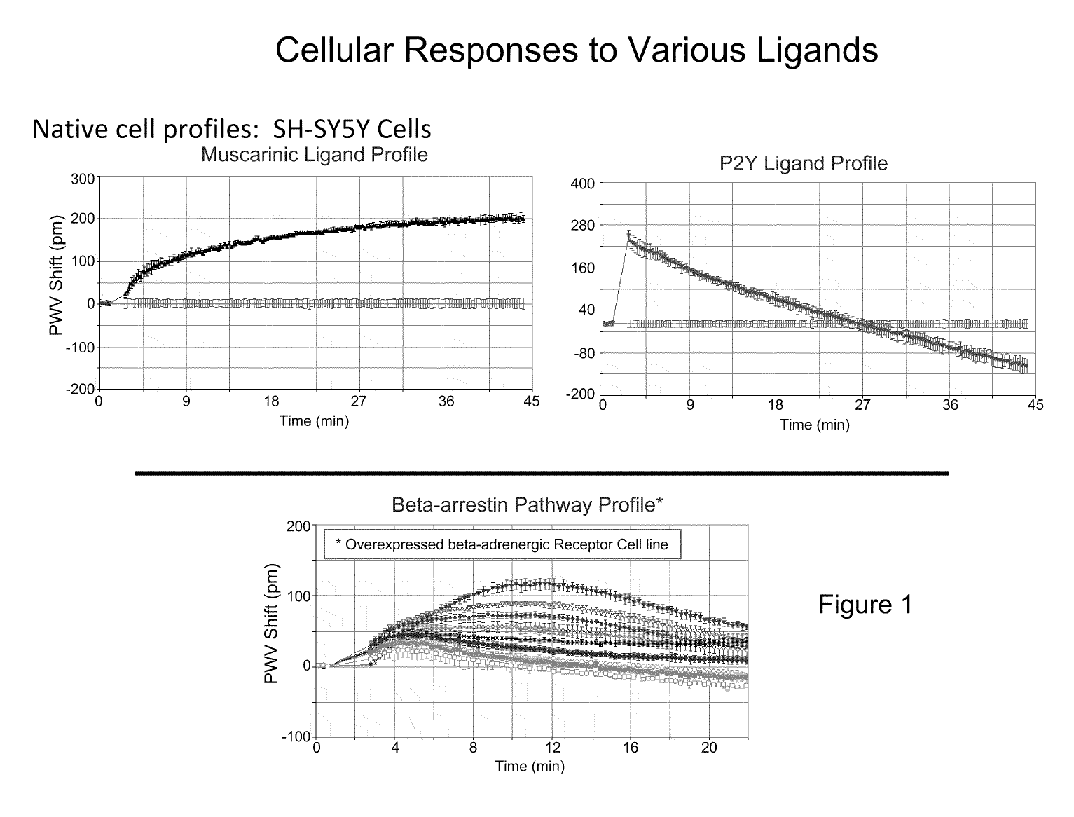

Cells each have a signature response to a ligand that is added to the surface of a biosensor to which the cells are attached or resting on. FIG. 1 shows the signature response for SH-SY5Y cells to muscarinic, P2Y, and beta-arrestin ligands on a colorimetric resonant reflectance biosensor microwell plate. Since each type of cell has a signature response for each type of ligand, a mixed population of cells can be assayed together. For example, different types of cells or cells at different stages of differentiation (or combinations thereof) can be added to a surface of a biosensor of the invention (e.g., a microtiter well). A ligand can be added to the biosensor surface and the reaction of the cells to the ligand is detected. The presence or absence of certain cells can be determined based on the cells' response to the ligands. Additionally, the proportion of reacting/non-reacting cells in the population can be determined. That is, if a population of cells contains two or more types of cells (e.g., cancerous cells that react to a ligand and non-cancerous cells that do not react to a ligand), the proportion of cancerous cells to non-cancerous cells can be determined by determining the reaction of each of the cells in the well to the ligand.

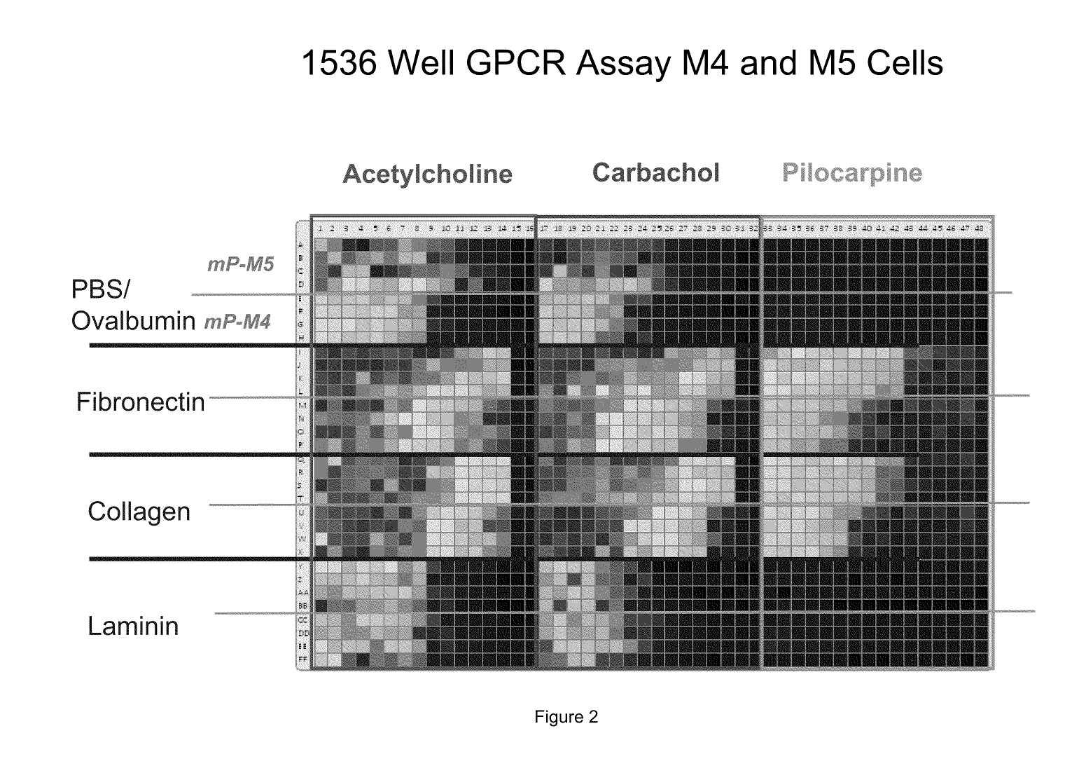

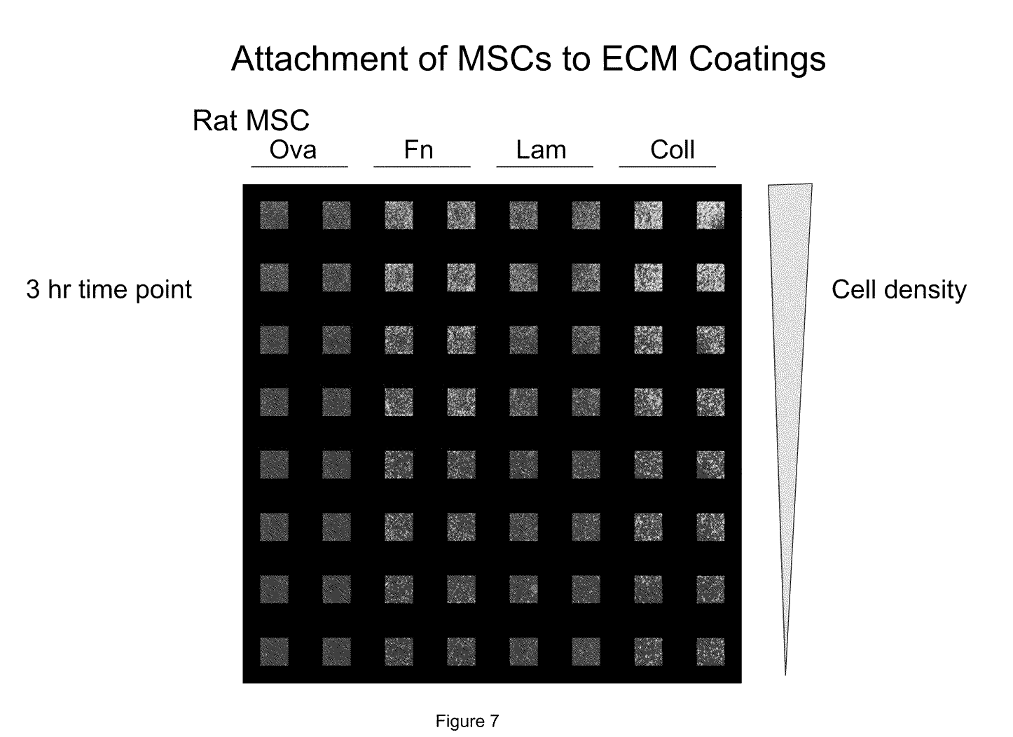

In certain cases cells, such as stem cell or primary cells, have varying reactions to ligands depending upon what extracellular matrix component is present on the surface of the biosensor. This preference can be determined for each type of cell. FIG. 2 shows the reaction of mP-M5 and mP-M4 cells to 3 ligands: acetylcholine, carbachol, and pilocarpine when the cells are on colorimetric resonant reflectance biosensors comprising PBS/ovalbumin, fibronectin, collagen or laminin. The mP-M5 and mP-M4 cells show the best reaction to the ligands when they are on biosensors comprising fibronectin or collagen. FIG. 7 shows the rat MSC cell attachment to colorimetric resonant reflectance biosensors comprising either ovalbumin, fibronectin, laminin or collegen. MSC cells attach to biosensors comprising collagen better than the other surfaces. Cells can be tested to determine the best ligand/ECM coating for attachment to the biosensor.

In stem cell research, populations of less than 1,000 cells are often used in assays. Cell populations of less than 1,000 cells can be readily assayed using the methods of the invention. Methods of the invention can be used to assay less than about 1,000, 750, 500, 100, 50, 10 or 5 cells on a single biosensor surface such as a microfluidic channel or microtiter well. Furthermore, a single cell can be assayed using the methods of the invention.

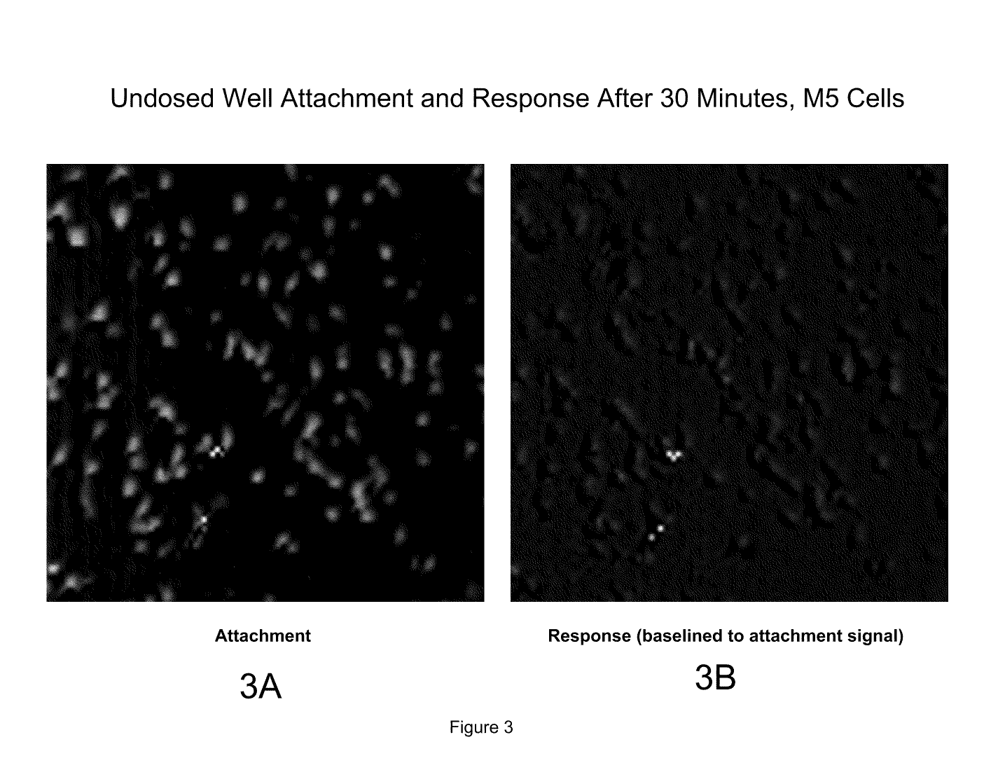

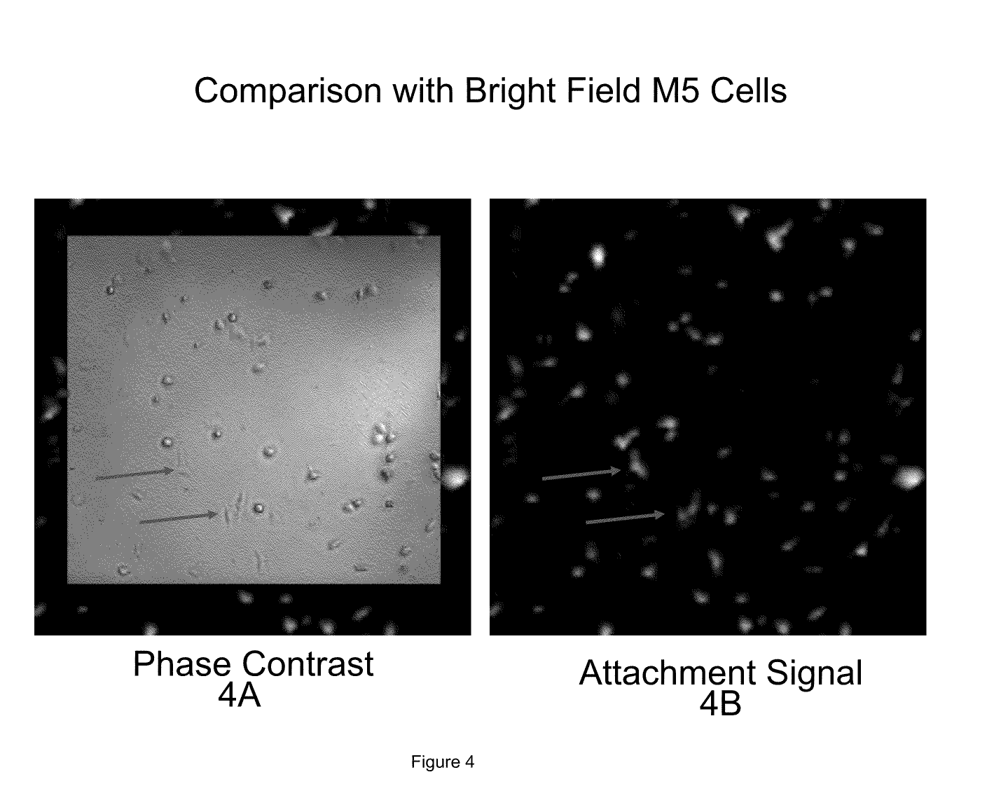

FIG. 3A shows the signal generated by M5 cells attaching to a colorimetric resonant reflectance biosensor. The BIND® SCANNER identifies cell location based on attachment signal and the response to stimuli is measured only where the cells are located. The empty space is not factored into the response measurement resulting in greater sensitivity. Robust dose-response profiles down to about 100-150 cells in a 384 well dish can be obtained. FIG. 3B shows a scan that was completed 30 minutes after the cells attached to the biosensor. The signal from the cell attachment has been zeroed out. Therefore, after attachment, the cells have demonstrated no other change in morphology. FIG. 4A shows a phase contrast image of cells from the top side of the cells (side opposite of the cell attachment to the colorimetric resonant reflectance biosensor), while the FIG. 4B shows the attachment signal of the same cells from the bottom side of the cells (the side of the cell that is bound to the biosensor).

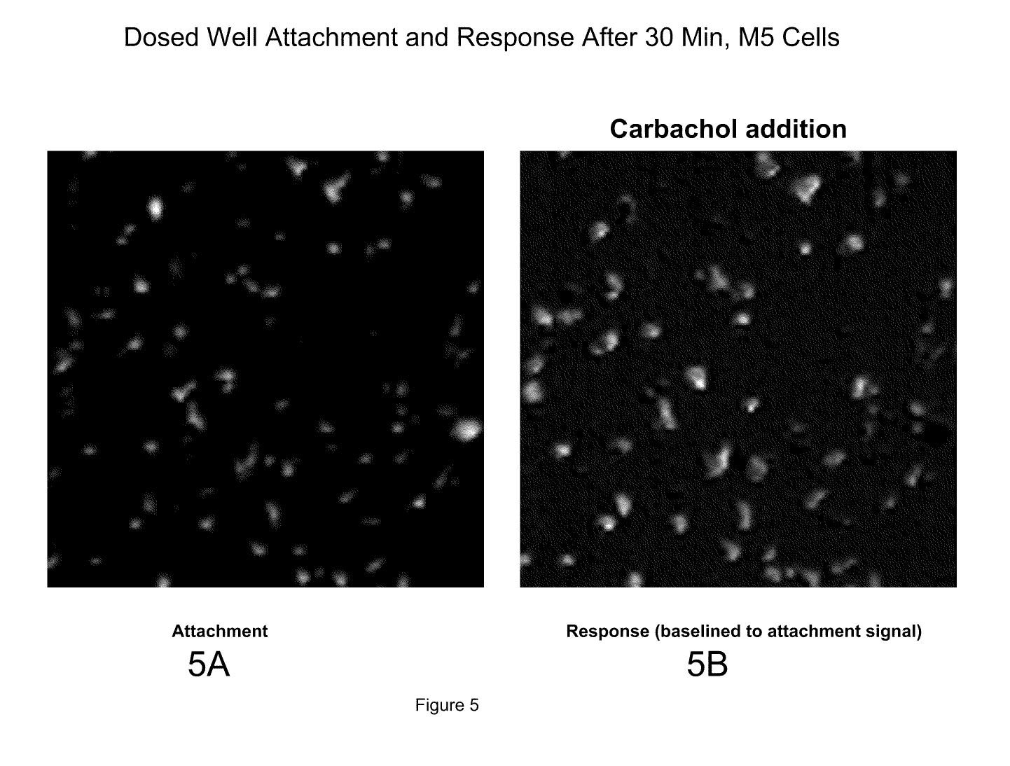

FIG. 5A shows the attachment response of M5 cells to a colorimetric resonant reflectance biosensor. FIG. 5B shows the response of the M5 cells to the addition of carbachol. The signal has been baselined to the attachment signal. Therefore, all of the response is due to the addition of carbachol, and not due to the attachment reaction. Where no carbachol is added no cell response is detected. FIG. 5, right panel, demonstrates that the signal generated by each cell is not uniform. That is, more signal is seen around the edges of the cells where the cells are moving or changing morphology in response to the carbachol.

FIG. 6 shows a mixed population of M4 cells and RBL parental cells that were added to a colorimetric resonant reflectance biosensor. M4 cells have more receptors for carbachol than the RBL cells. 10 μM of carbachol was then added to the cells. The middle panel shows a 3:1 ratio of M4 cells to RBL cells 30 minutes after the carbachol is added to the cells. The right panel shows a 1:3 ratio of M4 cells to RBL cells 30 minutes after the carbachol is added. The middle panel of FIG. 6 shows more signal than the right panel because more M4 cells are present than RBL cells, each M4 cell having more receptors for carbachol.

RBL and M5/RBL cells were mixed in a 1:1 ratio and were plated in colorimetric resonant reflectance biosensor wells. The cells were allowed to attach to the biosensor and the attachment reaction was detected on a BIND® SCANNER. The results are shown in FIG. 27A and FIG. 27B. Acetylcholine was added to the biosensor surface. Only M5/RBL cells react to acetylcholine. The reaction of the cells to the acetylcholine is shown in FIG. 27C and FIG. 27D. Approximately 50% of a 1:1 mixed population of RBL+M5/RBL cells responded to acetylcholine. The responding cells can be gated and analyzed for quantitative responses (e.g., responses to additional test reagents or stimuli) independent of non-responding population. Therefore, the presence of different types of cells on a biosensor can be detected when their response to a ligand is known.

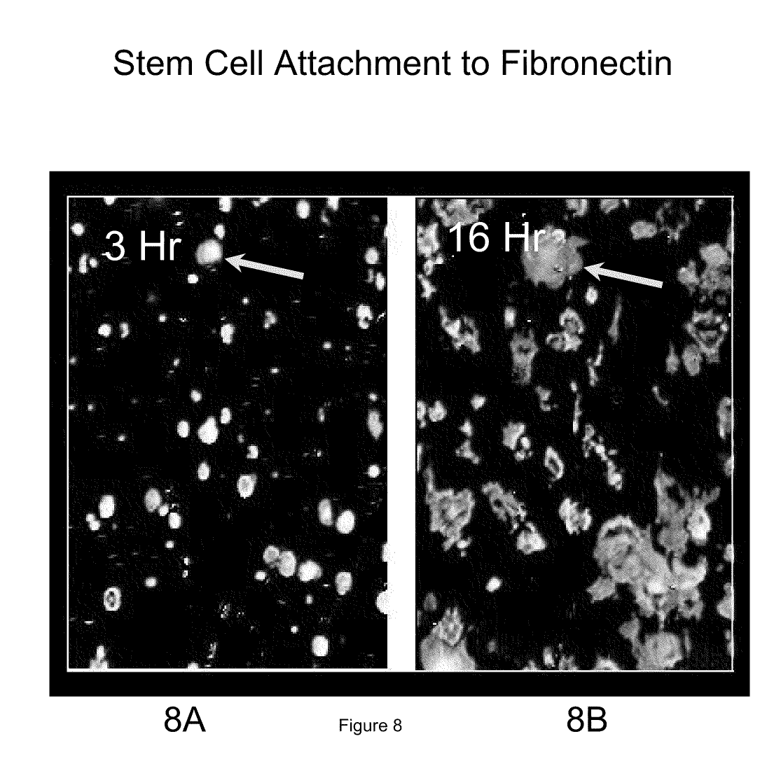

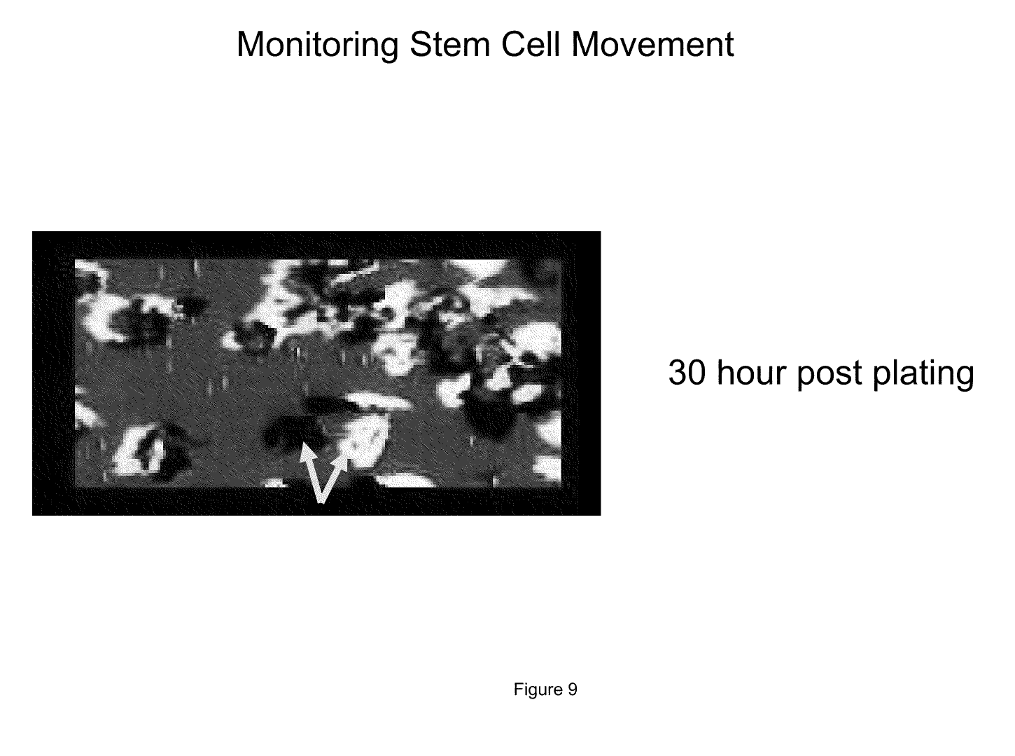

FIG. 8A shows rat MSC cells shortly after adding the cells to the colorimetric resonant reflectance biosensor and after 16 hours on the biosensor (FIG. 8B). The cells have spread out after 16 hours on the biosensor. FIG. 9 shows movement of rat MSC cells over 30 hours on the colorimetric resonant reflectance biosensor surface. The arrow on the left (pointing to a dark spot) demonstrates where the cell was shortly after it attached to the biosensor surface and the arrow on the right (pointing to a light spot) demonstrates where the cell was 30 hours after attachment to the biosensor surface.

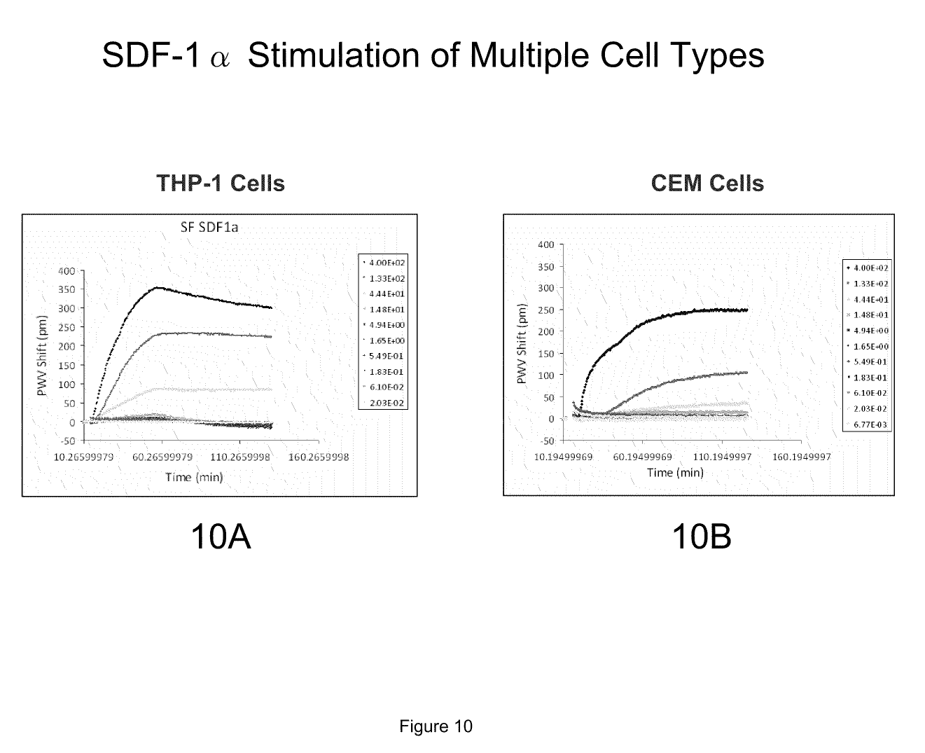

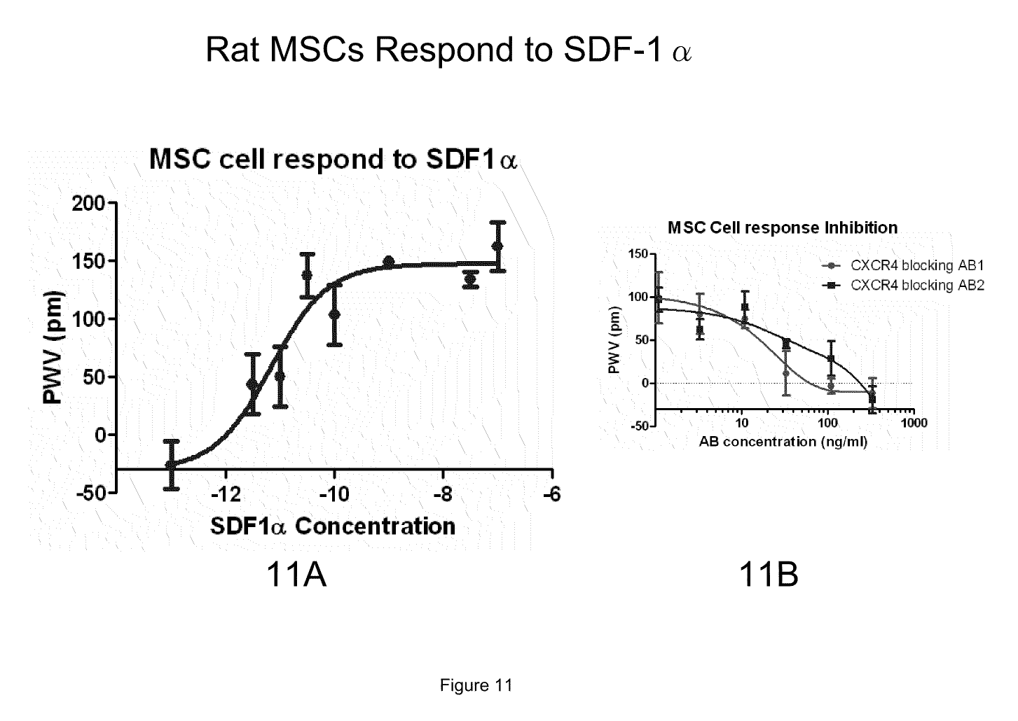

SDF-1α binds to and activates CXCR4, a GPCR. Stem cells will move to tissues releasing gradients of SDF-1α. Damaged tissue releases elevated levels of SDF-1α resulting in increased migration of mesenchymal stem cells to sites of injury. Chemotactic factors induce significant changes in the actin cytoskeleton of cells upon receptor activation. These changes are manifested as directional movement when the chemokine is presented as a gradient. SDF-1α induces the migration of mesenchymal stem cells and osteoblast progenitor cells. Overexpression of CXCR4 results in improved MSC migration and homing to sites of vascular injury. FIG. 10A shows the response of THP-1 cells and OEM cells (FIG. 10B) to different concentrations of SDF-1α using a colorimetric resonant reflectance biosensor microwell plates and a BIND® READER. SDF-1α induces a rapid and robust response in multiple cell types as measured with the BIND® READER. FIG. 11A shows the response of MSC cells to SDF-1α on colorimetric resonant reflectance biosensor microwell plate. FIG. 11B shows the response of MSC cells (7,000 cells in a 384 well microplate) to SDF-1α and inhibitors (CXCR4 blocking antibodies).

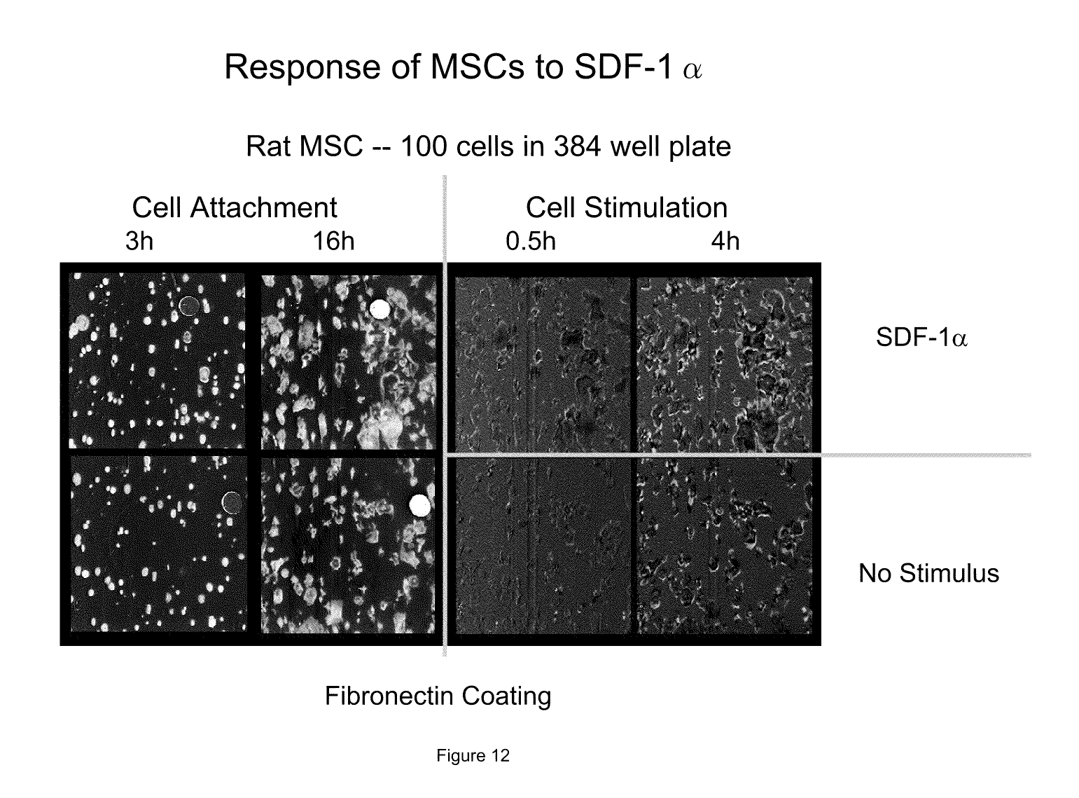

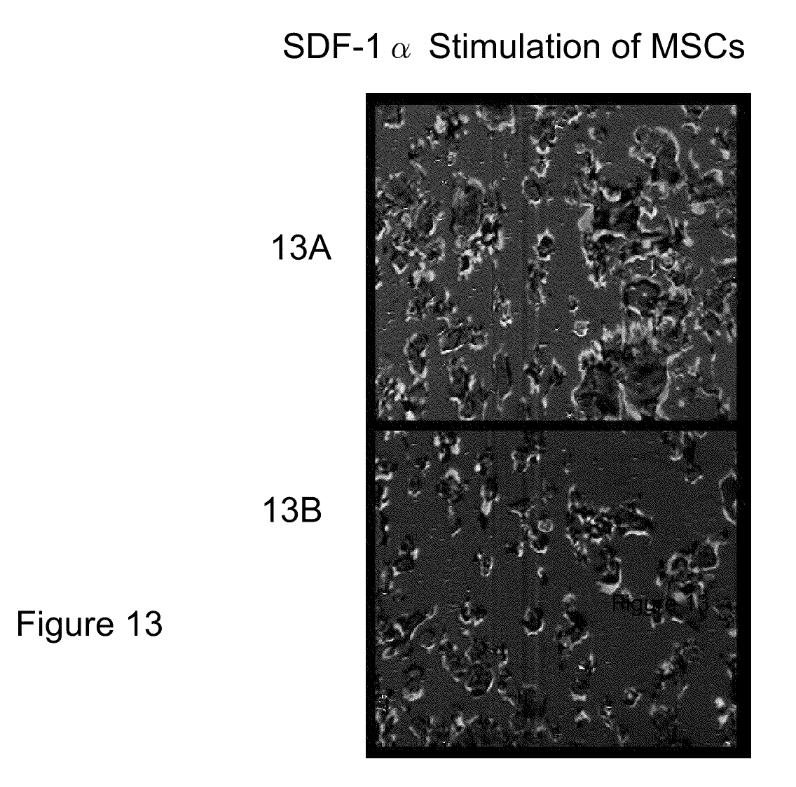

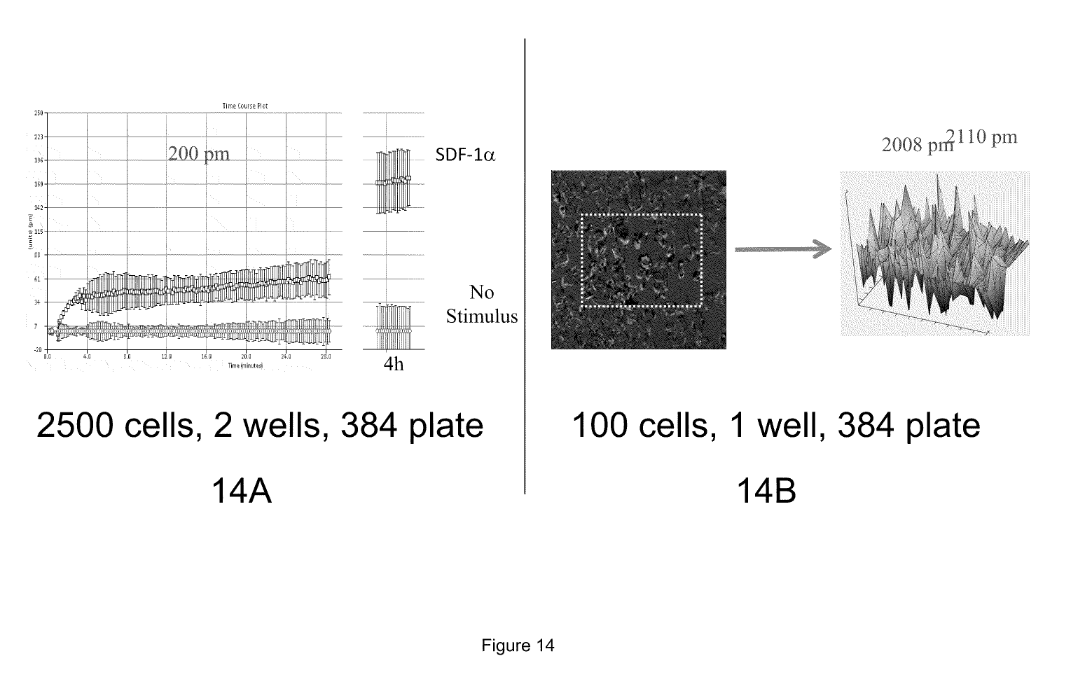

Rat MSC cells were added to a biosensor coated with fibronectin. Cell attachment was detected on a colorimetric resonant reflectance biosensor at 3 hours and 16 hours. See FIG. 12, left panels. The attachment signal was zeroed out and the cells were stimulated with SDF-1α or were not stimulated. See FIG. 12, right panels. Movement of the cells can be seen in the right panels of FIG. 12. The darker spots are where the cells were prior to detection and the lighter spots are where the cells are when the reaction was detected. Where no stimulus was added to the cells, some movement of the cells can be seen; however, where SDF-1α was added to the cells movement of the cells is seen along with a spreading out of the cells on the biosensor. FIG. 13A-B shows an enlargement of the right panels of FIG. 12. An enhanced signal can be seen on the cell edges where movement and/or cell adhesion is occurring. The enhanced signal correlates with the leading edge of the cells as they move across the biosensor as evidenced by time lapse imaging. This is consistent with extracellular matrix-integrin engagement of the cells. FIG. 14 demonstrates the reading from the BIND® READER (FIG. 14A) and the BIND®SCANNER (FIG. 14B). An approximately 7 to 10 fold improvement in signal to noise is observed.

Stem cell differentiation is dependant on cell adhesion (see Stem Cells 25:3005 (2007); Cardiovascular Res. 47:645 (2000)). By monitoring adhesion, differentiation can be detected. Label-free methods of imaging stem cells provide a unique opportunity to observe cell adhesion, movement, and differentiation. Different reactions of stem cells (e.g., different differentiation, chemotaxis, or adhesion) can be induced by different nanostructured regions occurring on one biosensor surface of the invention. U.S. Ser. No. 12/218,096 (PCT/US09/03541) describes biosensors with more than one type of grating sector on one biosensor surface. That is, two or more distinct spatial regions of gratings that exhibit different resonance values or periods (PWV1, PVW2, . . . ) occur on one biosensor surface. In one embodiment of the invention, the distinct spatial regions have sufficient spectral separation in response to illumination of the biosensor with light whereby the spectral separation can be resolved by a detection instrument reading the test device. Biosensors with two or more distinct spatial regions can be used to induce differentiation, movement or adhesion of stem cells or other cells. This differentiation, movement or adhesion can then be detected on the biosensor by detecting differing PWVs in each sector. For example, a cell population may differentiate on one sector with a unique resonance value as exhibited by an increase in PWV at that sector, but not differentiate on another sector as exhibited by no change in PWV at that sector.

Additionally, biosensors with two or more sectors, each comprising a different resonance values can be used to detect the response of two or more cell populations to one or more test reagents or stimuli in one vessel. For example, one cell type can be placed in one sector and a second cell type can be placed in a second sector with different a resonant value than the first sector. PWV's for each sector can be detected and the response of each cell type to the test reagent or stimuli can be determined in one vessel.

Also, the movement of cells from one sector to a second sector can be determined. For example, a chemoattractant can be placed on one sector and a cell population can be placed in a second sector. The movement of cells from the second sector to the sector with the chemoattractant can be detected by measuring PWVs for each sector. A decrease in PWV in the second sector and an increase in PWV in the sector with the chemoattractant demonstrates movement of the cells toward the chemoattractant sector.

### Methods of Screening Compounds for Effect on Differentiation of Cells

Methods of the invention can be used to screen compounds for their effect on differentiation of cells, including, for example, stem cells such as mesenchymal stem cells, hematopoietic stem cells, neuronal stem cells, and embryonic stem cells. Stem cells are cells that can renew themselves indefinitely while producing cell progeny that mature into more specialized, organ specific cells. Cell differentiation is the process by which a less specialized cell becomes a more specialized cell type. Differentiation can change the cell's size, shape, membrane potential, metabolic activity, and responsiveness to signals. For example, test compounds can maintain stem cell self-renewal, encourage or speed differentiation, slow or stop differentiation, or cause pluripotent cells to differentiate into different cells than normally observed. Test compounds can also encourage cells to de-differentiate. De-differentiation is where a partially or terminally differentiated cell reverts to an earlier developmental stage. Methods of the invention can detect the effects of test compounds on self-renewal, differentiation and de-differentiation of cells directly or indirectly by detecting changes (increase, decrease, or inhibition) of cell differentiation products or by detecting changes, for example, morphological changes, cell attachment to the biosensor changes, kinetic profile changes or other changes disclosed herein, in cells that have undergone self-renewal, differentiation or de-differentiation. That is, changes in cells (e.g., morphological changes, cell attachment to the biosensor changes, kinetic profile changes, or other changes disclosed herein) detected using methods of the invention can be used to determine the self-renewal, differentiation and de-differentiation of cells and to determine increases, decreases, or inhibition of cell differentiation in a cell population or mixed cell population.

Mesenchymal stem cells (MSCs) possess significant clinical potential as multipotent cells capable of self-renewal that can differentiate into several cell types, including, e.g., osteoblasts, chondrocytes and adipocytes. Methodologies of the invention provide label-free assays using optical resonance detection technology to enable high throughput screening of MSC (and other cells) migration and differentiation. MSCs can be readily propagated on, e.g., extracellular matrix-coated optical biosensors and respond to a bath application of chemokines with robust, dose-dependent, and highly sensitive label-free responses. MSC-osteoblast differentiation detection is characterized by unique label-free signals as collagen or mineral deposits are formed on the sensor surface. The real-time readout displays complete differentiation phenotypes in a single well, is more sensitive than traditional staining reagents, and can be applied in high-throughput for screening compound libraries, including small molecule libraries or siRNA libraries to monitor increases or decreases in the rate of differentiation or self-renewal.

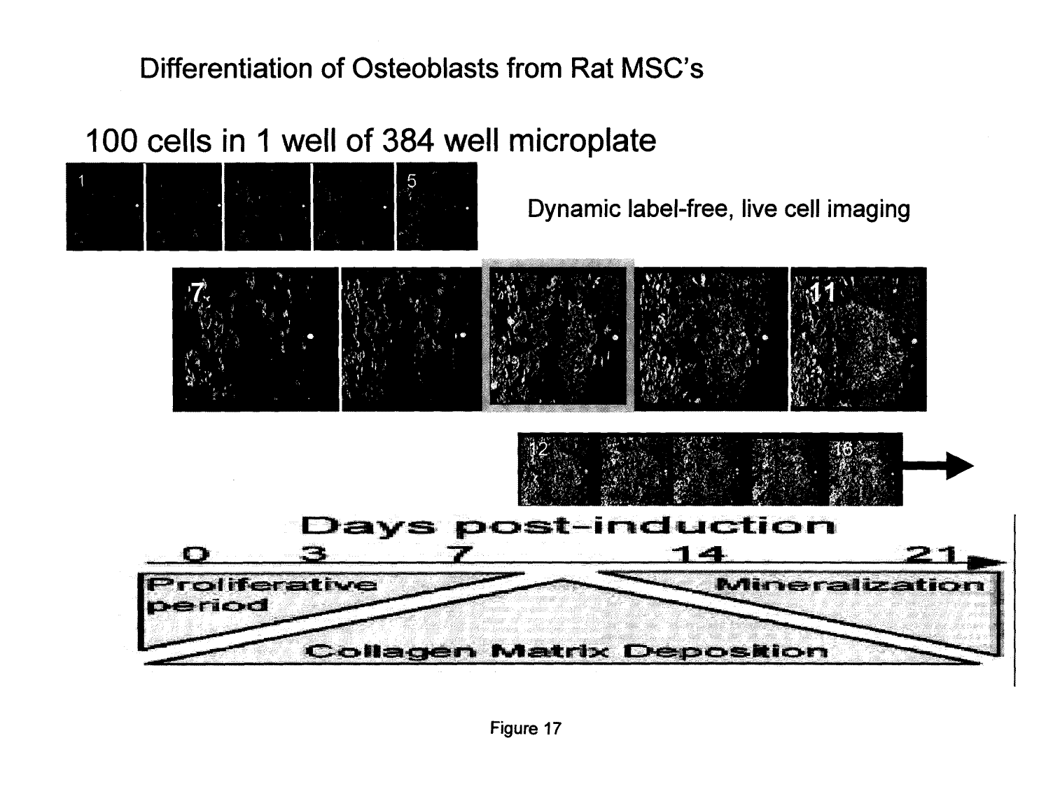

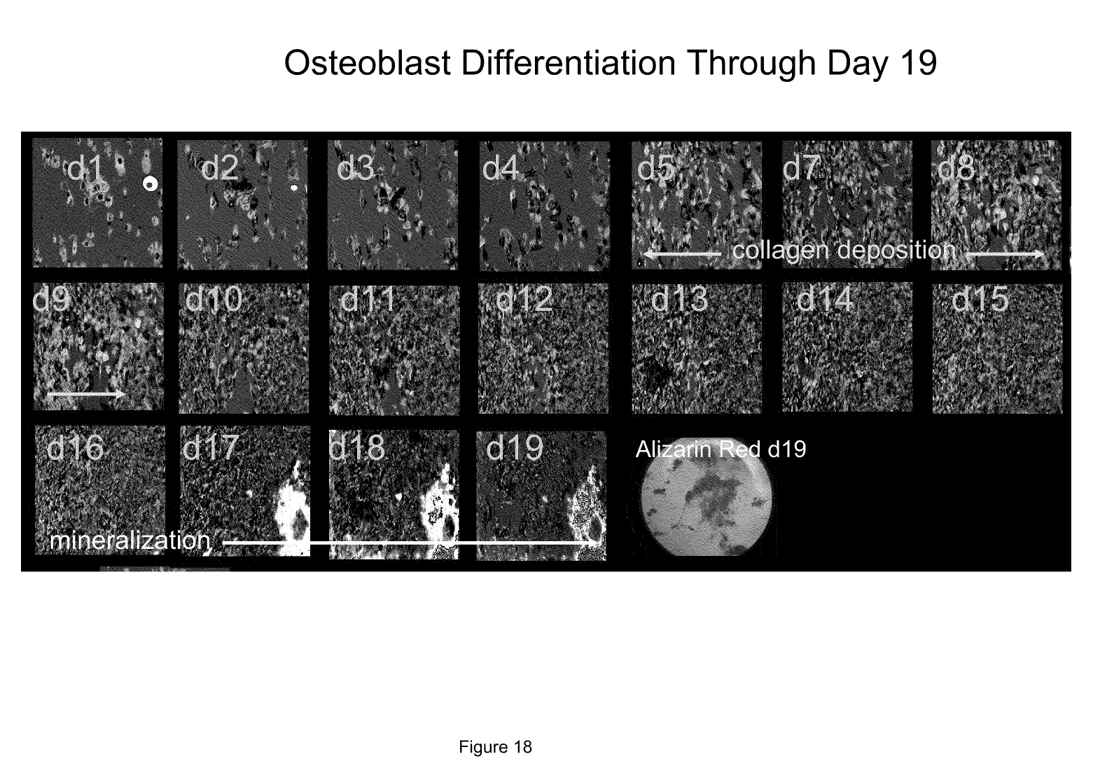

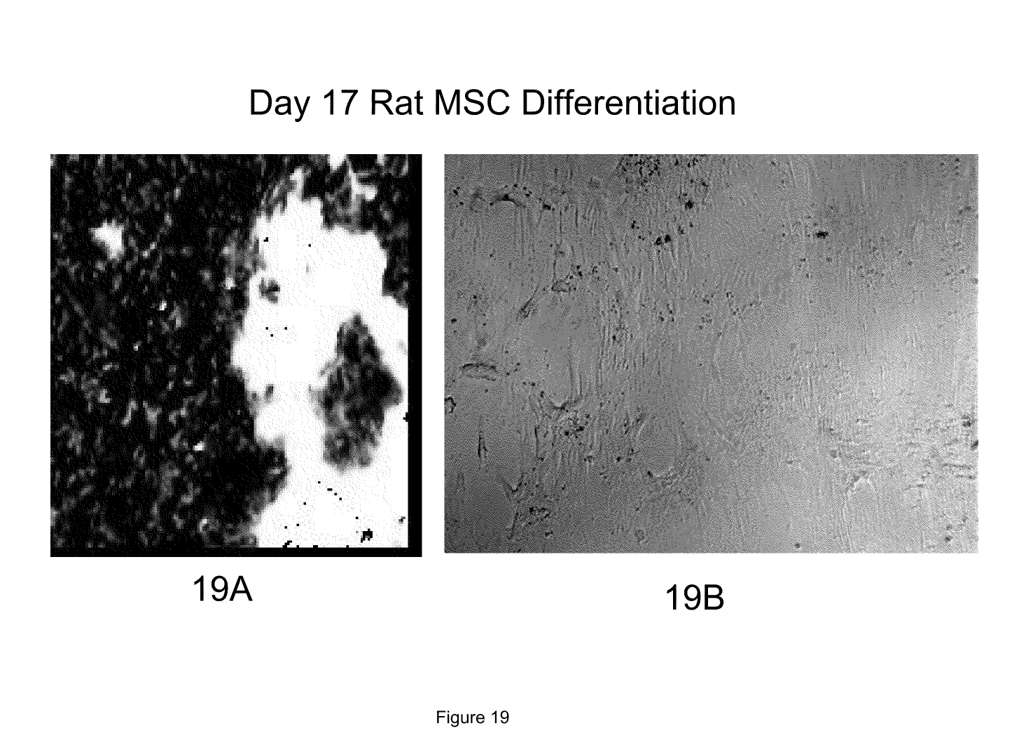

Rat MSCs can differentiate into, e.g., adipocytes, chondrocytes and osteoblasts. On a biosensor coated with collagen rat MSCs were induced to differentiate into osteoblasts. FIGS. 17 and 18 show that by day 14 the cells were mineralizing and producing bone. Alizarin red dye was used to confirm that the cells were indeed producing bone. See FIG. 18. The images in FIG. 18 were baselined from the previous day. The colorimetric resonant reflectance biosensor was put on the BIND® SCANNER for day to day readings. FIG. 19A shows a close up of the day 17 panel from FIG. 18. The white area is mineralization of the osteoblasts. FIG. 19B shows a phase contrast micrograph of the same portion of cells. The phase contrast micrograph does not show the differentiation of the cells. Therefore, the methods of the invention can detect the stage of differentiation of cells with no label or no stain.

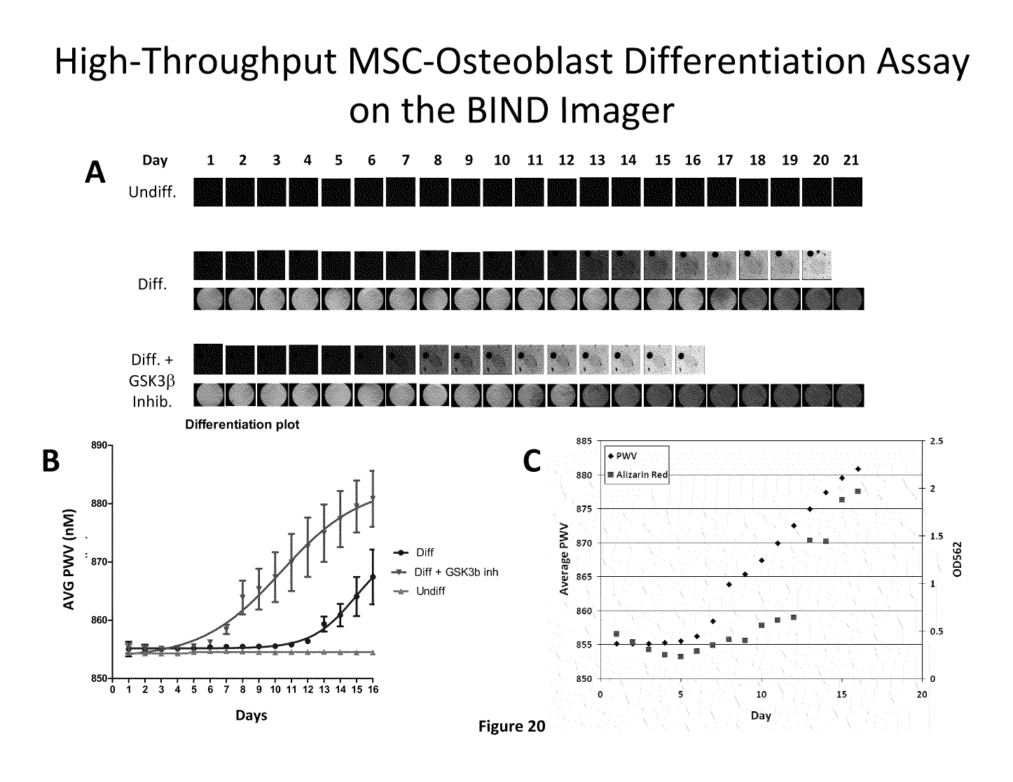

Rat MSCs (Invitrogen) were seeded in 384-well colorimetric resonant reflectance biosensors at 100 cells/well and treated with osteoblast differentiation media. Daily images were acquired on the BIND® SCANNER and baselined to the Day 0 cell attachment signal. A gradual and robust PWV shift (˜25 nM) was detected as bone-like minerals are deposited on the sensor surface, as indicated by alizarin red staining of parallel wells. See FIG. 20A. An inhibitor of glycogen synthase kinase 3 (GSK3β) expedites MSC-osteoblast differentiation. FIG. 20B demonstrates the detection of the expedited differentiation caused by GSK3β. FIG. 20C demonstrates that the BIND® SCANNER is more sensitive than alizarin red staining in detecting mineralization. Advantageously the BIND™ images shown in FIG. 20A are from single wells imaged on multiple days; alizarin red requires one well/day as an endpoint staining assay. Therefore, the methods of the invention allow the same well of cells to be assayed over several days, while the cell staining methods require the use of multiple wells over several days.

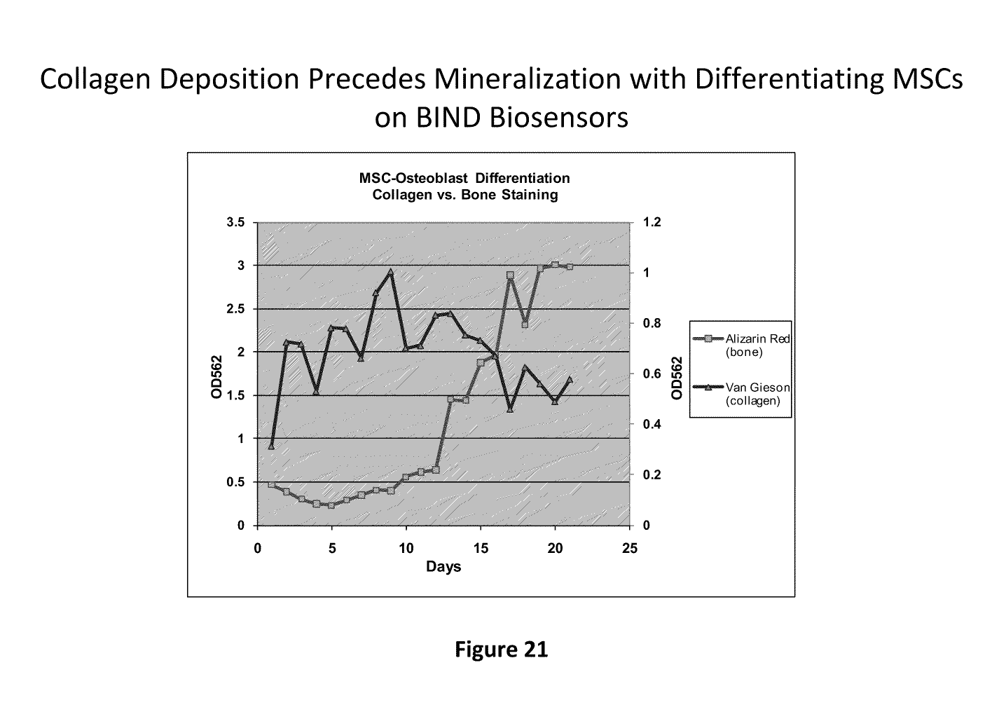

In another experiment rat MSCs were differentiated into osteoblasts on 384-well biosensors and stained daily for mineralization with alizarin red or collagen with Van Gieson's stain. Staining was quantitated with a plate reader at 562 nm. Collagen formation is shown to precede mineralization in differentiating MSCs on BIND™ biosensors consistent with normal bone formation. See FIG. 21.

Scanning electron microscopy (SEM) analysis of sensors with undifferentiated MSCs clearly reveal the underlying grating structure, whereas sensors with differentiated MSCs are coated with a layer of mineralization nodule deposits that obscures the grating—consistent with the diffuse but strong PWV shifts measured across the well. Energy dispersive X-ray (EDS) analysis of larger deposit clusters indicates the presence of calcium (Ca) and phosphorous (P), consistent with bone deposition. The titanium (Ti), oxygen (O), and silicone (Si) peaks derive from biosensor components.

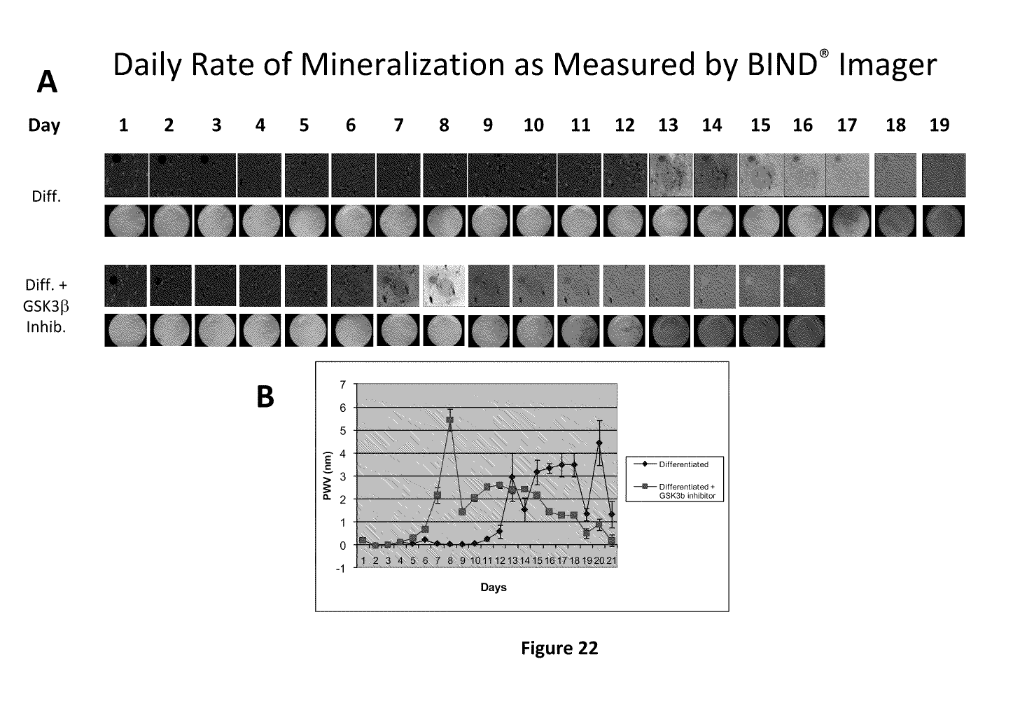

In another experiment rat MSCs (Invitrogen) were cultured in osteoblast differentiation media with or without GSK3β inhibitor for 1 to 19 days. BIND™ images were collected daily and baselined to previous day measurements, thus providing information on the rate of mineralization. It is not possible to collect rate of mineralization data with standard staining methodologies such as alizarin red. See FIG. 22A. FIG. 22B shows the quantitation of PWV shifts as measured on BIND® SCANNER (+/− standard deviation, n=12 wells). The distinct rate of differentiation with the GSK3β inhibitor suggests that GSK3β regulates the timing and rate of MSC differentiation into osteoblasts.

Stem cells possess significant clinical potential and the methodologies of the invention provide label-free assays using optical resonance detection technology to enable high throughput screening of stem cell migration and differentiation. Full differentiation profiles are available from single cell culture well and rates of differentiation can be determined.

One embodiment of the invention provides a method for screening a candidate compound for its ability to modulate cell differentiation. One or more types of cells (homogenous or heterogeneous cell populations) are added (with or without ECM) to a surface of a colorimetric resonant reflectance biosensor (or a grating-based waveguide biosensor), which can be optionally coated with ECM. In one embodiment, different ECM's or materials that putatively support cell attachment and differentiation can be applied onto a sensor as a screen for those materials that accentuate differentiation or other cell processes (adhesion, movement, etc). The cells can be induced to differentiate. A change in cell differentiation in the presence or absence of the candidate compound is detected by comparing the peak wavelength values (or refractive indices) of each cell population in the presence or absence of the candidate compound. A change in cell differentiation activity in the presence of the candidate compound relative to cell differentiation activity in the absence of the candidate compound indicates an ability of the candidate compound to modulate cell differentiation. The change in cell differentiation activity can be an increase in cell differentiation activity, decrease in cell differentiation activity, inhibition of cell differentiation activity, a change in the type of differentiated cell (that is, the test compound causes the cell to differentiate into a cell type not normally observed). The change in cell differentiation activity can be an increase or decrease in collagen production, an increase or decrease in mineralized nodule formation, or an increase or decrease in other cell product of differentiation. The one or more types of cells can be stem cells, such as mesenchymal stem cells. The change in cell differention activity can be detected by detecting a change in cell size, cell shape, cell adhesion, cell membrane potential, cell metabolic activity, or cell responsiveness to signals.

Another embodiment of the invention provides a method for screening a candidate compound for its ability to modulate cell differentiation. One or more types of cells can be added (with or without ECM) to a surface of a colorimetric resonant reflectance biosensor (or a grating-based waveguide biosensor), which can optionally be coated with ECM. The one or more types of cells can be induced to differentiate. The production of one or more cell products of differentiation are detected in the presence or absence of the candidate compound by comparing the peak wavelength values (or refractive indices) in the presence or absence of the candidate compound. A change in one or more cell products of differentiation in the presence of the candidate compound relative to one or more cell products of differentiation in the absence of the candidate compound indicates an ability of the candidate to modulate cell differentiation.

### Methods of Detecting Gene Modulation of Cell Differentiation

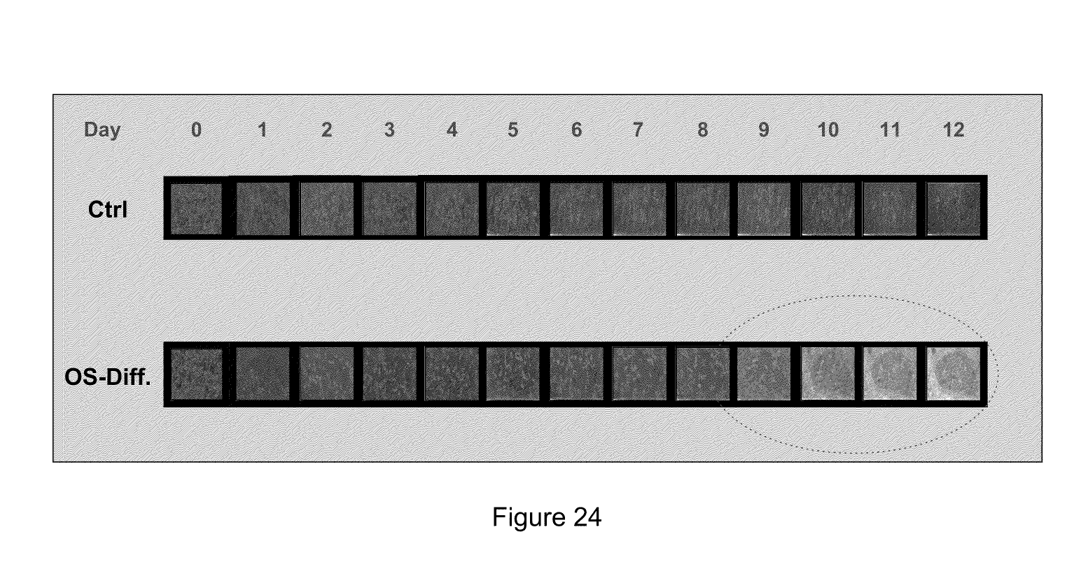

Inhibition of GSK3β or adenosine kinase (ADK) accelerates MCS-osteoblast differentiation. Activation of cAMP by forskolin treatment slows down osteoblast differentiation. Human MSCs were seeded on a 384-well colorimetric resonant reflectance biosensor plate. The cells were treated with an osteoblast differentiation cocktail. PWVs were measured daily. Representative wells from untreated cells (Ctrl) and osteoblast-differentiated (OS-Diff) cells are shown in FIG. 24. Mineralization deposits on the sensor surface begin to appear on day 9 for the osteoblast differentiated cells and continue to accumulate thereafter. The accumulation of mass on the surface of the biosensor results in a very large and robust positive PWV signal shift.

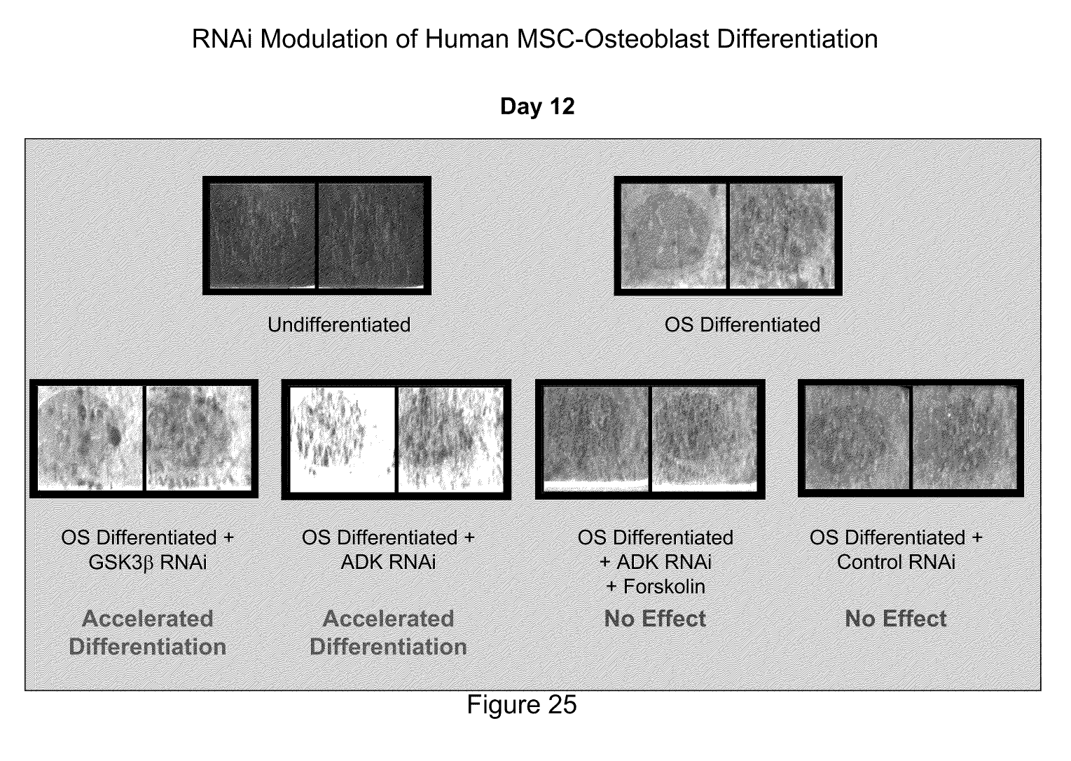

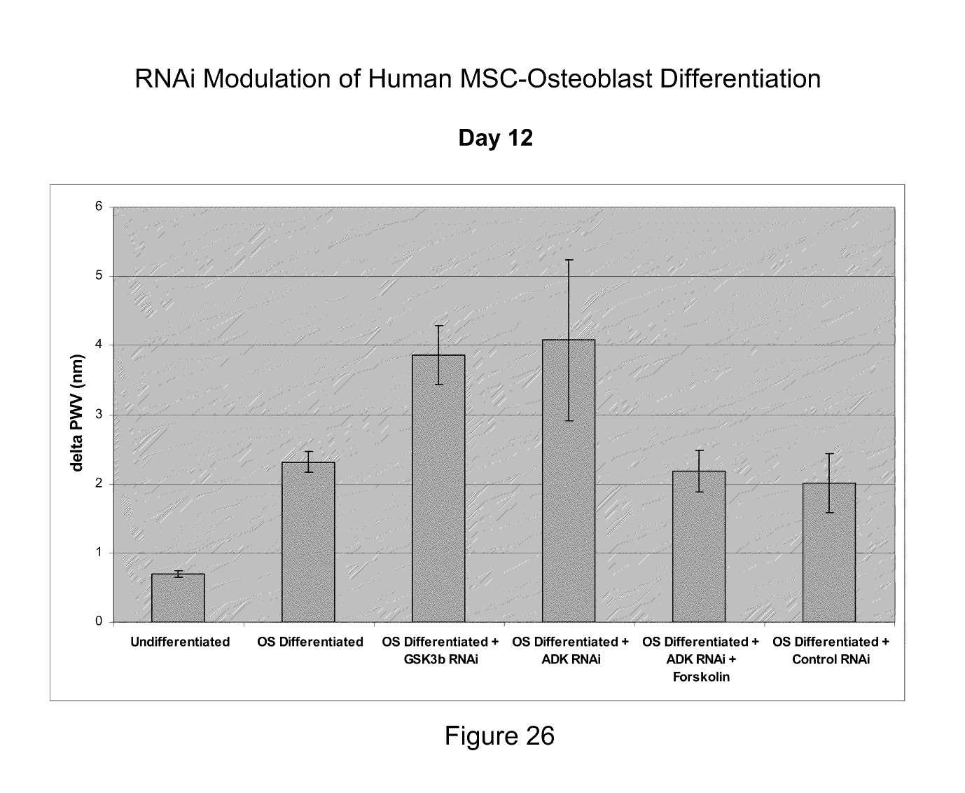

siRNA molecules that are specific for GSK3β or ADK were purchased from ThermoFisher and transfected into the osteoblast differentiated cells. Accelerated osteoblast differentiation was detected in label-free assays on the BIND® SCANNER when siRNA molecules specific for GSK3β and ADK were transfected into human MSCs just prior to differentiation. See FIGS. 25 and 26. FIG. 25 shows sample wells at day 12 for several treatment conditions. FIG. 26 quantifies the results shown in FIG. 25. 6 wells per treatment condition were averaged. In the case of the ADK siRNA treatment, the accelerated differentiation phenotype could be blocked by incubating the cells in forskolin following ADK siRNA transfection. ADK is a critical upstream enzyme in the adenylate cyclase-cAMP signal transduction pathway. When ADK is down-regulated by siRNA transfection the signal transduction pathway gets inhibited leading to the acceleration phenotype. Forskolin, however, activates the same signal transduction pathway downstream of ADK, therefore forskolin treatment restores proper signal transduction and blocks the effects of ADK down-regulation.

These experiments demonstrate that the methods of the invention can be used for detecting and assessing specific gene expression modulation by, for example, inhibitory nucleic acids or other gene modulation methods. Inhibitory nucleic acids include, for example, triplex forming nucleic acids, piRNA, dsRNA, siRNA, hairpin dsRNA, shRNA, miRNA, ribozymes, aptazymes, and antisense nucleic acids.

### Cell Migration Assays

Cell migration in response to environmental stimuli is central to a broad range of physiological processes, including immune responses, wound healing, and stem cell homing. In some cases, excessive cell migration can contribute to disease pathologies, including inflammatory diseases and tumor metastasis. Drug discovery efforts for inhibitors of cell migration are hampered by the lack of high throughput assays to enable primary screening campaigns in functionally relevant cell types. The invention provides different high throughput screening assays for chemotaxis using label-free optical biosensor technology. The BIND™ “touchdown” assay measures the invasion of cells through a collagen layer and onto the biosensor surface coated with chemokine. The BIND™ “lift-off” assay measures the detachment of cells away from the collagen-coated sensor surface toward chemokine presented in the bath media. Both assays are independent of transwells, require low cell numbers per well, and are 1536-well compatible.

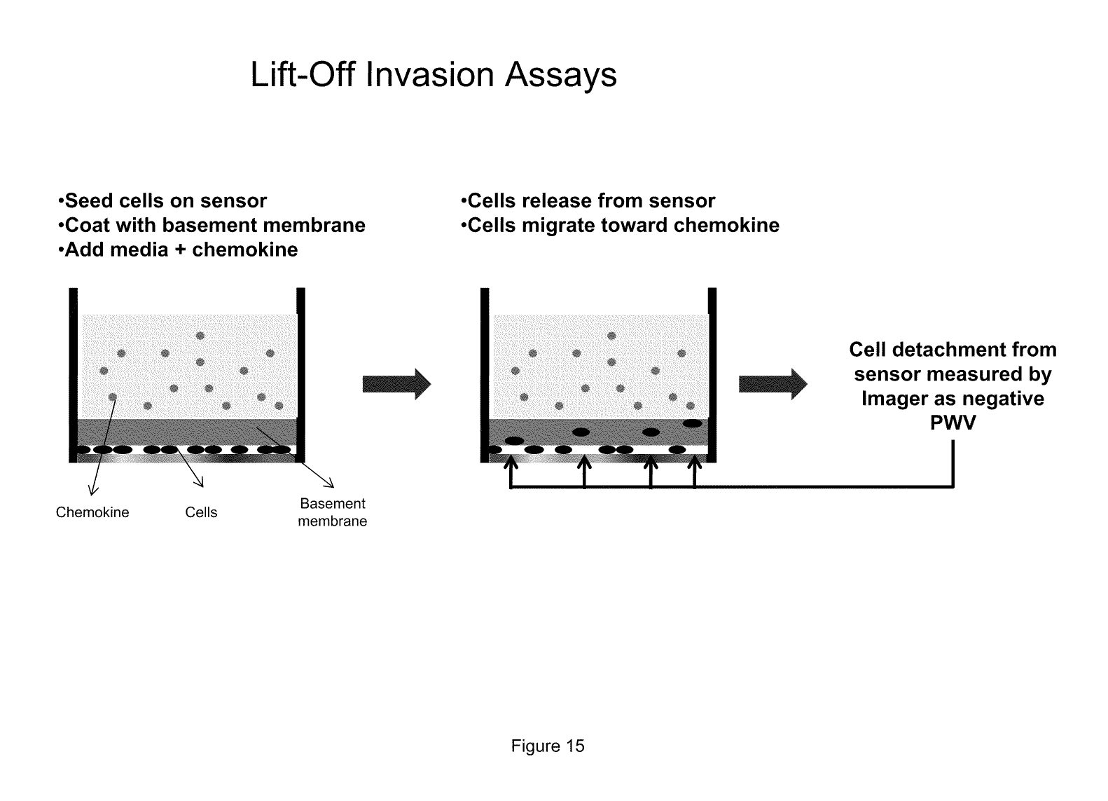

The “lift-off” assay provides a method of detection of responses of a first population of cells to one or more stimuli. See FIG. 15. The cells can be any type of cell, including, e.g., stem cells. One or more extracellular matrix ligands can be immobilized to a surface of a colorimetric resonant reflectance biosensor or a grating-based waveguide biosensor. The first population of cells have cell surface receptors specific for the one or more extracellular matrix ligands. The first population of cells can then be added to the biosensor. Alternatively, the first population of cells is mixed with one or more extracellular matrix ligands, wherein the first population of cells have cell surface receptors specific for the one or more extracellular matrix ligands. The first population of cells and the one or more extracellular matrix ligands are added to a surface of a colorimetric resonant reflectance biosensor or a grating-based waveguide biosensor.

A gel or gel-like substance can be added to the biosensor so that the first population of cells and extracellular matrix ligands are partially or totally covered by the gel or gel-like substance.

The gel or gel-like substance can be, e.g., MATRIGEL™ basement membrane matrix, alginate gel, collagen gel, agar, agarose gel, synthetic polymer hydrogel, synthetic hydrogel matrix, laminin gel, vitrogen, chitosan gel, fibrinogen gel, PuraMatrix™ peptide hydrogel is a (synthetic matrix that is used to create defined three-dimensional (3D) micro-environments for a variety of cell culture experiments), or gelatin. The gel or gel-like substance can optionally comprise one or more ECM ligands, chemotactic agents, growth factors, specific binding partners, ligands, or combinations thereof.

Alternatively, instead of a gel or a gel-like substance, a second population of cells or artificial basement membrane can be added to the biosensor surface. The second population of cells, artificial basement membrane, or gel or gel-like substance, can form a barrier through which the first population of cells migrate. Artificial basement membranes are well known in the art. See, e.g., Inoue et al., J. Biomed. Mater. Res. A. 73:158 (2005); Guo et al., Int. J. Mol. Med. 16:395 (2005); Saha et al., Ind. J. Exp. Biol. 43:1130 (2006); Barroso et al., J. Biol. Chem., 283:11714 (2008); Okumoto et al., J. Hepatol., 43:110 (2005). A second population of cells can be, e.g., epithelial cells or a population of endothelial cells.

One or more stimuli can be added to the gel, gel-like substance, second population of cells or artificial basement membrane. The one or more stimuli can be, for example, a chemotactic agent, a ligand, or a third population of cells that produce stimuli.

The responses of the first population of cells to the test reagents or stimuli are detected. If the first population of cells move away from the surface of the biosensor PWVs or effective refractive index will demonstrate a reduction. The responses of the first population of cells to the one or more stimuli or test reagents can be detected by monitoring the peak wavelength value over one or more time periods or by monitoring the change in effective refractive index over one or more time periods. The responses of the first population of cells can be detected in real time. Additionally, the responses of the second population of cells to the stimuli or test reagents can be detected relative to the first population of cells. The responses of the second population of cells to the one or more stimuli or test reagents can be detected by monitoring the peak wavelength value over one or more time periods or by monitoring the change in effective refractive index over one or more time periods. The responses of the second population of cells can be detected in real time.

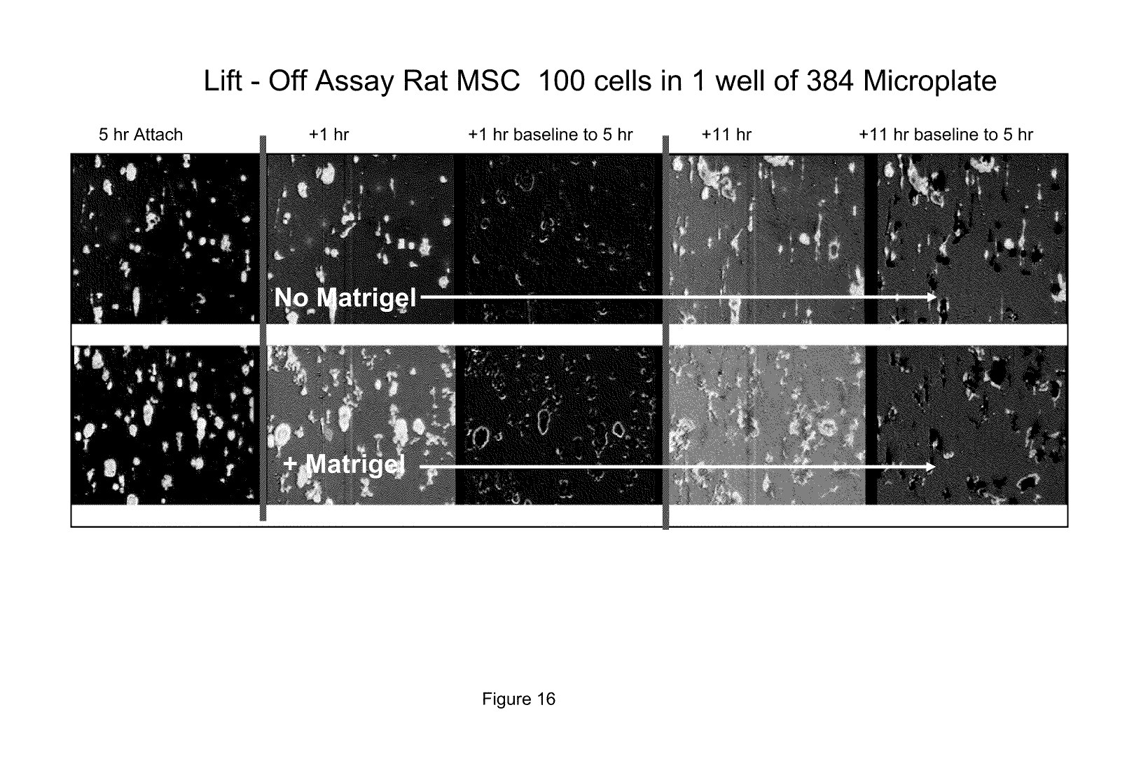

For example, HT1080 cells respond to fetal bovine serum (FBS) while NIH3T3 cells do not. HT1080 cells and NIH3T3 cells were exposed to FBS in a chemokine in a lift off assay using a colorimetric resonant reflectance biosensor. HT1080 cells lifted off of the biosensor and moved towards the FBS as demonstrated by less signal from the biosensor. NIH3T3 cells remained on the biosensor surface and proliferated because they do not react to FBS as demonstrated by more signal from the biosensor. FIG. 16 shows MSC cells lifting up off the biosensor in the presence of MATRIGEL™ basement membrane matrix as compared to control wells. The MSC attachment signal can be readily identified with the MATRIGEL™ coating. The MSCs display a tendency to lift up off the sensor as compared to control wells. This is evidenced by a negative PWV shift displayed as black in FIG. 16.

The “touchdown” assay provides methods of detection of responses of a first population cells, such as stem cells or primary cells, to one or more stimuli. One or more stimuli are added to a surface of a colorimetric resonant reflectance biosensor or a grating-based waveguide biosensor. A gel, gel-like substance, second population of cells or artificial basement membrane is added to the biosensor surface. The first population of cells is mixed with one or more extracellular matrix ligands, wherein the first population of cells have cell surface receptors specific for the one or more extracellular matrix ligands. The first population of cells is added to the biosensor. The responses of the first population cells to the one or more stimuli are detected. If the first population of cells moves toward the surface of the biosensor the PWVs or effective refractive index will increase. The responses of the first population of cells to the one or more stimuli can be detected by monitoring the peak wavelength value over one or more time periods or by monitoring the change in effective refractive index over one or more time periods. The responses of the first population of cells can be detected in real time. The one or more stimuli can be a chemotactic agent or a third population of cells that produce stimuli. The second population of cells can be a population of epithelial cells or a population of endothelial cells. The first population of cells can be a population of stem cells.

### Other Assays

Chemotactic agents can be applied to cells in a “bath application.” In this method cells, such as stem cells or primary cells, are adhered to the biosensor and subsequently treated with a chemotactic agent. Cell response (random movement and adhesion) is detected using the BIND® READER or BIND® SCANNER in real time following addition of the test agent.

Directional migration of stem cells to a chemotactic gradient in two dimensions can be detected using methods of the invention. In these methods, cells, such as stem cells are adhered to the biosensor, preferably in a corner or side of a microtiter well. Chemotactic agents are added from a defined area in the well in such a manner as to create a gradient of concentration of the chemotactic agent across the well. Cell response and migration are detected on the BIND® READER or BIND® SCANNER. Directionality can be determined in a number of ways; in one embodiment, the BIND® READER or BIND® SCANNER is capable of measuring individual sectors within the microtiter well, such that changes can be monitored as cells migrate from one sector to another. In another method, the grating of the biosensor is patterned with different grating structures that resonate to different light frequencies on the biosensor. By monitoring the different sectors utilizing different light frequencies, one can monitor movement of cells from one sector to another. In a third method, the BIND® SCANNER, through single cell analysis, can directly measure and track movement of individual cells through their adhesion changes on the biosensor.

Another mode of stem cell analysis relates to use of the label-free detection platform to detect stem cell differentiation. In one embodiment, the microplate biosensors are coated with extracellular matrix material and subsequently incubated with stem cells. The stem cells adhere to the extracellular matrix and subjected to culture conditions that promote stem cell differentiation. In some cases, test agents may be added to detect their influence on stem cell differentiation. Differentiation can be followed using the BIND® READER or BIND® SCANNER by detecting the PWV signal at different time intervals. Conversely, one may use the PWV signal to monitor stem cell division under conditions meant to prevent differentiation. In another mode, the attachment signal of differentiated cells may be qualitatively/quantitatively different than the undifferentiated cells based on differential interaction with ECM. This difference can be utilized as a signature of the different cell types on the BIND® SCANNER.

In another embodiment of the invention it can be desirable to monitor the stage of differentiation through a process described as “biological profiling.” Biological profiling is conceptually related to genetic profiling using gene chips, in that patterned responses can be monitored based on biological responses within cells. Biological profiling differs, however, in that it uses live cells and can be monitored in real time. In this method, stem cells are adhered to extracellular matrix, and stem cells are attached. Subsequently, stem cells are subjected to differentiation conditions, ligands, or test compounds or environmental conditions. The biological profile is a collection of about 2, 5, 10, 20, 50 or more PWVs of a cell population taken over time (about 1, 5, 10, 30, 60 seconds, about 1, 2, 3, 4, 5, 10, 20, 40, 60 or more minutes). The biological profile reveals changes in PWV over time and represents a unique signature of cells' reaction to differentiation conditions, test compounds or environmental conditions. For example, where the test compound induces differentiation, the PWVs may rise over time as the cells differentiate and grow. Where the test compound is a toxin, the PWVs may decline over time as the cells become weaker and die. A biological profile can also be PWVs of a cell population taken for two or more differing concentrations of a test compound or ligand. The biological profile reveals changes in PWV over differing concentrations and represents a unique signature of the test compound or ligand.

Periodically, ligands are added to the cells to probe for label-free responses; for example, a panel of GPCR ligands is added to probe for a patterned response of the cells to the ligands. Different responses on the biosensors will emerge from the cells as they differentiate and new receptors are upregulated or downregulated. Further, proteins involved in signal transduction pathways or cell adhesion pathways will change in response to differentiation and will also cause changes in response to the panel of ligands. The panel of ligands, therefore, can be to specific receptors that are known to change in response to differentiation, or preferably, are more random modulators of cells. Each differentiated cell type, therefore, will give its own patterned response to the ligands, hence, a “biological profile”. Further, the optical biosensor can incorporate an array of electrical probes to provide electrical stimulation for differentiated cells that may respond to electrical potential e.g. muscle or nerve cells. The optical biosensor will record the response of such a cell to electrical stimulation. The BIND® SCANNER can monitor the geometric relationship between the responding cell and electrical probe. Similarly, the biosensor may comprise part of a flow device enabling stem cell assays involving flow or pressure.

### Methods of Increasing Sensitivity and Reducing Background Signal

Methods of the invention can be used to increase sensitivity of binding assays and decrease the background signal of binding assays. Binding assays can comprise immobilizing or otherwise associating a ligand or specific binding substance with a biosensor surface and then adding binding partners to the surface. Binding of the binding partner to the ligand or specific binding substance can be detected. However, in certain cases, the binding partners can non-specifically bind to the biosensor. That is, the binding partners do not specifically bind to the ligands or specific binding substances, but to the biosensor surface itself. Non-specific binding can be reduced by using blocking agents. Blocking agents, however, can reduce the specific binding signal.

“Specifically binds,” “specifically bind” or “specific for” means that a binding partner recognizes and binds a specified ligand or specific binding substance, but does not substantially recognize or bind other non-specific molecules in the sample.

One embodiment of the invention provides a method for increasing the sensitivity of binding assays and decreasing the background of binding assays by adding a layer of a gel or gel-like substance over the specific binding substances or ligands that are immobilized or otherwise associated with the biosensor surface. The gel or gel-like substance can be, e.g., MATRIGEL™ basement membrane matrix, alginate gel, collagen gel, agar, agarose gel, synthetic polymer hydrogel, synthetic hydrogel matrix, laminin gel, vitrogen, chitosan gel, fibrinogen gel, gelatin or PuraMatrix™ peptide hydrogel (a synthetic matrix that is used to create defined three-dimensional (3D) micro-environments for a variety of cell culture experiments). The gel or gel-like substance can optionally comprise one or more ECM ligands, chemotactic agents, growth factors, specific binding partners, ligands, or combinations thereof.

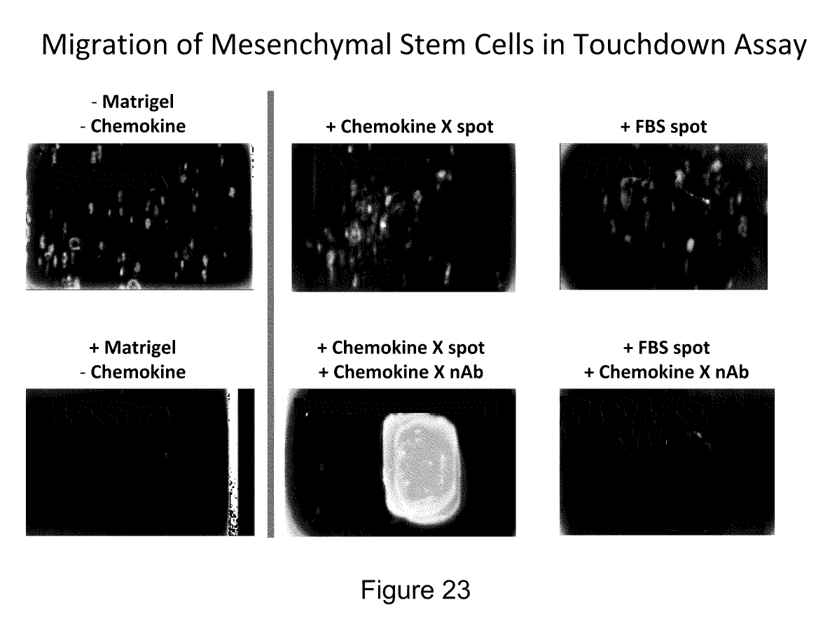

In a the “touchdown” chemotaxis assay a chemokine (PDGF-BB) was spotted in the center only of wells of a 384 well biosensor plate (instead of over the whole bottom surface of the well). The well surface was then coated with MATRIGEL™ basement membrane matrix and mesenchymal stem cells (MSCs) were added to the surface of the MATRIGEL™ basement membrane matrix. A BIND® SCANNER was used to detect peak wavelength values from the wells to determine if the MSCs would migrate preferentially toward the spot of PDGF-BB as opposed to randomly across the well. The cells were scored for migration as cell attachment (positive PWV shift) signal. The data indicate that there is, in fact, a bias of MSCs migrating toward the PDGF-BB spot. Parallel wells were also prepared and a neutralizing antibody specific for PDGF-BB was added to the well to determine if chemokine-induced migration could be blocked. See FIG. 23. The panel in the middle of the bottom row of FIG. 23 shows that the antibody does block MSC migration, but also shows a very bright oblong of positive PWV shift in the center of the well representing the interaction of PDGF-BB antibody with the PDGF-BB spotted on the biosensor. In FIG. 23, “chemokine X” is PDGF-BB; “chemokine X nAb” is neutralizing antibody specific for PDGF-BB.

It is surprising that the MATRIGEL™ basement membrane matrix has increased the sensitivity of the system by reducing background signal on the biosensor and generated an antibody-antigen signal:background response that is greater than predicted. Therefore, gels and gel-like substances provide a new type of biosensor surface chemistry, distinct from other biosensor surface chemistries or dextran-like biosensor surfaces, that provides an improvement for biochemical applications where signal:background requires optimization.

Therefore, the invention provides a colorimetric resonant reflectance biosensor grating surface or a grating-based waveguide biosensor grating surface comprising: one or more specific binding substances immobilized to or associated with the biosensor surface and a layer of a gel or gel-like substance over the one or more specific binding substances. The biosensor grating surface can form an internal surface of a liquid containing vessel. The liquid containing vessel can be a microtiter plate, or a microfluidic channel.

The invention also provides an improved method for detecting reactions between a specific binding substance and a binding partner on a colorimetric resonant reflectance biosensor grating surface or a grating-based waveguide biosensor grating surface comprising. One or more specific binding substances can be applied to the biosensor grating surface such that the one or more specific binding substances become immobilized to or associated with the biosensor grating surface. The one or more specific binding substances can be deposited at one or more distinct locations on biosensor surface. A gel or gel-like substance is added to the biosensor surface. Optionally, one or more ECM ligands, chemotactic agents or ligands can be added to the biosensor surface. One or more binding partners that potentially bind the one or more specific binding substances can be added to the gel or gel-like surface. The interaction of the one or more specific binding substances and the one or more binding partners is detected by determining one or more peak wavelength values or effective indices of refraction. The results are more specific than results obtained without the use of the gel or gel-like substance and the non-specific background is reduced as compared to results obtained without the use of the gel or gel-like substance. Without wishing to be bound to a particular theory, it is believed that the gel or gel-like substance functions to block non-specific binding resulting in more specific results.

One embodiment of the invention provides a kit comprising one or more colorimetric resonant reflectance biosensor grating surfaces or one or more grating-based waveguide biosensor grating surfaces and one or more containers of gel or gel-like substances. The kit can optionally contain one or more specific binding substances. The one or more colorimetric resonant reflectance biosensor grating surfaces or a grating-based waveguide biosensor grating surfaces can comprise one or more specific binding substances immobilized to or associated with the biosensor grating surface.

All patents, patent applications, and other scientific or technical writings referred to anywhere herein are incorporated by reference in their entirety. The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

We claim:

1. A method for detecting differential responses of two or more types of cells in one vessel to stimuli or a test reagent, wherein the two or more types of cells do not comprise detectable labels, comprising:
(a) applying the two or more types of cells to the one vessel, wherein the vessel comprises a colorimetric resonant reflectance biosensor surface, a grating-based waveguide biosensor surface, or a dielectric film stack biosensor surface, wherein the biosensor surface has one or more specific binding substances immobilized to its surface and wherein the one or more specific binding substances can bind one or more of the two or more types of cells;
(b) allowing the two or more types of cells to bind to the one or more specific binding substances; and
(c) detecting the differential responses of the two or more cell types.

2. The method of claim 1, wherein the differential responses are different times of the two or more types of cells to attach to the one or more specific binding substances.

3. The method of claim 1, wherein the differential responses are different cell attachment morphologies displayed by the two or more types of cells to the one or more specific binding substances.

4. The method of claim 1, wherein the differential responses are different strengths of attachment of the two or more cell types to the one or more specific binding substances.

5. The method of claim 1, further comprising:
(d) exposing the two or more cell types to one or more test reagents or stimuli; and
(e) detecting the differential responses of the two or more cell types.

6. The method of claim 5, wherein the differential responses are different strengths of response of the two or more cell types to the one or more test reagents or stimuli.

7. The method of claim 5, wherein the differential responses are different cell morphologies displayed by the two or more types of cells in response to one or more test reagents or stimuli.

8. The method of claim 5, wherein the differential responses are different cell responses of the two or more cell types to the one or more test reagents or stimuli over time.

9. The method of claim 5, wherein the differential responses are different response kinetics of the two or more cell types over time.

10. The method of claim 1, further comprising:
(a) exposing the two or more cell types to a first test reagent or first stimuli;
(b) detecting the responses of the two or more cell types to the first test reagent or first stimuli;
(c) exposing the two or more cell types to a second test reagent or second stimuli, wherein the response of one of the cell types in the two or more cell types to the second test reagent or second stimuli is known;
(d) detecting the responses of the two or more cell types to the second test reagent or second stimuli;
(e) identifying on the biosensor the one of the cell types in the two or more cell types that have a known response to the second test reagent or second stimuli;
(f) detecting the differential response of the two or more types of cells.

11. The method of claim 1, wherein the one or more test reagents or stimuli are expressed by one or more cells of the two or more types of cells present on the biosensor surface.

12. A method of detecting the presence of a first cell type in a mixed population of cells, wherein the cells in the mixed population of cells do not comprise detectable labels comprising:
(a) applying the mixed population of cells to one vessel, wherein the vessel comprises a colorimetric resonant reflectance biosensor surface, a grating-based waveguide biosensor surface, or a dielectric film stack biosensor surface, wherein the biosensor surface has one or more specific binding substances immobilized to its surface;
(b) allowing the mixed population of cells to bind to the one or more specific binding substances, wherein the first cell type has a differential response from the other cells of the mixed population of cells to binding to the one or more specific binding substances; and
(c) detecting differential responses of the mixed population of cells, wherein the presence of the first type of cells is detected by their differential response.

13. The method of claim 12, wherein the differential response is a different time of the first cell type to attach to the one or more specific binding substances.

14. The method of claim 12, wherein the differential response is a different cell attachment morphology displayed by the first type of cells to the one or more specific binding substances.

15. The method of claim 12, wherein the differential response is a different strength of attachment of the first type of cells to the one or more specific binding substances.

16. The method of claim 12, wherein the percentage of the first type of cells in the mixed population of cells is determined.

17. The method of claim 12, wherein the differential response is a different response of the first type of cells over time.

18. A method of detecting the presence of a first cell type in a mixed population of cells, wherein none of the cells in the mixed population of cells comprise detectable labels comprising:
(a) applying the mixed population of cells to one vessel, wherein the vessel comprises a colorimetric resonant reflectance biosensor surface, a grating-based waveguide biosensor surface, or a dielectric film stack biosensor surface, wherein the biosensor surface has one or more specific binding substances immobilized to its surface;
(b) allowing the mixed population of cells to bind to the one or more specific binding substances,
(c) exposing the mixed population of cells to one or more test regents or stimuli, wherein the first cell type has a differential response to the one or more test reagents or stimuli as compared to the other cells in the mixed population of cells;
(d) detecting the differential response of the first cell type to the one or more test reagents or stimuli, wherein if the differential response is detected, then the first cell type is present in the mixture of cells.

19. The method of claim 18, wherein the differential response is a different strength of response of the first cell type to the one or more test reagents or stimuli.

20. The method of claim 18, wherein the differential response is a different cell morphology displayed by the first cell type in response to one or more test reagents or stimuli.

21. The method of claim 18, wherein the differential response is a different cell response of the first cell type to the one or more test reagents or stimuli over time.

22. The method of claim 18, wherein the differential response is a different response kinetic of the first type of cells over time.

23. The method of claim 18, wherein the percentage of the first type of cells in the mixed population of cells is determined.

24. The method of claim 18, wherein the one or more test reagents or stimuli are expressed by one or more cells of the mixed population of cells present on the biosensor surface.

25. A method of detection of responses of a first population of cells to one or more test reagents or stimuli comprising:
(a) (i) immobilizing one or more extracellular matrix ligands to a surface of a colorimetric resonant reflectance biosensor, a grating-based waveguide biosensor, or a dielectric film stack biosensor, wherein the first population of cells have cell surface receptors specific for the one or more extracellular matrix ligands; and adding the first population of cells to the biosensor; or
(ii) mixing the first population of cells with one or more extracellular matrix ligands, wherein the first population of cells have cell surface receptors specific for the one or more extracellular matrix ligands; and adding the first population of cells with one or more extracellular matrix ligands to a surface of the colorimetric resonant reflectance biosensor, the grating-based waveguide biosensor, or the dielectric film stack biosensor;
(b) adding a gel, gel-like substance; or a second population of cells to the biosensor surface;
(c) adding the one or more test reagents or stimuli to the gel or gel-like substance, or the second population of cells; and
(d) detecting responses of the first population of cells to the one or more test reagents or stimuli.

26. The method of claim 25, wherein the one or more test reagents or stimuli are a chemotactic agent or a third population of cells that produce test reagents or stimuli.

27. The method of claim 25, wherein the second population of cells is a population of epithelial cells or a population of endothelial cells.

28. The method of claim 25, wherein the first population of cells is a population of stem cells.

29. The method of claim 25, wherein no detection labels are used.

30. The method of claim 25, further comprising detecting the responses of the second population of cells.

31. The method of claim 25, wherein responses of the first population of cells or second population of cells to the one or more stimuli is detected by monitoring the peak wavelength value over one or more time periods or by monitoring the change in effective refractive index over one or more time periods.

32. The method of claim 25, wherein the responses of the first population of cells or second population of cells are detected in real time.

33. A method of detection of responses of a first population cells to a one or more test reagents or stimuli comprising:
(a) adding one or more test reagents or stimuli to a surface of a colorimetric resonant reflectance biosensor, a grating-based waveguide biosensor, or dielectric film stack biosensor;
(b) adding basement membrane matrix, alginate gel, collagen gel, agarose gel, synthetic hydrogel, or a second population of cells to the biosensor surface;
(c) mixing the first population of cells with one or more extracellular matrix ligands, wherein the first population of cells have cell surface receptors specific for the one or more extracellular matrix ligands; and adding the first population of cells to the biosensor;
(d) detecting the responses of the first population cells to the one or more test reagents or stimuli.

34. The method of claim 33, wherein the one or more test reagents or stimuli are a chemotactic agent or a third population of cells that produce test reagents or stimuli.

35. The method of claim 33, wherein the second population of cells is a population of epithelial cells or a population of endothelial cells.

36. The method of claim 33, wherein the first population of cells is a population of stem cells.

37. The method of claim 33, wherein no detection labels are used.

38. The method of claim 33, further comprising detecting the responses of the second population of cells.

39. The method of claim 33, wherein responses of the first population of cells or second population of cells to the one or more stimuli are detected by monitoring the peak wavelength value over one or more time periods or by monitoring the change in effective refractive index over one or more time periods.

40. The method of claim 33, wherein the responses of the first population of cells or second population of cells are detected in real time.

41. A method of detection of differentiation of a first population of cells comprising:
(a) adding the first population of cells to a surface of a colorimetric resonant reflectance biosensor or a dielectric film stack biosensor, wherein the biosensor has two or more surface sectors, wherein each surface sector has a grating that with a different resonance value than the other surface sectors;
(b) detecting two of more peak wavelength values from each of the two or more surface sectors; and
(c) detecting differentiation of the first population of cells on the biosensor surface.

42. The method of claim 41, wherein the differentiation is detected in real time.

43. The method of claim 41, wherein the one or more test reagents or stimuli are applied to the biosensor before the detection of two or more peak wavelength values from each of the two or more surface sectors.

44. The method of claim 41, wherein one or more peak wavelength values are detected before the one or more test reagents or stimuli are applied to the biosensor.

45. The method of claim 41, wherein the one or more test reagents or stimuli are a chemotactic agent or a third population of cells that produce test reagents or stimuli.

46. The method of claim 41, wherein the first population of cells is a population of stem cells.

47. The method of claim 41, wherein no detection labels are used.

48. A method of biological expression profiling to identify biological response signatures specific for a particular population of stem cells comprising:
(a) (i) immobilizing one or more extracellular matrix ligands to two or more surfaces of a colorimetric resonant reflectance biosensor, a grating-based waveguide biosensor, or a dielectric film stack biosensor, wherein the population of stem cells have cell surface receptors specific for the one or more extracellular matrix ligands; and adding the population of stem cells to the two or more locations of the biosensor; or
(ii) mixing the population of stem cells with one or more extracellular matrix ligands, wherein the stem cells have cell surface receptors specific for the one or more extracellular matrix ligands; and adding the population of stem cells with one or more extracellular matrix ligands to two or more surfaces of a colorimetric resonant reflectance biosensor, a grating-based waveguide biosensor or a dielectric film stack biosensor;
(b) exposing the two or more surfaces of the biosensor to two or more test reagents or stimuli;
(c) detecting responses of the stem cells to the test reagents or stimuli at each of the two or more surfaces of the biosensor;
(d) identifying the biological response signatures specific for a particular population of the stem cells to two or more test reagents or stimuli.

49. The method of claim 48, wherein detecting responses of the stem cells is done in real time.

50. A method for screening a candidate compound for its ability to modulate cell differentiation comprising: (a) adding one or more types of cells to a surface of a colorimetric resonant reflectance biosensor, a grating-based waveguide biosensor, or a dielectric film stack biosensor; (b) inducing the one or more types of cells to differentiate; (c) detecting a change in cell differentiation in the presence or absence of the candidate compound by comparing the peak wavelength values or effective changes in refractive index in the presence or absence of the candidate compound, wherein a change in cell differentiation activity in the presence of the compound relative to cell differentiation activity in the absence of the candidate compound indicates an ability of the candidate compound to modulate cell differentiation.

51. The method of claim 50, wherein the change in cell differentiation activity is an increase in cell differentiation activity, decrease in cell differentiation activity, inhibition of cell differentiation activity, increase or decrease in stem cell self-renewal, or a change in the type of differentiated cell.

52. The method of claim 50, wherein the change in cell differentiation activity is an increase or decrease in collagen production.

53. The method of claim 50, wherein the change in cell differentiation activity is an increase or decrease in mineralized nodule formation.

54. The method of claim 50, wherein the one or more types of cells are stem cells.

55. The method of claim 50, wherein the one or more types of cells are mesenchymal stem cells.

56. The method of claim 50, wherein the change in cell differention activity is detected by detecting a change in cell size, cell shape, cell membrane potential, cell metabolic activity, or cell responsiveness to signals.

57. The method of claim 50, wherein the candidate compound is an inhibitory nucleic acid molecule.

58. A method for screening a candidate compound for its ability to modulate cell differentiation comprising: (a) adding one or more types of cells to a surface of a colorimetric resonant reflectance biosensor, a grating-based waveguide biosensor, or a or a dielectric film stack biosensor; (b) inducing the one or more types of cells to differentiate; (c) detecting the production of one or more cell products of differentiation in the presence or absence of the candidate compound by comparing the peak wavelength values or effective refractive index in the presence or absence of the candidate compound, wherein a change in one or more cell products of differentiation in the presence of the candidate compound relative to one or more cell products of differentiation in the absence of the candidate compound indicates an ability of the candidate to modulate cell differentiation.

59. The method of claim 58, wherein the product of cell differentiation is collagen or mineralization nodules.

60. The method of claim 58, wherein the one or more types of cells are stem cells.

61. The method of claim 58, wherein the one or more types of cells are mesenchymal stem cells.

62. The method of claim 58, wherein the candidate compound is an inhibitory nucleic acid molecule.

63.-82. (canceled)
